SEQUENCE LISTING (201 sequences)

SEQ ID NO: 1 - protein, 123 aa, synthetic construct
EVQLQQSGPELVKPGASVKISCKASGYSFIGYFMNWVMQSHGRSLEWIGRINPYNGYTFYNQKFKGKATLTVDKSSSTAHMELRSLASEDSAVYYCARHFRYDGVFYYAMDYWGQGTSVTVSS

SEQ ID NO: 2 - protein, 115 aa, synthetic construct
QLVLTQSSSASFSLGASAKLTCTLSSQHSTFTIEWYQQQPLKPPKYVMDLKKDGSHSTGDGVPDRFSGSSSGADRYLSISNIQPEDEATYICGVGDTIKEQFVYVFGGGTKVTVL

SEQ ID NO: 3 - protein, 122 aa, synthetic construct
EVQLVESGGGLVQPGGSLRLSCAASGFAFSGYYMAWVRQAPGKGLEWVANINYPGSSTYYLDSVKGRFTISRDNAKNSLYLQMNSLRAEDTAVYYCARGDYYGTTYWYFDVWGQGTTVTVSS

SEQ ID NO: 4 - protein, 106 aa, synthetic construct
DIQMTQSPSSLSASVGDRVTITCRASQSISSYLNWYQQKPGKAPKLLIYYTSRLQSGVPSRFSGSGSGTDFTLTISSLQPEDFATYYCQQGRRLWSFGGGTKVEIK

SEQ ID NO: 5 - protein, 118 aa, synthetic construct
QVQLQQPGAELVKPGASLKLSCKSSGYTFTSYWMHWVRQRPGHGLEWIGEIDPSDSYKDYNQKFKDKATLTVDRSSNTAYMHLSSLTSDDSAVYYCARAITTTPFDFWGQGTTLTVSS

SEQ ID NO: 6 - protein, 108 aa, synthetic construct
DIVLTQSPATLSVTPGDSVSLSCRASQSISNNLHWYQQKSHESPRLLIKYASQSISGIPSRFSGSGSGTDFTLSINSVETEDFGVYFCQQSNTWPYTFGGGTKLEIKR

SEQ ID NO: 7 - protein, 119 aa, synthetic construct
EVQLQQSGAELVKPGASVKLSCTASGFNIKDTYVHWVKQRPEQGLEWIGRIDPANGYTKYDPKFQGKATITADTSSNTAYLQLSSLTSEDTAVYYCVRPLYDYYAMDYWGQGTSVTVSS

SEQ ID NO: 8 - protein, 107 aa, synthetic construct
DILMTQSPSSMSVSLGDTVSITCHASQGISSNIGWLQQKPGKSFMGLIYYGTNLVDGVPSRFSGSGSGADYSLTISSLDSEDFADYYCVQYAQLPYTFGGGTKLEIK

SEQ ID NO: 9 - protein, 120 aa, synthetic construct
EVQLVESGGGLVQPKGSLKLSCAASGFSFNTYAMNWVRQAPGKGLEWIAHIRSKSNNFATYYADSVKDRFSISRDASENILFLQMNNLKTEDTAMYYCVRQGGDFPMDYWGQGTSVTVSS

SEQ ID NO: 10 - protein, 106 aa, synthetic construct
QIVLTQSPAIMSASPGEKVTISCSASSSVSYMYWYQQKPGSSPKPWIYRTSNLASGVPARFSGSGSGTSYSLTISNMEAEDAAAYYCQQYHSYPTTFGGGTKLEVK

SEQ ID NO: 11 - protein, 115 aa, synthetic construct
QVQLQQSGPELVKPGASVKMSCKASGYAFSSSWLNWVRQRPGKGLEWIGRIYPGDGENHYNGKFKGKATLTADKSSSTGYMQLSSLTSEDSAVYFCASYYEGGYWGQGTLITVSA

SEQ ID NO: 12 - protein, 112 aa, synthetic construct
DIVMTQAAPSIPVTPGESVSISCRSDKSLLHSNGNTYLFWFLQRPGQSPQLLIYRMSNLASGVPDRFSGSGSGTAFTLRISGVEAEDVGVYYCMQHLEYPYTFGGGTKLEIK

SEQ ID NO: 13 - protein, 124 aa, synthetic construct
EVQLVESGGGLVRPGGSLRLSCAASGFTFSNYDMHWVRQATGKGLEWVSAITAAGDIYYPGSVKGRFTISRENAKNSLYLQMNSLRAGDTAVYYCARGRYSGSGSYYNDWFDPWGQGTLVTVSS

SEQ ID NO: 14 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 15 - protein, 136 aa, synthetic construct
MDSRLNLVFLVLILKGVQCDVQLVESGGGLVQPGGSRKLSCAASGFTFSSFGMHWVRQAPEKGLEWVAYISSGSSTIYYADTVKGRFTISRDNPKNTLFLQMTSLRSEDTAMYYCARVHYYYFDYWGQGTTLTVSS

SEQ ID NO: 16 - protein, 123 aa, synthetic construct
MRPSIQFLGLLLFWLHGAQCDIQMTQSPSSLSASLGGKVTITCKASQDINKYIAWYQHKPGKGPRLLIHYTSTLQPGIPSRFSGSGSGRDYSFSISNLEPEDIATYYCLQYDNLWTFGGGTKL

SEQ ID NO: 17 - protein, 134 aa, synthetic construct
MAWVWTLLFLMAAAQSIQAQIQLVQSGPELKKPGETVKISCKASGYTFTDYSMHWVKQAPGKGLKWMGWINTETGEPTYADDFKGRFAFSLETSASTAYLQINNLKNEDTATYFCARWLLFDYWGQGTTLTVSS

SEQ ID NO: 18 - protein, 130 aa, synthetic construct
MESQTQVLMFLLLWVSGACADIVMTQSPSSLAMSVGQKVTMSCKSSQSLLNSSNQKNYLAWYQQKPGQSPKLLVYFASTRESGVPDRFIGSGSGTDFTLTISSVQAEDLADYFCQQHYSTPLTFGAGTKL

SEQ ID NO: 19 - protein, 120 aa, synthetic construct
EVKLEESGGGLVQPGGSMKLSCVASGFTFSYYWMNWVRQSPEKGLEWVAEIRLKSNNYATHYAESVKGRFTISRDDSKNNVYLQMNNLRAEDTGIYYCNRRDEYYAMDYWGQGTSVSVSS

SEQ ID NO: 20 - protein, 111 aa, synthetic construct
DIVLTQSPGSLAVSLGQRATISCRASESVDNFGISFMNWFQQKPGQPPRLLIYGASNQGSGVPARFSGSGSGTDFSLNIHPVEEDDAAMYFCQQSKEVPWTFGGGTKLEIK

SEQ ID NO: 21 - protein, 120 aa, synthetic construct
QIQLVQSGPELKKPGETVKISCKASGYTFTNYGMNWVKQAPGKGLKWMGWLNTYTGESIYPDDFKGRFAFSSETSASTAYLQINNLKNEDMATYFCARGDYGYDDPLDYWGQGTSVTVSS

SEQ ID NO: 22 - protein, 112 aa, synthetic construct
DIVMTQAAPSVPVTPGESVSISCRSSKSLLHTNGNTYLHWFLQRPGQSPQLLIYRMSVLASGVPDRFSGSGSGTAFTLSISRVEAEDVGVFYCMQHLEYPLTFGAGTKLELK

SEQ ID NO: 23 - protein, 118 aa, synthetic construct
QVHLVESGGGVVQPGRSLRLSCAASGFTFSSYGMHWVRQAPGKGLEWVAVIWYDGSNYYYTDSVKGRFTISRDNSKNTLYLQMNSLRAEDTAVYYCARDLGAAASDYWGQGTLVTVSS

SEQ ID NO: 24 - protein, 107 aa, synthetic construct
AIQLTQSPSSLSASVGDRVTITCRASQGINSALAWYQQKPGKAPKLLIYDASSLESGVPSRFSGSGSGTDFTLTISSLQPEDFATYYCQQFNSYPHTFGQGTKLEIK

SEQ ID NO: 25 - protein, 146 aa, synthetic construct
MDLMCKKMKHLWFFLLLVAAPRWVLSQLQLQESGPGLVKPSETLSLTCTVSGGSIISKSSYWGWIRQPPGKGLEWIGSIYYSGSTFYNPSLKSRVTISVDTSKNQFSLKLSSVTAADTAVYYCARLTVAEFDYWGQGTLVTVSSAS

SEQ ID NO: 26 - protein, 129 aa, synthetic construct
MEAPAQLLFLLLLWLPDTTGEIVLTQSPATLSLSPGERATLSCRASQSVSSFLAWYQQKPGQAPRLLIYDASNRATGIPARFSGSGSGTDFTLTISSLEPEDFAVYYCQQRSNWPLTFGPGTKVDIKRT

SEQ ID NO: 27 - protein, 146 aa, synthetic construct
MDLMCKKMKHLWFFLLLVAAPRWVLSQLQLQESGPGLVKPSETLSLTCTVSGGSISSRSNYWGWIRQPPGKGLEWIGNVYYRGSTYYNSSLKSRVTISVDTSKNQFSLKLSSVTVADTAVYYCARLSVAEFDYWGQGILVTVSSAS

SEQ ID NO: 28 - protein, 129 aa, synthetic construct
MEAPAQLLFLLLLWLPDTTGEIVLTQSPATLSLSPGERATLSCRASQSVSSFLAWYQQKPGQAPRLLIYDASNRATGSPARFSGSGSGTDFTLTISSLEPEDFAVYYCQQRSDWPLTFGPGTKVDIKRT

SEQ ID NO: 29 - protein, 154 aa, synthetic construct
MDLMCKKMKHLWFFLLLVAAPRWVLSQLQLQESGPGLVKPSETLSLTCTVSGGSISSSSYYWGWVRQPPGKGLEWIGSIHYSGSTFYNPSLKSRVTISVDTSKNQFSLKLSSVTAADTTVYYCARQGSTVVRGVYYYGMDVWGQGTTVTVSSAS

SEQ ID NO: 30 - protein, 131 aa, synthetic construct
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLYTFGQGTKLEIKRT

SEQ ID NO: 31 - protein, 145 aa, synthetic construct
MDWTWRILFLVAAATSAHSQVQLVQSGAEMKKPGASVKVSCKTSGYTFTNYKINWVRQAPGQGLEWMGWMNPDTDSTGYPQKFQGRVTMTRNTSISTAYMELSSLRSEDTAVYYCARSYGSGSYYRDYYYGMDVWGQGTTVTVSS

SEQ ID NO: 32 - protein, 128 aa, synthetic construct
MEAPAQLLFLLLLWLPDTTGEIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKR

SEQ ID NO: 33 - protein, 139 aa, synthetic construct
MEFGLSWLFLVAILKGVQCEVQLLESGGGLVQPGRSLRLSCAASGFTFSSYAMSWVRQAPGKGLEWVSAISGSGGSRYYADSVKGRFTISRDNSKNTLYLQMNSLRAEDTAVYYCAKESSGWFGAFDYWGQGTLVTVSS

SEQ ID NO: 34 - protein, 127 aa, synthetic construct
MSPSQLIGFLLLWVPASRGEIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPITFGQGTRLEIKR

SEQ ID NO: 35 - protein, 123 aa, synthetic construct
EVILVESGGGLVKPGGSLKLSCAASGFTFSSFAMSWVRQTPEKRLEWVATISSGSIYIYYTDGVKGRFTISRDNAKNTVHLQMSSLRSEDTAMYYCARRGIYYGYDGYAMDYWGQGTSVTVSS

SEQ ID NO: 36 - protein, 112 aa, synthetic construct
AVVMTQTPLSLPVSLGDQASISCRSSQSLVHSNGNTYLHWYMQKPGQSPKVLIYKVSNRFSGVPDRFSGSGSGTDFTLKISRVEADDLGIYFCSQSTHIPLAFGAGTKLELK

SEQ ID NO: 37 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 38 - protein, 133 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 39 - protein, 518 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 40 - protein, 340 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 41 - protein, 166 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 42 - protein, 114 aa, synthetic construct
NWVNVISDLKKIEDLIQSMHIDATLYTESDVHPSCKVTAMKCFLLELQVISLASGDASIHDTVENLIILANNSLSSNGAVTESGCKECEELEEKNIKEFLQSFVHIVQMFINTS

SEQ ID NO: 43 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 44 - protein, 77 aa, Homo sapiens
MAPRRARGCRTLGLPALLLLLLLRPPATRGITCPPPMSVEHADIWVKSYSLYSRERYICNSGFKRKAGTSSLTECVL

SEQ ID NO: 45 - protein, 20 aa, synthetic construct
SGGSGGGGSGGGSGGGGSLQ

SEQ ID NO: 46 - protein, 133 aa, Homo sapiens [sequence omitted]

SEQ ID NO: 47 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 48 - protein, 133 aa, Homo sapiens [sequence omitted]

SEQ ID NO: 49 - protein, 138 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRG

SEQ ID NO: 50 - protein, 238 aa, synthetic construct
DLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPASRLLLDQVGMKENEDKYMCESSGFYPEAINITWEKQTQKFPHPIEISEDVITGPTIKNMDGTFNVTSCLKLNSSQEDPGTVYQCVVRHASLHTPLRSNFTLTAARHSLSETEKTDNFS

SEQ ID NO: 51 - protein, 284 aa, synthetic construct
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHW

SEQ ID NO: 52 - protein, 287 aa, synthetic construct
AEPHSLRYNLMVLSQDESVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKVLVLQSQRTD

SEQ ID NO: 53 - protein, 191 aa, synthetic construct
GWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPG

SEQ ID NO: 54 - protein, 329 aa, synthetic construct
QDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGG

SEQ ID NO: 55 - protein, 323 aa, synthetic construct
WPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRN

SEQ ID NO: 56 - protein, 330 aa, synthetic construct
QNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDPPTTIPPPTTTTTTTTTTTTTILTIITDSRAGEEGSIRAVDH

SEQ ID NO: 57 - protein, 155 aa, synthetic construct
QRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 58 - protein, 294 aa, synthetic construct
WTYHYSEKPMNWQRARRFCRDNYTDLVAIQNKAEIEYLEKTLPFSRSYYWIGIRKIGGIWTWVGTNKSLTEEAENWGDGEPNNKKNKEDCVEIYIKRNKDAGKWNDDACHKLKAALCYTASCQPWSCSGHGECVEIINNYTCNCDVGYYGPQCQFVIQCEPLEAPELGTMDCTHPLGNFSFSSQCAFSCSEGTNLTGIEETTCGPFGNWSSPEPTCQVIQCEPLSAPDLGIMNCSHPLASFSFTSACTFICSEGTELIGKKKTICESSGIWSNPSPICQKLDKSFSMIKEGDYN

SEQ ID NO: 59 - protein, 243 aa, synthetic construct
RYLQVSQQLQQTNRVLEVTNSSLRQQLRLKITQLGQSAEDLQGSRRELAQSQEALQVEQRAHQAAEGQLQACQADRQKTKETLQSEEQQRRALEQKLSNMENRLKPFFTCGSADTCCPSGWIMHQKSCFYISLTSKNWQESQKQCETLSSKLATFSEIYPQSHSYYFLNSLLPNGGSGNSYWTGLSSNKDWKLTDDTQRTRTYAQSSKCNKVHKTWSWWTLESESCRSSLPYICEMTAFRFPD

SEQ ID NO: 60 - protein, 124 aa, synthetic construct
KLTRDSQSLCPYDWIGFQNKCYYFSKEEGDWNSSKYNCSTQHADLTIIDNIEEMNFLRRYKCSSDHWIGLKMAKNRTGQWVDGATFTKSFGMRGSEGCAYLSDDGAATARCYTERKWICRKRIH

SEQ ID NO: 61 - protein, 194 aa, synthetic construct
QGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARS

SEQ ID NO: 62 - protein, 133 aa, synthetic construct
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 63 - protein, 149 aa, synthetic construct
MQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 64 - protein, 205 aa, synthetic construct
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 65 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 66 - protein, 455 aa, synthetic construct; identical to SEQ ID NO: 65

SEQ ID NO: 67 - protein, 413 aa, synthetic construct
FRGPLLPNRPFTTVWNANTQWCLERHGVDVDVSVFDVVANPGQTFRGPDMTIFYSSQGTYPYYTPTGEPVFGGLPQNASLIAHLARTFQDILAAIPAPDFSGLAVIDWEAWRPRWAFNWDTKDIYRQRSRALVQAQHPDWPAPQVEAVAQDQFQGAARAWMAGTLQLGRALRPRGLWGFYGFPDCYNYDFLSPNYTGQCPSGIRAQNDQLGWLWGQSRALYPSIYMPAVLEGTGKSQMYVQHRVAEAFRVAVAAGDPNLPVLPYVQIFYDTTNHFLPLDELEHSLGESAAQGAAGVVLWVSWENTRTKESCQAIKEYMDTTLGPFILNVTSGALLCSQALCSGHGRCVRRTSHPKALLLLNPASFSIQLTPGGGPLSLRGALSLEDQAQMAVEFKCRCYPGWQAPWCERKSMW

SEQ ID NO: 68 - protein, 551 aa, synthetic construct
YNFFPRKPKWDKNQITYRIIGYTPDLDPETVDDAFARAFQVWSDVTPLRFSRIHDGEADIMINFGRWEHGDGYPFDGKDGLLAHAFAPGTGVGGDSHFDDDELWTLGEGQVVRVKYGNADGEYCKFPFLFNGKEYNSCTDTGRSDGFLWCSTTYNFEKDGKYGFCPHEALFTMGGNAEGQPCKFPFRFQGTSYDSCTTEGRTDGYRWCGTTEDYDRDKKYGFCPETAMSTVGGNSEGAPCVFPFTFLGNKYESCTSAGRSDGKMWCATTANYDDDRKWGFCPDQGYSLFLVAAHEFGHAMGLEHSQDPGALMAPIYTYTKNFRLSQDDIKGIQELYGASPDIDLGTGPTPTLGPVTPEICKQDIVFDGIAQIRGEIFFFKDRFIWRTVTPRDKPMGPLLVATFWPELPEKIDAVYEAPQEEKAVFFAGNEYWIYSASTLERGYPKPLTSLGLPPDVQRVDAAFNWSKNKKTYIFAGDKFWRYNEVKKKMDPGFPKLIADAWNAIPDNLDAVVDLQGGGHSYFFKGAYYLKLENQSLKSVKFGSIKSDWLGC

SEQ ID NO: 69 - protein, 5 aa, synthetic construct
GGGGS

SEQ ID NO: 70 - protein, 10 aa, synthetic construct
GGGGSGGGGS

SEQ ID NO: 71 - protein, 15 aa, synthetic construct
GGGGSGGGGSGGGGS

SEQ ID NO: 72 - protein, 17 aa, synthetic construct
DVPSGPGGGGGSGGGGS

SEQ ID NO: 73 - protein, 14 aa, synthetic construct
AEAAAKEAAAKAAA

SEQ ID NO: 74 - protein, 19 aa, synthetic construct
AEAAAKEAAAKEAAAKAAA

SEQ ID NO: 75 - protein, 24 aa, synthetic construct
AEAAAKEAAAKEAAAKEAAAKAAA

SEQ ID NO: 76 - protein, 29 aa, synthetic construct
AEAAAKEAAAKEAAAKEAAAKEAAAKAAA

SEQ ID NO: 77 - protein, 122 aa, Mus sp.
EIQLQQSGPELMKPGASLKISCKTSGYSFTSYYMHWVKQSHGQSLEWIGFIDPFKVITGYNHNFRGKATLTVDRSSTTAYMHLRSLTSEDSAVYYCARRYYSDYDGYALDYWGQGTSVTVSS

SEQ ID NO: 78 - protein, 113 aa, Mus sp.
DVVMTQTPLSLPVSLGDQASIFCRSSQSLVHSDGNTYLHWYLQKPGQSPKLLIYKVSNRFSGVPDRFSGSGSGTDFTLKISRVEAEDLGVYFCSQSTHVPPYTFGGGTKLEIK

SEQ ID NO: 79 - protein, 122 aa, synthetic construct
QIQLQESGPGLVKPSETLSLTCTTSGYSFTSYYMHWIRQPPGKGLEWIGFIDPFKVITGYNHNFRGRVTISVDRSKTQASLKLSSVTAADTAVYYCARRYYSDYDGYALDYWGQGTLVTVSS

SEQ ID NO: 80 - protein, 122 aa, synthetic construct
EIQLVQSGAEVKKPGATVKISCKTSGYSFTSYYMHWVQQAPGKGLEWMGFIDPFKVITGYNHNFRGRVTITVDRSTTTAYMELSSLRSEDTAVYYCARRYYSDYDGYALDYWGQGTLVTVSS

SEQ ID NO: 81 - protein, 122 aa, synthetic construct
QIQLVQSGAEVKKTGSSVKVSCKTSGYSFTSYYMHWVRQAPGQALEWMGFIDPFKVITGYNHNFRGRVTITVDRSMTTAYMELSSLRSEDTAMYYCARRYYSDYDGYALDYWGQGTLVTVSS

SEQ ID NO: 82 - protein, 122 aa, synthetic construct
QIQLVQSGAEVKKPGASVKVSCKTSGYSFTSYYMHWVRQAPGQGLEWMGFIDPFKVITGYNHNFRGRVTSTVDRSITTAYMELSRLRSDDTVVYYCARRYYSDYDGYALDYWGQGTLVTVSS

SEQ ID NO: 83 - protein, 113 aa, synthetic construct
EVVMTQSPGTLSLSPGERATLSCRSSQSLVHSDGNTYLHWYQQKPGQAPRLLIYKVSNRFSGIPDRFSGSGSGTDFTLTISRLEPEDFAVYFCSQSTHVPPYTFGGGTKVEIK

SEQ ID NO: 84 - protein, 113 aa, synthetic construct
EVVMTQSPATLSLSPGERATLSCRSSQSLVHSDGNTYLHWYQQKPGQAPRLLIYKVSNRFSGIPARFSGSGSGTDFTLTISSLEPEDFAVYFCSQSTHVPPYTFGGGTKVEIK

SEQ ID NO: 85 - protein, 113 aa, synthetic construct
EVVMTQSPATLSVSPGERATLSCRSSQSLVHSDGNTYLHWYQQKPGQAPRLLIYKVSNRFSGIPARFSGSGSGTEFTLTISSLQSEDFAVYFCSQSTHVPPYTFGGGTKVEIK

SEQ ID NO: 86 - protein, 113 aa, synthetic construct
VVWMTQSPSLLSASTGDRVTISCRSSQSLVHSDGNTYLHWYQQKPGKAPELLIYKVSNRFSGVPSRFSGSGSGTDFTLTISCLQSEDFATYFCSQSTHVPPYTFGGGTKVEIK

SEQ ID NO: 87 - protein, 7 aa, synthetic construct
FTSYYMH

SEQ ID NO: 88 - protein, 17 aa, synthetic construct
FIDPFKVITGYNHNFRG

SEQ ID NO: 89 - protein, 13 aa, synthetic construct
RYYSDYDGYALDY

SEQ ID NO: 90 - protein, 16 aa, synthetic construct
RSSQSLVHSDGNTYLH

SEQ ID NO: 91 - protein, 7 aa, synthetic construct
KVSNRFS

SEQ ID NO: 92 - protein, 10 aa, synthetic construct
SQSTHVPPYT

SEQ ID NO: 93 - protein, 414 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 94 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 95 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 96 - protein, 507 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTGPFSVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 97 - protein, 426 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 98 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 99 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 100 - protein, 137 aa, Homo sapiens
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 101 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 102 - protein, 162 aa, Homo sapiens
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 103 - protein, 101 aa, Homo sapiens
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIF

SEQ ID NO: 104 - protein, 93 aa, Homo sapiens
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVEL

SEQ ID NO: 105 - protein, 79 aa, Homo sapiens
ALQCFCHLCTKDNFTCVTDGLCFVSVTETTDKVIHNSMCIAEIDLIPRDRPFVCAPSSKTGSVTTTYCCNQDHCNKIEL

SEQ ID NO: 106 - protein, 851 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 107 - protein, 850 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 108 - protein, 767 aa, Homo sapiens
GPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTV

SEQ ID NO: 109-116 - skipped (8 entries)

SEQ ID NO: 117 - protein, 361 aa, Homo sapiens
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 118 - protein, 394 aa, Homo sapiens
LSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 119 - protein, 389 aa, Homo sapiens
LSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 120 - protein, 470 aa, Homo sapiens
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 121 - protein, 474 aa, Homo sapiens
LQCFCHLCTKDNFTCVTDGLCFVSVTETTDKVIHNSMCIAEIDLIPRDRPFVCAPSSKTGSVTTTYCCNQDHCNKIELPTTGPFSVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 122 - protein, 393 aa, Homo sapiens
LQCFCHLCTKDNFTCVTDGLCFVSVTETTDKVIHNSMCIAEIDLIPRDRPFVCAPSSKTGSVTTTYCCNQDHCNKIELPTTGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 123 - protein, 545 aa, Homo sapiens
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 124 - protein, 570 aa, Homo sapiens
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 125 - protein, 831 aa, Homo sapiens
GPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 126 - protein, 830 aa, Homo sapiens
GPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 127-191 - skipped (65 entries)

SEQ ID NO: 192 - protein, 481 aa, synthetic construct
  VARIANT 330: note = May be absent
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 193 - protein, 481 aa, synthetic construct
  VARIANT 330: note = May be absent
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 194 - protein, 378 aa, synthetic construct
  VARIANT 227: note = May be absent
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 195 - protein, 378 aa, synthetic construct
  VARIANT 227: note = May be absent
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 196 - protein, 481 aa, synthetic construct
  VARIANT 481: note = May be absent
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 197 - protein, 481 aa, synthetic construct
  VARIANT 481: note = May be absent
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 257 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 199 - protein, 258 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 201 - protein, 27 aa, synthetic construct
  VARIANT 12..26: note = EAAAK repeats may be deleted
AEAAAKEAAAKEAAAKEAAAKEAAAKA

(19) United States
(12) Patent Application Publication
(10) Pub. No.: US 2025/0074998 A1
(43) Pub. Date: Mar. 6, 2025

(54) MULTIFUNCTIONAL MOLECULES AND USES THEREOF

(71) Applicant: Marengo Therapeutics, Inc., Cambridge, MA (US)

(72) Inventors: Andreas Loew, Boston, MA (US); Ilaria Lamberto, Arlington, MA (US); John Leonard Herrmann, Winchester, MA (US); Brian Edward Vash, Cambridge, MA (US)

(21) Appl. No.: 18/659,544

(22) Filed: May 9, 2024

Related U.S. Application Data
(63) Continuation of application No. 16/980,771, filed on Sep. 14, 2020, now abandoned.
(60) Provisional application No. 62/642,689, filed on Mar. 14, 2018.

(51) Int. Cl. C07K 16/30; A61K 39/395; A61K 45/06; C07K 14/54; C07K 14/57; C07K 14/71; C07K 16/28; C12N 9/26; C12N 9/50

(52) U.S. Cl. CPC C07K 16/30; A61K 39/39558; A61K 45/06; C07K 14/5443; C07K 14/57; C07K 14/71; C07K 16/2809; C12N 9/2474; C12N 9/50; C07K 2317/31; C07K 2319/30

(57) ABSTRACT

Multispecific molecules that include a first tumor-targeting moiety; a second tumor-targeting moiety; and one, two or all of: an immune cell engager (e.g., chosen from an NK cell engager, a T cell engager, a B cell engager, a dendritic cell engager, or a macrophage cell engager); a cytokine molecule or a modulator of a cytokine molecule; and/or a stromal modifying moiety are disclosed. Additionally disclosed are nucleic acids encoding the same, methods of producing the aforesaid molecules, and methods of treating a cancer using the aforesaid molecules.

### RELATED APPLICATION

This application is a continuation of U.S. Ser. No. 16/980,771 filed Sep. 14, 2020, which is a U.S. National Phase Application under 35 U.S.C. § 371 of International Application No. PCT/US2019/022284 filed Mar. 14, 2019, which claims priority to U.S. Ser. No. 62/642,689 filed Mar. 14, 2018, the contents of which are incorporated herein by reference in their entireties.

### SEQUENCE LISTING

The instant application contains a Sequence Listing which has been submitted electronically in XML format and is hereby incorporated by reference in its entirety. Said XML copy, created on May 9, 2024, is named 53676-727.301_SL.xml and is 156,568 bytes in size.

### BACKGROUND

Myeloproliferative neoplasms (MPNs) are a group of conditions that cause blood cells to grow abnormally in the bone marrow. Common myeloproliferative neoplasms include primary or idiopathic myelofibrosis (MF), essential thrombocytosis (ET), polycythemia vera (PV), and chronic myelogenous leukemia (CML). Primary myelofibrosis is a chronic blood cancer in which excessive scar tissue forms in the bone marrow and impairs its ability to produce normal blood cells. Given the ongoing need for improved treatment of myeloproliferative neoplasms such as myelofibrosis, new compositions and treatments targeting myeloproliferative neoplasms are highly desirable.

### SUMMARY OF THE INVENTION

The disclosure relates, inter alia, to novel multispecific or multifunctional molecules that include (i) a first tumor-targeting moiety that binds to a first tumor antigen; and (ii) a second tumor-targeting moiety that binds to a second tumor antigen, wherein the first and second tumor antigens are each independently chosen from: CD34, CD41, G6B, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1. In some embodiments, the multifunctional molecule further comprises a third tumor-targeting moiety that binds to a third tumor antigen, wherein the third tumor antigen is chosen from: CD34, CD41, G6B, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1. In some embodiments, the multifunctional molecule further comprises one, two, or all of: (iii) an immune cell engager chosen from a T cell engager, an NK cell engager, a B cell engager, a dendritic cell engager, or a macrophage cell engager; (iv) a cytokine molecule or a modulator of a cytokine molecule; and (v) a stromal modifying moiety. The terms “multispecific” or “multifunctional” are used interchangeably herein.

Without wishing to be bound by theory, the multispecific or multifunctional molecules disclosed herein are expected to target (e.g., localize, bridge and/or activate) an immune cell (e.g., an immune effector cell chosen form an NK cell, a T cell, a B cell, a dendritic cell or a macrophage), at a target cell, e.g., a cancer cell, expressing the first, second, and/or third tumor antigens, and/or alter the tumor stroma, e.g., alter the tumor microenvironment near the cancer site. Increasing the proximity and/or activity of the immune cell using the multispecific molecules described herein is expected to enhance an immune response against the target cell (e.g., the cancer cell), thereby providing a more effective therapy (e.g., a more effective cancer therapy). Without being bound by theory, a targeted, localized immune response against the target cell (e.g., the cancer cell) is believed to reduce the effects of systemic toxicity of the multispecific molecules described herein.

Accordingly, provided herein are, inter alia, multispecific molecules (e.g., multispecific or multifunctional antibody molecules) that include the aforesaid moieties, nucleic acids encoding the same, methods of producing the aforesaid molecules, and methods of treating a cancer using the aforesaid molecules.

Accordingly, in one aspect, the disclosure features a multifunctional molecule (e.g., polypeptide or nucleic acid encoding the same) that includes:
(i) a first tumor-targeting moiety that binds to a first tumor antigen, and
(ii) a second tumor-targeting moiety that binds to a second tumor antigen, wherein:
the first and second tumor antigens are each independently chosen from: CD34, CD41, G6B, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1.

In some embodiments, the first tumor antigen is different from the second tumor antigen.

In another aspect, the disclosure features a multifunctional molecule (e.g., polypeptide or nucleic acid encoding the same) that includes:
(i) a first tumor-targeting moiety that binds to a first tumor antigen;
(ii) a second tumor-targeting moiety that binds to a second tumor antigen; and one, two, or all of
(iii) an immune cell engager chosen from a T cell engager, an NK cell engager, a B cell engager, a dendritic cell engager, or a macrophage cell engager;
(iv) a cytokine molecule or a modulator of a cytokine molecule; and
(v) a stromal modifying moiety, wherein:
the first and second tumor antigens are each independently chosen from: CD34, CD41, G6B, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1. In some embodiments, the first tumor antigen is different from the second tumor antigen.

In some embodiments, the multifunctional molecule further comprises (vi) a third tumor-targeting moiety that binds to a third tumor antigen. In some embodiments, the third tumor antigen is chosen from: CD34, CD41, G6B, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1. In some embodiments, the third tumor antigen is different from the first or second tumor antigen.

In some embodiments, the first and second tumor antigens are present on the same tumor cell. In some embodiments, the first and third tumor antigens are present on the same tumor cell. In some embodiments, the second and third tumor antigens are present on the same tumor cell. In some embodiments, the first, second, and third tumor antigens are present on the same tumor cell.

In some embodiments, the first and second tumor antigens are present on different tumor cells. In some embodiments, the first and third tumor antigens are present on different tumor cells. In some embodiments, the second and third tumor antigens are present on different tumor cells. In some embodiments, the first, second, and third tumor antigens are present on different tumor cells.

In some embodiments, the first, second, and/or third tumor antigens show higher expression in a tumor cell, e.g., a myeloproliferative neoplasm cell, than a non-tumor cell. In some embodiments, the expression of the first, second, and/or third tumor antigens in a tumor cell, e.g., a myeloproliferative neoplasm cell, is at least 1.5, 2, 4, 6, 8, or 10-fold higher than the expression of the first, second, and/or third tumor antigens in a non-tumor cell. In some embodiments, the multifunctional molecule preferentially binds to a tumor cell, e.g., a myeloproliferative neoplasm cell, over a non-tumor cell. In some embodiments, the binding between the multifunctional molecule and the tumor cell, e.g., a myeloproliferative neoplasm cell, is more than 10, 20, 30, 40, 50-fold greater than the binding between the multifunctional molecule and a non-tumor cell.

In some embodiments, the affinity, e.g., the combined affinity, of the first and second tumor-targeting moieties for a tumor cell, e.g., a myeloproliferative neoplasm cell, is greater than the affinity of a similar multifunctional molecule having only one of the first tumor-targeting moiety or the second tumor-targeting moiety. In some embodiments, the affinity, e.g., the combined affinity, of the first and second tumor-targeting moieties for a tumor cell, e.g., a myeloproliferative neoplasm cell, is at least 2, 5, 10, 20, 30, 40, 50, 75 or 100 times greater than the affinity of a similar multifunctional molecule having only one of the first tumor-targeting moiety or the second tumor-targeting moiety.

In some embodiments, the affinity, e.g., the combined affinity, of the first, second, and third tumor-targeting moieties for a tumor cell, e.g., a myeloproliferative neoplasm cell, is greater than the affinity of a similar multifunctional molecule having only one of the first tumor-targeting moiety, the second tumor-targeting moiety, or the third tumor-targeting moiety, or a similar multifunctional molecule having only two of the first tumor-targeting moiety, the second tumor-targeting moiety, or the third tumor-targeting moiety. In some embodiments, the affinity, e.g., the combined affinity, of the first, second, and third tumor-targeting moieties for a tumor cell, e.g., a myeloproliferative neoplasm cell, is at least 2, 5, 10, 20, 30, 40, 50, 75 or 100 times greater than the affinity of a similar multifunctional molecule having only one of the first tumor-targeting moiety, the second tumor-targeting moiety, or the third tumor-targeting moiety, or a similar multifunctional molecule having only two of the first tumor-targeting moiety, the second tumor-targeting moiety, or the third tumor-targeting moiety.

In some embodiments, the myeloproliferative neoplasm cell is chosen from a myelofibrosis cell, an essential thrombocythemia cell, a polycythemia vera cell, or a chronic myeloid cancer cell. In some embodiments, the myeloproliferative neoplasm cell is a myelofibrosis cell. In some embodiments, the myeloproliferative neoplasm cell is an essential thrombocythemia cell. In some embodiments, the myeloproliferative neoplasm cell is a polycythemia vera cell. In some embodiments, the myeloproliferative neoplasm cell is a chronic myeloid cancer cell. In some embodiments, the myeloproliferative neoplasm cell comprises a JAK2 mutation (e.g., a JAK2 V617F mutation). In some embodiments, the myeloproliferative neoplasm cell comprises a calreticulin mutation. In some embodiments, the myeloproliferative neoplasm cell comprises a MPL mutation.

In some embodiments, the affinity, e.g., the combined affinity, for the first and second tumor antigens of the first tumor-targeting moiety and the second tumor-targeting moiety is equal to or greater than the affinity of (iii), (iv) or (v), either alone or as part of the multifunctional molecule, for its corresponding binding member. In some embodiments, the affinity, e.g., the combined affinity, for the first and second tumor antigens of the first tumor-targeting moiety and the second tumor-targeting moiety is at least 2, 5, 10, 20, 30, 40, 50, 75 or 100 times greater than the affinity of (iii), (iv) or (v), either alone or as part of the multifunctional molecule, for its corresponding binding member.

In some embodiments, the affinity, e.g., the combined affinity, for the first, second, and third tumor antigens of the first tumor-targeting moiety, the second tumor-targeting moiety, and the third tumor-targeting moiety is equal to or greater than the affinity of (iii), (iv) or (v), either alone or as part of the multifunctional molecule, for its corresponding binding member. In some embodiments, the affinity, e.g., the combined affinity, for the first, second, and third tumor antigens of the first tumor-targeting moiety, the second tumor-targeting moiety, and the third tumor-targeting moiety is at least 2, 5, 10, 20, 30, 40, 50, 75 or 100 times greater than the affinity of (iii), (iv) or (v), either alone or as part of the multifunctional molecule, for its corresponding binding member.

In some embodiments of the aforementioned aspects, the first tumor antigen is CD34 and the second tumor antigen is CD41. In some embodiments, the first tumor antigen is CD34 and the second tumor antigen is G6B. In some embodiments, the first tumor antigen is CD41 and the second tumor antigen is G6B. In some embodiments, the first tumor antigen is CD34, the second tumor antigen is CD41, and the third tumor antigen is G6B.

In some embodiments of the aforementioned aspects, the first tumor antigen is P-selectin and the second tumor antigen is Clec2. In some embodiments, the first tumor antigen is CD34 and the second tumor antigen is P-selectin. In some embodiments, the first tumor antigen is CD41 and the second tumor antigen is P-selectin. In some embodiments, the first tumor antigen is G6B and the second tumor antigen is P-selectin. In some embodiments, the first tumor antigen is CD34 and the second tumor antigen is Clec2. In some embodiments, the first tumor antigen is CD41 and the second tumor antigen is Clec2. In some embodiments, the first tumor antigen is G6B and the second tumor antigen is Clec2. In some embodiments, the first tumor antigen is CD34, the second tumor antigen is CD41, and the third tumor antigen is P-selectin. In some embodiments, the first tumor antigen is CD34, the second tumor antigen is G6B, and the third tumor antigen is P-selectin. In some embodiments, the first tumor antigen is CD41, the second tumor antigen is G6B, and the third tumor antigen is P-selectin. In some embodiments, the first tumor antigen is CD34, the second tumor antigen is CD41, and the third tumor antigen is Clec2. In some embodiments, the first tumor antigen is CD34, the second tumor antigen is G6B, and the third tumor antigen is Clec2. In some embodiments, the first tumor antigen is CD41, the second tumor antigen is G6B, and the third tumor antigen is Clec2. In some embodiments, the first tumor antigen is CD34, the second tumor antigen is P-selectin, and the third tumor antigen is Clec2. In some embodiments, the first tumor antigen is CD41, the second tumor antigen is P-selectin, and the third tumor antigen is Clec2. In some embodiments, the first tumor antigen is G6B, the second tumor antigen is P-selectin, and the third tumor antigen is Clec2.

In some embodiments of the aforementioned aspects, the first tumor antigen is CD34 and the second tumor antigen is CD34. In some embodiments, the first tumor antigen is CD34 and the second tumor antigen is CD41. In some embodiments, the first tumor antigen is CD34 and the second tumor antigen is G6B. In some embodiments, the first tumor antigen is CD34 and the second tumor antigen is P-selectin. In some embodiments, the first tumor antigen is CD34 and the second tumor antigen is Clec2. In some embodiments, the first tumor antigen is CD34 and the second tumor antigen is cKIT. In some embodiments, the first tumor antigen is CD34 and the second tumor antigen is FLT3. In some embodiments, the first tumor antigen is CD34 and the second tumor antigen is MPL. In some embodiments, the first tumor antigen is CD34 and the second tumor antigen is ITGB3. In some embodiments, the first tumor antigen is CD34 and the second tumor antigen is ITGB2. In some embodiments, the first tumor antigen is CD34 and the second tumor antigen is GP5. In some embodiments, the first tumor antigen is CD34 and the second tumor antigen is GP6. In some embodiments, the first tumor antigen is CD34 and the second tumor antigen is GP9. In some embodiments, the first tumor antigen is CD34 and the second tumor antigen is GP1BA. In some embodiments, the first tumor antigen is CD34 and the second tumor antigen is DSC2. In some embodiments, the first tumor antigen is CD34 and the second tumor antigen is FCGR2A. In some embodiments, the first tumor antigen is CD34 and the second tumor antigen is TNFRSF10A. In some embodiments, the first tumor antigen is CD34 and the second tumor antigen is TNFRSF10B. In some embodiments, the first tumor antigen is CD34 and the second tumor antigen is TM4SF1.

In some embodiments of the aforementioned aspects, the first tumor antigen is CD41 and the second tumor antigen is CD34. In some embodiments, the first tumor antigen is CD41 and the second tumor antigen is CD41. In some embodiments, the first tumor antigen is CD41 and the second tumor antigen is G6B. In some embodiments, the first tumor antigen is CD41 and the second tumor antigen is P-selectin. In some embodiments, the first tumor antigen is CD41 and the second tumor antigen is Clec2. In some embodiments, the first tumor antigen is CD41 and the second tumor antigen is cKIT. In some embodiments, the first tumor antigen is CD41 and the second tumor antigen is FLT3. In some embodiments, the first tumor antigen is CD41 and the second tumor antigen is MPL. In some embodiments, the first tumor antigen is CD41 and the second tumor antigen is ITGB3. In some embodiments, the first tumor antigen is CD41 and the second tumor antigen is ITGB2. In some embodiments, the first tumor antigen is CD41 and the second tumor antigen is GP5. In some embodiments, the first tumor antigen is CD41 and the second tumor antigen is GP6. In some embodiments, the first tumor antigen is CD41 and the second tumor antigen is GP9. In some embodiments, the first tumor antigen is CD41 and the second tumor antigen is GP1BA. In some embodiments, the first tumor antigen is CD41 and the second tumor antigen is DSC2. In some embodiments, the first tumor antigen is CD41 and the second tumor antigen is FCGR2A. In some embodiments, the first tumor antigen is CD41 and the second tumor antigen is TNFRSF10A. In some embodiments, the first tumor antigen is CD41 and the second tumor antigen is TNFRSF10B. In some embodiments, the first tumor antigen is CD41 and the second tumor antigen is TM4SF1.

In some embodiments of the aforementioned aspects, the first tumor antigen is G6B and the second tumor antigen is CD34. In some embodiments, the first tumor antigen is G6B and the second tumor antigen is CD41. In some embodiments, the first tumor antigen is G6B and the second tumor antigen is G6B. In some embodiments, the first tumor antigen is G6B and the second tumor antigen is P-selectin. In some embodiments, the first tumor antigen is G6B and the second tumor antigen is Clec2. In some embodiments, the first tumor antigen is G6B and the second tumor antigen is cKIT. In some embodiments, the first tumor antigen is G6B and the second tumor antigen is FLT3. In some embodiments, the first tumor antigen is G6B and the second tumor antigen is MPL. In some embodiments, the first tumor antigen is G6B and the second tumor antigen is ITGB3. In some embodiments, the first tumor antigen is G6B and the second tumor antigen is ITGB2. In some embodiments, the first tumor antigen is G6B and the second tumor antigen is GP5. In some embodiments, the first tumor antigen is G6B and the second tumor antigen is GP6. In some embodiments, the first tumor antigen is G6B and the second tumor antigen is GP9. In some embodiments, the first tumor antigen is G6B and the second tumor antigen is GP1BA. In some embodiments, the first tumor antigen is G6B and the second tumor antigen is DSC2. In some embodiments, the first tumor antigen is G6B and the second tumor antigen is FCGR2A. In some embodiments, the first tumor antigen is G6B and the second tumor antigen is TNFRSF10A. In some embodiments, the first tumor antigen is G6B and the second tumor antigen is TNFRSF10B. In some embodiments, the first tumor antigen is G6B and the second tumor antigen is TM4SF1.

In some embodiments of the aforementioned aspects, the first tumor antigen is P-selectin and the second tumor antigen is CD34. In some embodiments, the first tumor antigen is P-selectin and the second tumor antigen is CD41. In some embodiments, the first tumor antigen is P-selectin and the second tumor antigen is G6B. In some embodiments, the first tumor antigen is P-selectin and the second tumor antigen is P-selectin. In some embodiments, the first tumor antigen is P-selectin and the second tumor antigen is Clec2. In some embodiments, the first tumor antigen is P-selectin and the second tumor antigen is cKIT. In some embodiments, the first tumor antigen is P-selectin and the second tumor antigen is FLT3. In some embodiments, the first tumor antigen is P-selectin and the second tumor antigen is MPL. In some embodiments, the first tumor antigen is P-selectin and the second tumor antigen is ITGB3. In some embodiments, the first tumor antigen is P-selectin and the second tumor antigen is ITGB2. In some embodiments, the first tumor antigen is P-selectin and the second tumor antigen is GP5. In some embodiments, the first tumor antigen is P-selectin and the second tumor antigen is GP6. In some embodiments, the first tumor antigen is P-selectin and the second tumor antigen is GP9. In some embodiments, the first tumor antigen is P-selectin and the second tumor antigen is GP1BA. In some embodiments, the first tumor antigen is P-selectin and the second tumor antigen is DSC2. In some embodiments, the first tumor antigen is P-selectin and the second tumor antigen is FCGR2A. In some embodiments, the first tumor antigen is P-selectin and the second tumor antigen is TNFRSF10A. In some embodiments, the first tumor antigen is P-selectin and the second tumor antigen is TNFRSF10B. In some embodiments, the first tumor antigen is P-selectin and the second tumor antigen is TM4SF1.

In some embodiments of the aforementioned aspects, the first tumor antigen is Clec2 and the second tumor antigen is CD34. In some embodiments, the first tumor antigen is Clec2 and the second tumor antigen is CD41. In some embodiments, the first tumor antigen is Clec2 and the second tumor antigen is G6B. In some embodiments, the first tumor antigen is Clec2 and the second tumor antigen is P-selectin. In some embodiments, the first tumor antigen is Clec2 and the second tumor antigen is Clec2. In some embodiments, the first tumor antigen is Clec2 and the second tumor antigen is cKIT. In some embodiments, the first tumor antigen is Clec2 and the second tumor antigen is FLT3. In some embodiments, the first tumor antigen is Clec2 and the second tumor antigen is MPL. In some embodiments, the first tumor antigen is Clec2 and the second tumor antigen is ITGB3. In some embodiments, the first tumor antigen is Clec2 and the second tumor antigen is ITGB2. In some embodiments, the first tumor antigen is Clec2 and the second tumor antigen is GP5. In some embodiments, the first tumor antigen is Clec2 and the second tumor antigen is GP6. In some embodiments, the first tumor antigen is Clec2 and the second tumor antigen is GP9. In some embodiments, the first tumor antigen is Clec2 and the second tumor antigen is GP1BA. In some embodiments, the first tumor antigen is Clec2 and the second tumor antigen is DSC2. In some embodiments, the first tumor antigen is Clec2 and the second tumor antigen is FCGR2A. In some embodiments, the first tumor antigen is Clec2 and the second tumor antigen is TNFRSF10A. In some embodiments, the first tumor antigen is Clec2 and the second tumor antigen is TNFRSF10B. In some embodiments, the first tumor antigen is Clec2 and the second tumor antigen is TM4SF1.

In some embodiments of the aforementioned aspects, the first tumor antigen is cKIT and the second tumor antigen is CD34. In some embodiments, the first tumor antigen is cKIT and the second tumor antigen is CD41. In some embodiments, the first tumor antigen is cKIT and the second tumor antigen is G6B. In some embodiments, the first tumor antigen is cKIT and the second tumor antigen is P-selectin. In some embodiments, the first tumor antigen is cKIT and the second tumor antigen is Clec2. In some embodiments, the first tumor antigen is cKIT and the second tumor antigen is cKIT. In some embodiments, the first tumor antigen is cKIT and the second tumor antigen is FLT3. In some embodiments, the first tumor antigen is cKIT and the second tumor antigen is MPL. In some embodiments, the first tumor antigen is cKIT and the second tumor antigen is ITGB3. In some embodiments, the first tumor antigen is cKIT and the second tumor antigen is ITGB2. In some embodiments, the first tumor antigen is cKIT and the second tumor antigen is GP5. In some embodiments, the first tumor antigen is cKIT and the second tumor antigen is GP6. In some embodiments, the first tumor antigen is cKIT and the second tumor antigen is GP9. In some embodiments, the first tumor antigen is cKIT and the second tumor antigen is GP1BA. In some embodiments, the first tumor antigen is cKIT and the second tumor antigen is DSC2. In some embodiments, the first tumor antigen is cKIT and the second tumor antigen is FCGR2A. In some embodiments, the first tumor antigen is cKIT and the second tumor antigen is TNFRSF10A. In some embodiments, the first tumor antigen is cKIT and the second tumor antigen is TNFRSF10B. In some embodiments, the first tumor antigen is cKIT and the second tumor antigen is TM4SF1.

In some embodiments of the aforementioned aspects, the first tumor antigen is FLT3 and the second tumor antigen is CD34. In some embodiments, the first tumor antigen is FLT3 and the second tumor antigen is CD41. In some embodiments, the first tumor antigen is FLT3 and the second tumor antigen is G6B. In some embodiments, the first tumor antigen is FLT3 and the second tumor antigen is P-selectin. In some embodiments, the first tumor antigen is FLT3 and the second tumor antigen is Clec2. In some embodiments, the first tumor antigen is FLT3 and the second tumor antigen is cKIT. In some embodiments, the first tumor antigen is FLT3 and the second tumor antigen is FLT3. In some embodiments, the first tumor antigen is FLT3 and the second tumor antigen is MPL. In some embodiments, the first tumor antigen is FLT3 and the second tumor antigen is ITGB3. In some embodiments, the first tumor antigen is FLT3 and the second tumor antigen is ITGB2. In some embodiments, the first tumor antigen is FLT3 and the second tumor antigen is GP5. In some embodiments, the first tumor antigen is FLT3 and the second tumor antigen is GP6. In some embodiments, the first tumor antigen is FLT3 and the second tumor antigen is GP9. In some embodiments, the first tumor antigen is FLT3 and the second tumor antigen is GP1BA. In some embodiments, the first tumor antigen is FLT3 and the second tumor antigen is DSC2. In some embodiments, the first tumor antigen is FLT3 and the second tumor antigen is FCGR2A. In some embodiments, the first tumor antigen is FLT3 and the second tumor antigen is TNFRSF10A. In some embodiments, the first tumor antigen is FLT3 and the second tumor antigen is TNFRSF10B. In some embodiments, the first tumor antigen is FLT3 and the second tumor antigen is TM4SF1.

In some embodiments of the aforementioned aspects, the first tumor antigen is MPL and the second tumor antigen is CD34. In some embodiments, the first tumor antigen is MPL and the second tumor antigen is CD41. In some embodiments, the first tumor antigen is MPL and the second tumor antigen is G6B. In some embodiments, the first tumor antigen is MPL and the second tumor antigen is P-selectin. In some embodiments, the first tumor antigen is MPL and the second tumor antigen is Clec2. In some embodiments, the first tumor antigen is MPL and the second tumor antigen is cKIT. In some embodiments, the first tumor antigen is MPL and the second tumor antigen is FLT3. In some embodiments, the first tumor antigen is MPL and the second tumor antigen is MPL. In some embodiments, the first tumor antigen is MPL and the second tumor antigen is ITGB3. In some embodiments, the first tumor antigen is MPL and the second tumor antigen is ITGB2. In some embodiments, the first tumor antigen is MPL and the second tumor antigen is GP5. In some embodiments, the first tumor antigen is MPL and the second tumor antigen is GP6. In some embodiments, the first tumor antigen is MPL and the second tumor antigen is GP9. In some embodiments, the first tumor antigen is MPL and the second tumor antigen is GP1BA. In some embodiments, the first tumor antigen is MPL and the second tumor antigen is DSC2. In some embodiments, the first tumor antigen is MPL and the second tumor antigen is FCGR2A. In some embodiments, the first tumor antigen is MPL and the second tumor antigen is TNFRSF10A. In some embodiments, the first tumor antigen is MPL and the second tumor antigen is TNFRSF10B. In some embodiments, the first tumor antigen is MPL and the second tumor antigen is TM4SF1.

In some embodiments of the aforementioned aspects, the first tumor antigen is ITGB3 and the second tumor antigen is CD34. In some embodiments, the first tumor antigen is ITGB3 and the second tumor antigen is CD41. In some embodiments, the first tumor antigen is ITGB3 and the second tumor antigen is G6B. In some embodiments, the first tumor antigen is ITGB3 and the second tumor antigen is P-selectin. In some embodiments, the first tumor antigen is ITGB3 and the second tumor antigen is Clec2. In some embodiments, the first tumor antigen is ITGB3 and the second tumor antigen is cKIT. In some embodiments, the first tumor antigen is ITGB3 and the second tumor antigen is FLT3. In some embodiments, the first tumor antigen is ITGB3 and the second tumor antigen is MPL. In some embodiments, the first tumor antigen is ITGB3 and the second tumor antigen is ITGB3. In some embodiments, the first tumor antigen is ITGB3 and the second tumor antigen is ITGB2. In some embodiments, the first tumor antigen is ITGB3 and the second tumor antigen is GP5. In some embodiments, the first tumor antigen is ITGB3 and the second tumor antigen is GP6. In some embodiments, the first tumor antigen is ITGB3 and the second tumor antigen is GP9. In some embodiments, the first tumor antigen is ITGB3 and the second tumor antigen is GP1BA. In some embodiments, the first tumor antigen is ITGB3 and the second tumor antigen is DSC2. In some embodiments, the first tumor antigen is ITGB3 and the second tumor antigen is FCGR2A. In some embodiments, the first tumor antigen is ITGB3 and the second tumor antigen is TNFRSF10A. In some embodiments, the first tumor antigen is ITGB3 and the second tumor antigen is TNFRSF10B. In some embodiments, the first tumor antigen is ITGB3 and the second tumor antigen is TM4SF1.

In some embodiments of the aforementioned aspects, the first tumor antigen is ITGB2 and the second tumor antigen is CD34. In some embodiments, the first tumor antigen is ITGB2 and the second tumor antigen is CD41. In some embodiments, the first tumor antigen is ITGB2 and the second tumor antigen is G6B. In some embodiments, the first tumor antigen is ITGB2 and the second tumor antigen is P-selectin. In some embodiments, the first tumor antigen is ITGB2 and the second tumor antigen is Clec2. In some embodiments, the first tumor antigen is ITGB2 and the second tumor antigen is cKIT. In some embodiments, the first tumor antigen is ITGB2 and the second tumor antigen is FLT3. In some embodiments, the first tumor antigen is ITGB2 and the second tumor antigen is MPL. In some embodiments, the first tumor antigen is ITGB2 and the second tumor antigen is ITGB3. In some embodiments, the first tumor antigen is ITGB2 and the second tumor antigen is ITGB2. In some embodiments, the first tumor antigen is ITGB2 and the second tumor antigen is GP5. In some embodiments, the first tumor antigen is ITGB2 and the second tumor antigen is GP6. In some embodiments, the first tumor antigen is ITGB2 and the second tumor antigen is GP9. In some embodiments, the first tumor antigen is ITGB2 and the second tumor antigen is GP1BA. In some embodiments, the first tumor antigen is ITGB2 and the second tumor antigen is DSC2. In some embodiments, the first tumor antigen is ITGB2 and the second tumor antigen is FCGR2A. In some embodiments, the first tumor antigen is ITGB2 and the second tumor antigen is TNFRSF10A. In some embodiments, the first tumor antigen is ITGB2 and the second tumor antigen is TNFRSF10B. In some embodiments, the first tumor antigen is ITGB2 and the second tumor antigen is TM4SF1.

In some embodiments of the aforementioned aspects, the first tumor antigen is GP5 and the second tumor antigen is CD34. In some embodiments, the first tumor antigen is GP5 and the second tumor antigen is CD41. In some embodiments, the first tumor antigen is GP5 and the second tumor antigen is G6B. In some embodiments, the first tumor antigen is GP5 and the second tumor antigen is P-selectin. In some embodiments, the first tumor antigen is GP5 and the second tumor antigen is Clec2. In some embodiments, the first tumor antigen is GP5 and the second tumor antigen is cKIT. In some embodiments, the first tumor antigen is GP5 and the second tumor antigen is FLT3. In some embodiments, the first tumor antigen is GP5 and the second tumor antigen is MPL. In some embodiments, the first tumor antigen is GP5 and the second tumor antigen is ITGB3. In some embodiments, the first tumor antigen is GP5 and the second tumor antigen is ITGB2. In some embodiments, the first tumor antigen is GP5 and the second tumor antigen is GP5. In some embodiments, the first tumor antigen is GP5 and the second tumor antigen is GP6. In some embodiments, the first tumor antigen is GP5 and the second tumor antigen is GP9. In some embodiments, the first tumor antigen is GP5 and the second tumor antigen is GP1BA. In some embodiments, the first tumor antigen is GP5 and the second tumor antigen is DSC2. In some embodiments, the first tumor antigen is GP5 and the second tumor antigen is FCGR2A. In some embodiments, the first tumor antigen is GP5 and the second tumor antigen is TNFRSF10A. In some embodiments, the first tumor antigen is GP5 and the second tumor antigen is TNFRSF10B. In some embodiments, the first tumor antigen is GP5 and the second tumor antigen is TM4SF1.

In some embodiments of the aforementioned aspects, the first tumor antigen is GP6 and the second tumor antigen is CD34. In some embodiments, the first tumor antigen is GP6 and the second tumor antigen is CD41. In some embodiments, the first tumor antigen is GP6 and the second tumor antigen is G6B. In some embodiments, the first tumor antigen is GP6 and the second tumor antigen is P-selectin. In some embodiments, the first tumor antigen is GP6 and the second tumor antigen is Clec2. In some embodiments, the first tumor antigen is GP6 and the second tumor antigen is cKIT. In some embodiments, the first tumor antigen is GP6 and the second tumor antigen is FLT3. In some embodiments, the first tumor antigen is GP6 and the second tumor antigen is MPL. In some embodiments, the first tumor antigen is GP6 and the second tumor antigen is ITGB3. In some embodiments, the first tumor antigen is GP6 and the second tumor antigen is ITGB2. In some embodiments, the first tumor antigen is GP6 and the second tumor antigen is GP5. In some embodiments, the first tumor antigen is GP6 and the second tumor antigen is GP6. In some embodiments, the first tumor antigen is GP6 and the second tumor antigen is GP9. In some embodiments, the first tumor antigen is GP6 and the second tumor antigen is GP1BA. In some embodiments, the first tumor antigen is GP6 and the second tumor antigen is DSC2. In some embodiments, the first tumor antigen is GP6 and the second tumor antigen is FCGR2A. In some embodiments, the first tumor antigen is GP6 and the second tumor antigen is TNFRSF10A. In some embodiments, the first tumor antigen is GP6 and the second tumor antigen is TNFRSF10B. In some embodiments, the first tumor antigen is GP6 and the second tumor antigen is TM4SF1.

In some embodiments of the aforementioned aspects, the first tumor antigen is GP9 and the second tumor antigen is CD34. In some embodiments, the first tumor antigen is GP9 and the second tumor antigen is CD41. In some embodiments, the first tumor antigen is GP9 and the second tumor antigen is G6B. In some embodiments, the first tumor antigen is GP9 and the second tumor antigen is P-selectin. In some embodiments, the first tumor antigen is GP9 and the second tumor antigen is Clec2. In some embodiments, the first tumor antigen is GP9 and the second tumor antigen is cKIT. In some embodiments, the first tumor antigen is GP9 and the second tumor antigen is FLT3. In some embodiments, the first tumor antigen is GP9 and the second tumor antigen is MPL. In some embodiments, the first tumor antigen is GP9 and the second tumor antigen is ITGB3. In some embodiments, the first tumor antigen is GP9 and the second tumor antigen is ITGB2. In some embodiments, the first tumor antigen is GP9 and the second tumor antigen is GP5. In some embodiments, the first tumor antigen is GP9 and the second tumor antigen is GP6. In some embodiments, the first tumor antigen is GP9 and the second tumor antigen is GP9. In some embodiments, the first tumor antigen is GP9 and the second tumor antigen is GP1BA. In some embodiments, the first tumor antigen is GP9 and the second tumor antigen is DSC2. In some embodiments, the first tumor antigen is GP9 and the second tumor antigen is FCGR2A. In some embodiments, the first tumor antigen is GP9 and the second tumor antigen is TNFRSF10A. In some embodiments, the first tumor antigen is GP9 and the second tumor antigen is TNFRSF10B. In some embodiments, the first tumor antigen is GP9 and the second tumor antigen is TM4SF1.

In some embodiments of the aforementioned aspects, the first tumor antigen is GP1BA and the second tumor antigen is CD34. In some embodiments, the first tumor antigen is GP1BA and the second tumor antigen is CD41. In some embodiments, the first tumor antigen is GP1BA and the second tumor antigen is G6B. In some embodiments, the first tumor antigen is GP1BA and the second tumor antigen is P-selectin. In some embodiments, the first tumor antigen is GP1BA and the second tumor antigen is Clec2. In some embodiments, the first tumor antigen is GP1BA and the second tumor antigen is cKIT. In some embodiments, the first tumor antigen is GP1BA and the second tumor antigen is FLT3. In some embodiments, the first tumor antigen is GP1BA and the second tumor antigen is MPL. In some embodiments, the first tumor antigen is GP1BA and the second tumor antigen is ITGB3. In some embodiments, the first tumor antigen is GP1BA and the second tumor antigen is ITGB2. In some embodiments, the first tumor antigen is GP1BA and the second tumor antigen is GP5. In some embodiments, the first tumor antigen is GP1BA and the second tumor antigen is GP6. In some embodiments, the first tumor antigen is GP1BA and the second tumor antigen is GP9. In some embodiments, the first tumor antigen is GP1BA and the second tumor antigen is GP1BA. In some embodiments, the first tumor antigen is GP1BA and the second tumor antigen is DSC2. In some embodiments, the first tumor antigen is GP1BA and the second tumor antigen is FCGR2A. In some embodiments, the first tumor antigen is GP1BA and the second tumor antigen is TNFRSF10A. In some embodiments, the first tumor antigen is GP1BA and the second tumor antigen is TNFRSF10B. In some embodiments, the first tumor antigen is GP1BA and the second tumor antigen is TM4SF1.

In some embodiments of the aforementioned aspects, the first tumor antigen is DSC2 and the second tumor antigen is CD34. In some embodiments, the first tumor antigen is DSC2 and the second tumor antigen is CD41. In some embodiments, the first tumor antigen is DSC2 and the second tumor antigen is G6B. In some embodiments, the first tumor antigen is DSC2 and the second tumor antigen is P-selectin. In some embodiments, the first tumor antigen is DSC2 and the second tumor antigen is Clec2. In some embodiments, the first tumor antigen is DSC2 and the second tumor antigen is cKIT. In some embodiments, the first tumor antigen is DSC2 and the second tumor antigen is FLT3. In some embodiments, the first tumor antigen is DSC2 and the second tumor antigen is MPL. In some embodiments, the first tumor antigen is DSC2 and the second tumor antigen is ITGB3. In some embodiments, the first tumor antigen is DSC2 and the second tumor antigen is ITGB2. In some embodiments, the first tumor antigen is DSC2 and the second tumor antigen is GP5. In some embodiments, the first tumor antigen is DSC2 and the second tumor antigen is GP6. In some embodiments, the first tumor antigen is DSC2 and the second tumor antigen is GP9. In some embodiments, the first tumor antigen is DSC2 and the second tumor antigen is GP1BA. In some embodiments, the first tumor antigen is DSC2 and the second tumor antigen is DSC2. In some embodiments, the first tumor antigen is DSC2 and the second tumor antigen is FCGR2A. In some embodiments, the first tumor antigen is DSC2 and the second tumor antigen is TNFRSF10A. In some embodiments, the first tumor antigen is DSC2 and the second tumor antigen is TNFRSF10B. In some embodiments, the first tumor antigen is DSC2 and the second tumor antigen is TM4SF1.

In some embodiments of the aforementioned aspects, the first tumor antigen is FCGR2A and the second tumor antigen is CD34. In some embodiments, the first tumor antigen is FCGR2A and the second tumor antigen is CD41. In some embodiments, the first tumor antigen is FCGR2A and the second tumor antigen is G6B. In some embodiments, the first tumor antigen is FCGR2A and the second tumor antigen is P-selectin. In some embodiments, the first tumor antigen is FCGR2A and the second tumor antigen is Clec2. In some embodiments, the first tumor antigen is FCGR2A and the second tumor antigen is cKIT. In some embodiments, the first tumor antigen is FCGR2A and the second tumor antigen is FLT3. In some embodiments, the first tumor antigen is FCGR2A and the second tumor antigen is MPL. In some embodiments, the first tumor antigen is FCGR2A and the second tumor antigen is ITGB3. In some embodiments, the first tumor antigen is FCGR2A and the second tumor antigen is ITGB2. In some embodiments, the first tumor antigen is FCGR2A and the second tumor antigen is GP5. In some embodiments, the first tumor antigen is FCGR2A and the second tumor antigen is GP6. In some embodiments, the first tumor antigen is FCGR2A and the second tumor antigen is GP9. In some embodiments, the first tumor antigen is FCGR2A and the second tumor antigen is GP1BA. In some embodiments, the first tumor antigen is FCGR2A and the second tumor antigen is DSC2. In some embodiments, the first tumor antigen is FCGR2A and the second tumor antigen is FCGR2A. In some embodiments, the first tumor antigen is FCGR2A and the second tumor antigen is TNFRSF10A. In some embodiments, the first tumor antigen is FCGR2A and the second tumor antigen is TNFRSF10B. In some embodiments, the first tumor antigen is FCGR2A and the second tumor antigen is TM4SF1.

In some embodiments of the aforementioned aspects, the first tumor antigen is TNFRSF10A and the second tumor antigen is CD34. In some embodiments, the first tumor antigen is TNFRSF10A and the second tumor antigen is CD41. In some embodiments, the first tumor antigen is TNFRSF10A and the second tumor antigen is G6B. In some embodiments, the first tumor antigen is TNFRSF10A and the second tumor antigen is P-selectin. In some embodiments, the first tumor antigen is TNFRSF10A and the second tumor antigen is Clec2. In some embodiments, the first tumor antigen is TNFRSF10A and the second tumor antigen is cKIT. In some embodiments, the first tumor antigen is TNFRSF10A and the second tumor antigen is FLT3. In some embodiments, the first tumor antigen is TNFRSF10A and the second tumor antigen is MPL. In some embodiments, the first tumor antigen is TNFRSF10A and the second tumor antigen is ITGB3. In some embodiments, the first tumor antigen is TNFRSF10A and the second tumor antigen is ITGB2. In some embodiments, the first tumor antigen is TNFRSF10A and the second tumor antigen is GP5. In some embodiments, the first tumor antigen is TNFRSF10A and the second tumor antigen is GP6. In some embodiments, the first tumor antigen is TNFRSF10A and the second tumor antigen is GP9. In some embodiments, the first tumor antigen is TNFRSF10A and the second tumor antigen is GP1BA. In some embodiments, the first tumor antigen is TNFRSF10A and the second tumor antigen is DSC2. In some embodiments, the first tumor antigen is TNFRSF10A and the second tumor antigen is FCGR2A. In some embodiments, the first tumor antigen is TNFRSF10A and the second tumor antigen is TNFRSF10A. In some embodiments, the first tumor antigen is TNFRSF10A and the second tumor antigen is TM4SF1.

In some embodiments of the aforementioned aspects, the first tumor antigen is TNFRSF10B and the second tumor antigen is CD34. In some embodiments, the first tumor antigen is TNFRSF10B and the second tumor antigen is CD41. In some embodiments, the first tumor antigen is TNFRSF10B and the second tumor antigen is G6B. In some embodiments, the first tumor antigen is TNFRSF10B and the second tumor antigen is P-selectin. In some embodiments, the first tumor antigen is TNFRSF10B and the second tumor antigen is Clec2. In some embodiments, the first tumor antigen is TNFRSF10B and the second tumor antigen is cKIT. In some embodiments, the first tumor antigen is TNFRSF10B and the second tumor antigen is FLT3. In some embodiments, the first tumor antigen is TNFRSF10B and the second tumor antigen is MPL. In some embodiments, the first tumor antigen is TNFRSF10B and the second tumor antigen is ITGB3. In some embodiments, the first tumor antigen is TNFRSF10B and the second tumor antigen is ITGB2. In some embodiments, the first tumor antigen is TNFRSF10B and the second tumor antigen is GP5. In some embodiments, the first tumor antigen is TNFRSF10B and the second tumor antigen is GP6. In some embodiments, the first tumor antigen is TNFRSF10B and the second tumor antigen is GP9. In some embodiments, the first tumor antigen is TNFRSF10B and the second tumor antigen is GP1BA. In some embodiments, the first tumor antigen is TNFRSF10B and the second tumor antigen is DSC2. In some embodiments, the first tumor antigen is TNFRSF10B and the second tumor antigen is FCGR2A. In some embodiments, the first tumor antigen is TNFRSF10B and the second tumor antigen is TNFRSF10B. In some embodiments, the first tumor antigen is TNFRSF10B and the second tumor antigen is TM4SF1.

In some embodiments of the aforementioned aspects, the first tumor antigen is TM4SF1 and the second tumor antigen is CD34. In some embodiments, the first tumor antigen is TM4SF1 and the second tumor antigen is CD41. In some embodiments, the first tumor antigen is TM4SF1 and the second tumor antigen is G6B. In some embodiments, the first tumor antigen is TM4SF1 and the second tumor antigen is P-selectin. In some embodiments, the first tumor antigen is TM4SF1 and the second tumor antigen is Clec2. In some embodiments, the first tumor antigen is TM4SF1 and the second tumor antigen is cKIT. In some embodiments, the first tumor antigen is TM4SF1 and the second tumor antigen is FLT3. In some embodiments, the first tumor antigen is TM4SF1 and the second tumor antigen is MPL. In some embodiments, the first tumor antigen is TM4SF1 and the second tumor antigen is ITGB3. In some embodiments, the first tumor antigen is TM4SF1 and the second tumor antigen is ITGB2. In some embodiments, the first tumor antigen is TM4SF1 and the second tumor antigen is GP5. In some embodiments, the first tumor antigen is TM4SF1 and the second tumor antigen is GP6. In some embodiments, the first tumor antigen is TM4SF1 and the second tumor antigen is GP9. In some embodiments, the first tumor antigen is TM4SF1 and the second tumor antigen is GP1BA. In some embodiments, the first tumor antigen is TM4SF1 and the second tumor antigen is DSC2. In some embodiments, the first tumor antigen is TM4SF1 and the second tumor antigen is FCGR2A. In some embodiments, the first tumor antigen is TM4SF1 and the second tumor antigen is TNFRSF10A. In some embodiments, the first tumor antigen is TM4SF1 and the second tumor antigen is TNFRSF10B. In some embodiments, the first tumor antigen is TM4SF1 and the second tumor antigen is TM4SF1.

In some embodiments, the first, second, or third tumor antigen is CD34.

In some embodiments, the first, second, or third tumor-targeting moiety comprises a CDR, a framework region, or a variable region sequence disclosed in Table 3 or Table 4 (or a sequence having at least about 75%, 80%, 85%, 90%, 95%, or 99% sequence identity thereto). In some embodiments, the first, second, or third tumor-targeting moiety comprises a HCDR1, HCDR2, HCDR3, LCDR1, LCDR2, and LCDR3 comprising the amino acid sequences of SEQ ID NOs: 87, 88, 89, 90, 91, and 92, respectively (or a sequence with no more than 1, 2, 3, or 4 mutations, e.g., substitutions, additions, or deletions). In some embodiments, the first, second, or third tumor-targeting moiety comprises a VH comprising the amino acid sequence of SEQ ID NO: 79, 80, 81, or 82 (or an amino acid sequence having at least about 80%, 85%, 90%, 95%, or 99% sequence identity thereto). In some embodiments, the first, second, or third tumor-targeting moiety comprises a VL comprising the amino acid sequence of SEQ ID NO: 83, 84, 85, or 86 (or an amino acid sequence having at least about 80%, 85%, 90%, 95%, or 99% sequence identity thereto). In some embodiments, the first, second, or third tumor-targeting moiety comprises a VH comprising any one of SEQ ID NOs: 79, 80, 81, and 82 and a VL comprising any one of SEQ ID NOs: 83, 84, 85, and 86. In some embodiments, the first, second, or third tumor-targeting moiety comprises a VH comprising the amino acid sequence of SEQ ID NO: 79 (or an amino acid sequence having at least about 80%, 85%, 90%, 95%, or 99% sequence identity thereto) and a VL comprising the amino acid sequence of SEQ ID NO: 84 (or an amino acid sequence having at least about 80%, 85%, 90%, 95%, or 99% sequence identity thereto). In some embodiments, the first, second, or third tumor-targeting moiety comprises a VH comprising the amino acid sequence of SEQ ID NO: 79 and a VL comprising the amino acid sequence of SEQ ID NO: 84.

In some embodiments, the first, second, or third tumor-targeting moiety comprises:
(i) a HCDR1, HCDR2, and/or HCDR3 from SEQ ID NO: 1 (or a sequence with no more than 1, 2, 3, or 4 mutations, e.g., substitutions, additions, or deletions),
(ii) a VH of SEQ ID NO: 1 (or a sequence having at least about 75%, 80%, 85%, 90%, 95%, or 99% sequence identity thereto),
(iii) a LCDR1, LCDR2, and/or LCDR3 from SEQ ID NO: 2 (or a sequence with no more than 1, 2, 3, or 4 mutations, e.g., substitutions, additions, or deletions), and/or
(iv) a VL of SEQ ID NO: 2 (or a sequence having at least about 75%, 80%, 85%, 90%, 95%, or 99% sequence identity thereto).

In some embodiments, wherein the first, second, or third tumor antigen is CD41. In some embodiments, the first, second, or third tumor-targeting moiety comprises:
(i) a HCDR1, HCDR2, and/or HCDR3 from SEQ ID NO: 7 (or a sequence with no more than 1, 2, 3, or 4 mutations, e.g., substitutions, additions, or deletions),
(ii) a VH of SEQ ID NO: 7 (or a sequence having at least about 75%, 80%, 85%, 90%, 95%, or 99% sequence identity thereto),
(iii) a LCDR1, LCDR2, and/or LCDR3 from SEQ ID NO: 8 (or a sequence with no more than 1, 2, 3, or 4 mutations, e.g., substitutions, additions, or deletions), and/or
(iv) a VL of SEQ ID NO: 8 (or a sequence having at least about 75%, 80%, 85%, 90%, 95%, or 99% sequence identity thereto).

In some embodiments, the first, second, or third tumor antigen is G6B.

In some embodiments, the first, second, or third tumor antigen is P-selectin. In some embodiments, the first, second, or third tumor-targeting moiety comprises:
(i) a HCDR1, HCDR2, and/or HCDR3 from SEQ ID NO: 13 (or a sequence with no more than 1, 2, 3, or 4 mutations, e.g., substitutions, additions, or deletions),
(ii) a VH of SEQ ID NO: 13 (or a sequence having at least about 75%, 80%, 85%, 90%, 95%, or 99% sequence identity thereto),
(iii) a LCDR1, LCDR2, and/or LCDR3 from SEQ ID NO: 14 (or a sequence with no more than 1, 2, 3, or 4 mutations, e.g., substitutions, additions, or deletions), and/or
(iv) a VL of SEQ ID NO: 14 (or a sequence having at least about 75%, 80%, 85%, 90%, 95%, or 99% sequence identity thereto).

In some embodiments, the first, second, or third tumor antigen is Clec2.

In some embodiments, the first, second, or third tumor antigen is cKIT. In some embodiments, the first, second, or third tumor-targeting moiety comprises:
(i) a HCDR1, HCDR2, and/or HCDR3 from SEQ ID NO: 3 (or a sequence with no more than 1, 2, 3, or 4 mutations, e.g., substitutions, additions, or deletions),
(ii) a VH of SEQ ID NO: 3 (or a sequence having at least about 75%, 80%, 85%, 90%, 95%, or 99% sequence identity thereto),
(iii) a LCDR1, LCDR2, and/or LCDR3 from SEQ ID NO: 4 (or a sequence with no more than 1, 2, 3, or 4 mutations, e.g., substitutions, additions, or deletions), and/or
(iv) a VL of SEQ ID NO: 4 (or a sequence having at least about 75%, 80%, 85%, 90%, 95%, or 99% sequence identity thereto).

In some embodiments, the first, second, or third tumor antigen is FLT3. In some embodiments, the first, second, or third tumor-targeting moiety comprises:
(i) a HCDR1, HCDR2, and/or HCDR3 from SEQ ID NO: 5 (or a sequence with no more than 1, 2, 3, or 4 mutations, e.g., substitutions, additions, or deletions),
(ii) a VH of SEQ ID NO: 5 (or a sequence having at least about 75%, 80%, 85%, 90%, 95%, or 99% sequence identity thereto),
(iii) a LCDR1, LCDR2, and/or LCDR3 from SEQ ID NO: 6 (or a sequence with no more than 1, 2, 3, or 4 mutations, e.g., substitutions, additions, or deletions), and/or
(iv) a VL of SEQ ID NO: 6 (or a sequence having at least about 75%, 80%, 85%, 90%, 95%, or 99% sequence identity thereto).

In some embodiments, the first, second, or third tumor antigen is MPL. In some embodiments, the first, second, or third tumor-targeting moiety comprises:
(i) (a) a HCDR1, HCDR2, and/or HCDR3 from SEQ ID NO: 9 (or a sequence with no more than 1, 2, 3, or 4 mutations, e.g., substitutions, additions, or deletions),
   (b) a VH of SEQ ID NO: 9 (or a sequence having at least about 75%, 80%, 85%, 90%, 95%, or 99% sequence identity thereto),
   (c) a LCDR1, LCDR2, and/or LCDR3 from SEQ ID NO: 10 (or a sequence with no more than 1, 2, 3, or 4 mutations, e.g., substitutions, additions, or deletions), and/or
   (d) a VL of SEQ ID NO: 10 (or a sequence having at least about 75%, 80%, 85%, 90%, 95%, or 99% sequence identity thereto); or
(ii) (a) a HCDR1, HCDR2, and/or HCDR3 from SEQ ID NO: 11 (or a sequence with no more than 1, 2, 3, or 4 mutations, e.g., substitutions, additions, or deletions),
   (b) a VH of SEQ ID NO: 11 (or a sequence having at least about 75%, 80%, 85%, 90%, 95%, or 99% sequence identity thereto),
   (c) a LCDR1, LCDR2, and/or LCDR3 from SEQ ID NO: 12 (or a sequence with no more than 1, 2, 3, or 4 mutations, e.g., substitutions, additions, or deletions), and/or
   (d) a VL of SEQ ID NO: 12 (or a sequence having at least about 75%, 80%, 85%, 90%, 95%, or 99% sequence identity thereto).

In some embodiments, the first, second, or third tumor antigen is ITGB3.

In some embodiments, the first, second, or third tumor antigen is ITGB2.

In some embodiments, the first, second, or third tumor antigen is GP5.

In some embodiments, the first, second, or third tumor antigen is GP6.

In some embodiments, the first, second, or third tumor antigen is GP9.

In some embodiments, the first, second, or third tumor antigen is GP1BA.

In some embodiments, the first, second, or third tumor antigen is DSC2. In some embodiments, the first, second, or third tumor-targeting moiety comprises:
(i) (a) a HCDR1, HCDR2, and/or HCDR3 from SEQ ID NO: 15 (or a sequence with no more than 1, 2, 3, or 4 mutations, e.g., substitutions, additions, or deletions),
   (b) a VH of SEQ ID NO: 15 (or a sequence having at least about 75%, 80%, 85%, 90%, 95%, or 99% sequence identity thereto),
   (c) a LCDR1, LCDR2, and/or LCDR3 from SEQ ID NO: 16 (or a sequence with no more than 1, 2, 3, or 4 mutations, e.g., substitutions, additions, or deletions), and/or
   (d) a VL of SEQ ID NO: 16 (or a sequence having at least about 75%, 80%, 85%, 90%, 95%, or 99% sequence identity thereto); or
(ii) (a) a HCDR1, HCDR2, and/or HCDR3 from SEQ ID NO: 17 (or a sequence with no more than 1, 2, 3, or 4 mutations, e.g., substitutions, additions, or deletions),
   (b) a VH of SEQ ID NO: 17 (or a sequence having at least about 75%, 80%, 85%, 90%, 95%, or 99% sequence identity thereto),
   (c) a LCDR1, LCDR2, and/or LCDR3 from SEQ ID NO: 18 (or a sequence with no more than 1, 2, 3, or 4 mutations, e.g., substitutions, additions, or deletions), and/or
   (d) a VL of SEQ ID NO: 18 (or a sequence having at least about 75%, 80%, 85%, 90%, 95%, or 99% sequence identity thereto).

In some embodiments, the first, second, or third tumor antigen is FCGR2A. In some embodiments, the first, second, or third tumor-targeting moiety comprises:
(i) (a) a HCDR1, HCDR2, and/or HCDR3 from SEQ ID NO: 19 (or a sequence with no more than 1, 2, 3, or 4 mutations, e.g., substitutions, additions, or deletions),
   (b) a VH of SEQ ID NO: 19 (or a sequence having at least about 75%, 80%, 85%, 90%, 95%, or 99% sequence identity thereto),
   (c) a LCDR1, LCDR2, and/or LCDR3 from SEQ ID NO: 20 (or a sequence with no more than 1, 2, 3, or 4 mutations, e.g., substitutions, additions, or deletions), and/or
   (d) a VL of SEQ ID NO: 20 (or a sequence having at least about 75%, 80%, 85%, 90%, 95%, or 99% sequence identity thereto);
(ii) (a) a HCDR1, HCDR2, and/or HCDR3 from SEQ ID NO: 21 (or a sequence with no more than 1, 2, 3, or 4 mutations, e.g., substitutions, additions, or deletions),
   (b) a VH of SEQ ID NO: 21 (or a sequence having at least about 75%, 80%, 85%, 90%, 95%, or 99% sequence identity thereto),
   (c) a LCDR1, LCDR2, and/or LCDR3 from SEQ ID NO: 22 (or a sequence with no more than 1, 2, 3, or 4 mutations, e.g., substitutions, additions, or deletions), and/or
   (d) a VL of SEQ ID NO: 22 (or a sequence having at least about 75%, 80%, 85%, 90%, 95%, or 99% sequence identity thereto), or
(iii) (a) a HCDR1, HCDR2, and/or HCDR3 from SEQ ID NO: 23 (or a sequence with no more than 1, 2, 3, or 4 mutations, e.g., substitutions, additions, or deletions),
   (b) a VH of SEQ ID NO: 23 (or a sequence having at least about 75%, 80%, 85%, 90%, 95%, or 99% sequence identity thereto),
   (c) a LCDR1, LCDR2, and/or LCDR3 from SEQ ID NO: 24 (or a sequence with no more than 1, 2, 3, or 4 mutations, e.g., substitutions, additions, or deletions), and/or
   (d) a VL of SEQ ID NO: 24 (or a sequence having at least about 75%, 80%, 85%, 90%, 95%, or 99% sequence identity thereto).

In some embodiments, the first, second, or third tumor antigen is TNFRSF10A or TNFRSF10B. In some embodiments, the first, second, or third tumor-targeting moiety comprises:
(i) (a) a HCDR1, HCDR2, and/or HCDR3 from SEQ ID NO: 25 (or a sequence with no more than 1, 2, 3, or 4 mutations, e.g., substitutions, additions, or deletions),
   (b) a VH of SEQ ID NO: 25 (or a sequence having at least about 75%, 80%, 85%, 90%, 95%, or 99% sequence identity thereto),
   (c) a LCDR1, LCDR2, and/or LCDR3 from SEQ ID NO: 26 (or a sequence with no more than 1, 2, 3, or 4 mutations, e.g., substitutions, additions, or deletions), and/or
   (d) a VL of SEQ ID NO: 26 (or a sequence having at least about 75%, 80%, 85%, 90%, 95%, or 99% sequence identity thereto); or
(ii) (a) a HCDR1, HCDR2, and/or HCDR3 from SEQ ID NO: 27 (or a sequence with no more than 1, 2, 3, or 4 mutations, e.g., substitutions, additions, or deletions),
   (b) a VH of SEQ ID NO: 27 (or a sequence having at least about 75%, 80%, 85%, 90%, 95%, or 99% sequence identity thereto),
   (c) a LCDR1, LCDR2, and/or LCDR3 from SEQ ID NO: 28 (or a sequence with no more than 1, 2, 3, or 4 mutations, e.g., substitutions, additions, or deletions), and/or
   (d) a VL of SEQ ID NO: 28 (or a sequence having at least about 75%, 80%, 85%, 90%, 95%, or 99% sequence identity thereto);
(iii) (a) a HCDR1, HCDR2, and/or HCDR3 from SEQ ID NO: 29 (or a sequence with no more than 1, 2, 3, or 4 mutations, e.g., substitutions, additions, or deletions),
   (b) a VH of SEQ ID NO: 29 (or a sequence having at least about 75%, 80%, 85%, 90%, 95%, or 99% sequence identity thereto),
   (c) a LCDR1, LCDR2, and/or LCDR3 from SEQ ID NO: 30 (or a sequence with no more than 1, 2, 3, or 4 mutations, e.g., substitutions, additions, or deletions), and/or
   (d) a VL of SEQ ID NO: 30 (or a sequence having at least about 75%, 80%, 85%, 90%, 95%, or 99% sequence identity thereto);
(iv) (a) a HCDR1, HCDR2, and/or HCDR3 from SEQ ID NO: 31 (or a sequence with no more than 1, 2, 3, or 4 mutations, e.g., substitutions, additions, or deletions),
   (b) a VH of SEQ ID NO: 31 (or a sequence having at least about 75%, 80%, 85%, 90%, 95%, or 99% sequence identity thereto),
   (c) a LCDR1, LCDR2, and/or LCDR3 from SEQ ID NO: 32 (or a sequence with no more than 1, 2, 3, or 4 mutations, e.g., substitutions, additions, or deletions), and/or
   (d) a VL of SEQ ID NO: 32 (or a sequence having at least about 75%, 80%, 85%, 90%, 95%, or 99% sequence identity thereto); or
(v) (a) a HCDR1, HCDR2, and/or HCDR3 from SEQ ID NO: 33 (or a sequence with no more than 1, 2, 3, or 4 mutations, e.g., substitutions, additions, or deletions),
   (b) a VH of SEQ ID NO: 33 (or a sequence having at least about 75%, 80%, 85%, 90%, 95%, or 99% sequence identity thereto),
   (c) a LCDR1, LCDR2, and/or LCDR3 from SEQ ID NO: 34 (or a sequence with no more than 1, 2, 3, or 4 mutations, e.g., substitutions, additions, or deletions), and/or
   (d) a VL of SEQ ID NO: 34 (or a sequence having at least about 75%, 80%, 85%, 90%, 95%, or 99% sequence identity thereto).

In some embodiments, the first, second, or third tumor antigen is TM4SF1. In some embodiments, the first, second, or third tumor-targeting moiety comprises:
(i) a HCDR1, HCDR2, and/or HCDR3 from SEQ ID NO: 35 (or a sequence with no more than 1, 2, 3, or 4 mutations, e.g., substitutions, additions, or deletions),
(ii) a VH of SEQ ID NO: 35 (or a sequence having at least about 75%, 80%, 85%, 90%, 95%, or 99% sequence identity thereto),
(iii) a LCDR1, LCDR2, and/or LCDR3 from SEQ ID NO: 36 (or a sequence with no more than 1, 2, 3, or 4 mutations, e.g., substitutions, additions, or deletions), and/or
(iv) a VL of SEQ ID NO: 36 (or a sequence having at least about 75%, 80%, 85%, 90%, 95%, or 99% sequence identity thereto).

In some embodiments, the multifunctional molecule comprises one of the immune cell engager, the cytokine molecule (or the modulator of a cytokine molecule), and the stromal modifying moiety. In some embodiments, the multifunctional molecule comprises the first tumor-targeting moiety, the second tumor-targeting moiety, the immune cell engager, and optionally the third tumor-targeting moiety. In some embodiments, the multifunctional molecule comprises the first tumor-targeting moiety, the second tumor-targeting moiety, the cytokine molecule (or the modulator of a cytokine molecule), and optionally the third tumor-targeting moiety. In some embodiments, the multifunctional molecule comprises the first tumor-targeting moiety, the second tumor-targeting moiety, the stromal modifying moiety, and optionally the third tumor-targeting moiety.

In some embodiments, the multifunctional molecule comprises two of the immune cell engager, the cytokine molecule (or the modulator of a cytokine molecule), and the stromal modifying moiety. In some embodiments, the multifunctional molecule comprises the first tumor-targeting moiety, the second tumor-targeting moiety, the immune cell engager, the cytokine molecule (or the modulator of a cytokine molecule), and optionally the third tumor-targeting moiety. In some embodiments, the multifunctional molecule comprises the first tumor-targeting moiety, the second tumor-targeting moiety, the immune cell engager, the stromal modifying moiety, and optionally the third tumor-targeting moiety. In some embodiments, the multifunctional molecule comprises the first tumor-targeting moiety, the second tumor-targeting moiety, the cytokine molecule (or the modulator of a cytokine molecule), the stromal modifying moiety, and optionally the third tumor-targeting moiety.

In some embodiments, the multifunctional molecule comprises all of the immune cell engager, the cytokine molecule (or the modulator of a cytokine molecule), and the stromal modifying moiety. In some embodiments, the multifunctional molecule comprises the first tumor-targeting moiety, the second tumor-targeting moiety, the immune cell engager, the cytokine molecule (or the modulator of a cytokine molecule), the stromal modifying moiety, and optionally the third tumor-targeting moiety.

In some embodiments, the multifunctional molecule comprises an immune cell engager chosen from a T cell engager, an NK cell engager, a B cell engager, a dendritic cell engager, or a macrophage cell engager. In some embodiments, the immune cell engager binds to and activates an immune cell, e.g., an effector cell. In some embodiments, the immune cell engager binds to, but does not activate, an immune cell, e.g., an effector cell.

In some embodiments, the immune cell engager is a T cell engager, e.g., a T cell engager that mediates binding to and activation of a T cell, or a T cell engager that mediates binding to but not activation of a T cell. In some embodiments, the T cell engager binds to CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, or CD226, e.g., the T cell engager is an anti-CD3 antibody molecule.

In some embodiments, the immune cell engager is an NK cell engager, e.g., an NK cell engager that mediates binding to and activation of an NK cell, or an NK cell engager that mediates binding to but not activation of an NK cell. In some embodiments, the NK cell engager is chosen from an antibody molecule, e.g., an antigen binding domain, or ligand that binds to (e.g., activates): NKp30, NKp40, NKp44, NKp46, NKG2D, DNAM1, DAP10, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, or CD160. In some embodiments, the NK cell engager is an antibody molecule, e.g., an antigen binding domain.

In some embodiments, the NK cell engager is an antibody molecule, e.g., an antigen binding domain, that binds to NKp30 or NKp46. In some embodiments, the NK cell engager is a ligand, optionally, the ligand further comprises an immunoglobulin constant region, e.g., an Fc region. In some embodiments, the NK cell engager is a ligand of NKp44 or NKp46, e.g., a viral HA. In some embodiments, the NK cell engager is a ligand of DAP10, e.g., a coreceptor for NKG2D. In some embodiments, the NK cell engager is a ligand of CD16, e.g., a CD16a/b ligand, e.g., a CD16a/b ligand further comprising an antibody Fc region.

In some embodiments, the immune cell engager mediates binding to, or activation of, or both of, one or more of a B cell, a macrophage, and/or a dendritic cell. In some embodiments, the immune cell engager comprises a B cell, macrophage, and/or dendritic cell engager chosen from one or more of CD40 ligand (CD40L) or a CD70 ligand; an antibody molecule that binds to CD40 or CD70; an antibody molecule to OX40; an OX40 ligand (OX40L); an agonist of a Toll-like receptor (e.g., a TLR4, e.g., a constitutively active TLR4 (caTLR4) or a TLR9 agonist); a 41BB; a CD2 agonist; a CD47; or a STING agonist, or a combination thereof.

In some embodiments, the immune cell engager is a B cell engager, e.g., a CD40L, an OX40L, or a CD70 ligand, or an antibody molecule that binds to OX40, CD40 or CD70.

In some embodiments, the immune cell engager is a macrophage cell engager, e.g., a CD2 agonist; a CD40L; an OX40L; an antibody molecule that binds to OX40, CD40 or CD70; an agonist of a Toll-like receptor (TLR) (e.g., a TLR4, e.g., a constitutively active TLR4 (caTLR4) or a TLR9 agonist); CD47; or a STING agonist.

In some embodiments, the immune cell engager is a dendritic cell engager, e.g., a CD2 agonist, an OX40 antibody, an OX40L, 41BB agonist, a Toll-like receptor agonist or a fragment thereof (e.g., a TLR4, e.g., a constitutively active TLR4 (caTLR4)), CD47 agonist, or a STING agonist.

In some embodiments, the STING agonist comprises a cyclic dinucleotide, e.g., a cyclic di-GMP (cdGMP), a cyclic di-AMP (cdAMP), or a combination thereof, optionally with 2′,5′ or 3′,5′ phosphate linkages, e.g., wherein the STING agonist is covalently coupled to the multifunctional molecule.

In some embodiments, the multifunctional molecule comprises a cytokine molecule.

In some embodiments, the cytokine molecule is chosen from interleukin-2 (IL-2), interleukin-7 (IL-7), interleukin-12 (IL-12), interleukin-15 (IL-15), interleukin-18 (IL-18), interleukin-21 (IL-21), or interferon gamma, or a fragment or variant thereof, or a combination of any of the aforesaid cytokines. In some embodiments, the cytokine molecule is a monomer or a dimer. In some embodiments, the cytokine molecule further comprises a receptor dimerizing domain, e.g., an IL15Ralpha dimerizing domain. In some embodiments, the cytokine molecule (e.g., IL-15) and the receptor dimerizing domain (e.g., an IL15Ralpha dimerizing domain) are not covalently linked, e.g., are non-covalently associated.

In some embodiments, the multifunctional molecule comprises a modulator of a cytokine molecule. In some embodiments, the modulator of a cytokine molecule is a TGF-beta inhibitor disclosed herein. In some embodiments, the TGF-beta inhibitor comprises a portion of a TGF-beta receptor (e.g., an extracellular domain of a TGF-beta receptor) that is capable of inhibiting (e.g., reducing the activity of) TGF-beta, or functional fragment or variant thereof. In some embodiments, the TGF-beta inhibitor comprises an extracellular domain of TGFBR1 or a sequence substantially identical thereto (e.g., a sequence that is at least 80%, 85%, 90%, or 95% identical thereto). In some embodiments, the TGF-beta inhibitor comprises an extracellular domain of TGFBR2 or a sequence substantially identical thereto (e.g., a sequence that is at least 80%, 85%, 90%, or 95% identical thereto). In some embodiments, the TGF-beta inhibitor comprises an extracellular domain of TGFBR3 or a sequence substantially identical thereto (e.g., a sequence that is at least 80%, 85%, 90%, or 95% identical thereto). In some embodiments, the TGF-beta inhibitor comprises an amino acid sequence disclosed in Table 12 or a sequence that is at least 80%, 85%, 90%, or 95% identical thereto.

In some embodiments, the multifunctional molecule comprises a stromal modifying moiety. In some embodiments, the stromal modifying moiety causes one or more of: decreases the level or production of a stromal or extracellular matrix (ECM) component; decreases tumor fibrosis; increases interstitial tumor transport; improves tumor perfusion; expands the tumor microvasculature; decreases interstitial fluid pressure (IFP) in a tumor; or decreases or enhances penetration or diffusion of an agent, e.g., a cancer therapeutic or a cellular therapy, into a tumor or tumor vasculature. In some embodiments, the stromal or ECM component decreased is chosen from a glycosaminoglycan or an extracellular protein, or a combination thereof. In some embodiments, the glycosaminoglycan is chosen from hyaluronan (also known as hyaluronic acid or HA), chondroitin sulfate, chondroitin, dermatan sulfate, heparan sulfate, heparin, entactin, tenascin, aggrecan or keratin sulfate. In some embodiments, the extracellular protein is chosen from collagen, laminin, elastin, fibrinogen, fibronectin, or vitronectin. In some embodiments, the stromal modifying moiety comprises an enzyme molecule that degrades a tumor stroma or extracellular matrix (ECM). In some embodiments, the enzyme molecule is chosen from a hyaluronidase molecule, a collagenase molecule, a chondroitinase molecule, a matrix metalloproteinase molecule (e.g., macrophage metalloelastase), or a variant (e.g., a fragment) of any of the aforesaid. In some embodiments, the stromal modifying moiety decreases the level or production of hyaluronic acid. In some embodiments, the stromal modifying moiety comprises a hyaluronan degrading enzyme, an agent that inhibits hyaluronan synthesis, or an antibody molecule against hyaluronic acid. In some embodiments, the hyaluronan degrading enzyme is a hyaluronidase molecule or a variant (e.g., fragment thereof) thereof. In some embodiments, the hyaluronan degrading enzyme is active in neutral or acidic pH, e.g., pH of about 4-5. In some embodiments, the hyaluronidase molecule is a mammalian hyaluronidase molecule, e.g., a recombinant human hyaluronidase molecule, or a variant thereof (e.g., a truncated form thereof). In some embodiments, the hyaluronidase molecule is chosen from HYAL1, HYAL2, or PH-20/SPAM1, or a variant thereof (e.g., a truncated form thereof). In some embodiments, the truncated form lacks a C-terminal glycosylphosphatidylinositol (GPI) attachment site or a portion of the GPI attachment site. In some embodiments, the hyaluronidase molecule is glycosylated, e.g., comprises at least one N-linked glycan. In some embodiments, the hyaluronidase molecule comprises the amino acid sequence of: SEQ ID NO: 66, or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 66). In some embodiments, the hyaluronidase molecule comprises:
(i) the amino acid residues 36-464 of SEQ ID NO: 66;
(ii) the amino acid residues 36-481, 36-482, or 36-483 of PH20, wherein PH20 has the amino acid sequence of SEQ ID NO: 66; or
(iii) an amino acid sequence having at least 95% to 100% sequence identity to the polypeptide or truncated form of the amino acid sequence of SEQ ID NO: 66; or
(iv) an amino acid sequence having 30, 20, 10, 5 or fewer amino acid substitutions to the amino acid sequence of SEQ ID NO: 66.

In some embodiments, the hyaluronidase molecule comprises an amino acid sequence at least 95% (e.g., at least 95%, 96%, 97%, 98%, 99%, 100%) identical to the amino acid sequence of SEQ ID NO: 66, or the hyaluronidase molecule is encoded by a nucleotide sequence at least 95% (e.g., at least 96%, 97%, 98%, 99%, 100%) identical to the nucleotide sequence of SEQ ID NO: 66.

In some embodiments, the hyaluronidase molecule is PH20, e.g., rHuPH20.

In some embodiments, the hyaluronidase molecule is HYAL1 and comprises the amino acid sequence of: SEQ ID NO: 67, or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 67).

In some embodiments, the hyaluronan degrading enzyme, e.g., the hyaluronidase molecule, further comprises a polymer, e.g., is conjugated to a polymer, e.g., PEG.

In some embodiments, the hyaluronan-degrading enzyme is a PEGylated PH20 enzyme (PEGPH20).

In some embodiments, the hyaluronan degrading enzyme, e.g., the hyaluronidase molecule, further comprises an immunoglobulin chain constant region (e.g., Fc region) chosen from, e.g., the heavy chain constant regions of IgG1, IgG2, IgG3, or IgG4, more particularly, the heavy chain constant region of human IgG1, IgG2, IgG3, or IgG4.

In some embodiments, the immunoglobulin constant region (e.g., the Fc region) is linked, e.g., covalently linked to, the hyaluronan degrading enzyme, e.g., the hyaluronidase molecule.

In some embodiments, the immunoglobulin chain constant region (e.g., Fc region) is altered, e.g., mutated, to increase or decrease one or more of: Fc receptor binding, antibody glycosylation, the number of cysteine residues, effector cell function, or complement function.

In some embodiments, the hyaluronan degrading enzyme, e.g., the hyaluronidase molecule, forms a dimer.

In some embodiments, the stromal modifying moiety comprises an inhibitor of the synthesis of hyaluronan, e.g., an HA synthase. In some embodiments, the inhibitor comprises a sense or an antisense nucleic acid molecule against an HA synthase or is a small molecule drug, optionally wherein the inhibitor is 4-methylumbelliferone (MU) or a derivative thereof (e.g., 6,7-dihydroxy-4-methyl coumarin or 5,7-dihydroxy-4-methyl coumarin), or leflunomide or a derivative thereof.

In some embodiments, the stromal modifying moiety comprises a collagenase molecule, e.g., a mammalian collagenase molecule, or a variant (e.g., fragment) thereof.

In some embodiments, the collagenase molecule is collagenase molecule IV, e.g., comprising the amino acid sequence of: SEQ ID NO: 68, or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 68.

In some embodiments, the multifunctional molecule comprises at least two non-contiguous polypeptide chains.

In some embodiments, the multifunctional molecule comprises the following configuration:
A, B-[dimerization module]-C, -D
e.g., the configuration shown in FIGS. 1A, 1B, and 1C, wherein:
(1) the dimerization module comprises an immunoglobulin constant domain, e.g., a heavy chain constant domain (e.g., a homodimeric or heterodimeric heavy chain constant region, e.g., an Fc region), or a constant domain of an immunoglobulin variable region (e.g., a Fab region); and
(2) A, B, C, and D are independently: (a) absent; (b) the first tumor-targeting moiety; (c) the second tumor-targeting moiety; (d) the third tumor-targeting moiety; (e) the immune cell engager; (f) the cytokine molecule (or the modulator of a cytokine molecule); or (g) the stromal modifying moiety.

In some embodiments,
(i) A comprises the first tumor-targeting moiety, B comprises the second tumor-targeting moiety, C comprises a first immune cell engager, and D comprises a second immune cell engager (e.g., C and D comprise the same or different immune cell engagers);
(ii) A comprises the first tumor-targeting moiety, B comprises the second tumor-targeting moiety, C comprises a first cytokine molecule (or a first modulator of a cytokine molecule), and D comprises a second cytokine molecule (or a second modulator of a cytokine molecule) (e.g., C and D comprise the same or different cytokine molecules (or C and D comprise the same or different modulators of a cytokine molecule));
(iii) A comprises the first tumor-targeting moiety, B comprises the second tumor-targeting moiety, C comprises a first stromal modifying moiety, and D comprises a second stromal modifying moiety (e.g., C and D comprise the same or different stromal modifying moieties);
(iv) A comprises the first tumor-targeting moiety, B comprises the second tumor-targeting moiety, C comprises the immune cell engager, and D comprises the cytokine molecule (or the modulator of a cytokine molecule);
(v) A comprises the first tumor-targeting moiety, B comprises the second tumor-targeting moiety, C comprises the cytokine molecule (or the modulator of a cytokine molecule), and D comprises the immune cell engager;
(vi) A comprises the first tumor-targeting moiety, B comprises the second tumor-targeting moiety, C comprises the immune cell engager, and D comprises the stromal modifying moiety;
(vii) A comprises the first tumor-targeting moiety, B comprises the second tumor-targeting moiety, C comprises the stromal modifying moiety, and D comprises the immune cell engager;
(viii) A comprises the first tumor-targeting moiety, B comprises the second tumor-targeting moiety, C comprises the cytokine molecule (or the modulator of a cytokine molecule), and D comprises the stromal modifying moiety;
(ix) A comprises the first tumor-targeting moiety, B comprises the second tumor-targeting moiety, C comprises the stromal modifying moiety, and D comprises the cytokine molecule (or the modulator of a cytokine molecule);
(x) A comprises the first tumor-targeting moiety, B comprises the second tumor-targeting moiety, C comprises the immune cell engager, and D is absent;
(xi) A comprises the first tumor-targeting moiety, B comprises the second tumor-targeting moiety, C is absent, and D comprises the immune cell engager;
(xii) A comprises the first tumor-targeting moiety, B comprises the second tumor-targeting moiety, C comprises the cytokine molecule (or the modulator of a cytokine molecule), and D is absent;
(xiii) A comprises the first tumor-targeting moiety, B comprises the second tumor-targeting moiety, C is absent, and D comprises the cytokine molecule (or the modulator of a cytokine molecule);
(xiv) A comprises the first tumor-targeting moiety, B comprises the second tumor-targeting moiety, C comprises the stromal modifying moiety, and D is absent;
(xv) A comprises the first tumor-targeting moiety, B comprises the second tumor-targeting moiety, C is absent, and D comprises the stromal modifying moiety;
(xvi) A comprises the first tumor-targeting moiety, B comprises the second tumor-targeting moiety, C comprises the third tumor-targeting moiety, and D comprises the immune cell engager;
(xvii) A comprises the first tumor-targeting moiety, B comprises the second tumor-targeting moiety, C comprises the third tumor-targeting moiety, and D comprises the cytokine molecule (or the modulator of a cytokine molecule); or
(xviii) A comprises the first tumor-targeting moiety, B comprises the second tumor-targeting moiety, C comprises the third tumor-targeting moiety, and D comprises a stromal modifying moiety.

In some embodiments, the dimerization module comprises one or more immunoglobulin chain constant regions (e.g., Fc regions) comprising one or more of: a paired cavity-protuberance (“knob-in-a hole”), an electrostatic interaction, or a strand-exchange. In some embodiments, the one or more immunoglobulin chain constant regions (e.g., Fc regions) comprise an amino acid substitution at a position chosen from one or more of 347, 349, 350, 351, 366, 368, 370, 392, 394, 395, 397, 398, 399, 405, 407, or 409, e.g., of the Fc region of human IgG1, optionally wherein the one or more immunoglobulin chain constant regions (e.g., Fc regions) comprise an amino acid substitution chosen from: T366S, L368A, or Y407V (e.g., corresponding to a cavity or hole), or T366W (e.g., corresponding to a protuberance or knob), or a combination thereof.

In some embodiments, the multifunctional molecule further comprises a linker, e.g., a linker between one or more of: the antigen binding domain and the immune cell engager, the antigen binding domain and the cytokine molecule (or the modulator of a cytokine molecule), the antigen binding domain and the stromal modifying moiety, the immune cell engager and the cytokine molecule (or the modulator of a cytokine molecule), the immune cell engager and the stromal modifying moiety, the cytokine molecule (or the modulator of a cytokine molecule) and the stromal modifying moiety, the antigen binding domain and the dimerization module, the immune cell engager and the dimerization module, the cytokine molecule (or the modulator of a cytokine molecule) and the dimerization module, or the stromal modifying moiety and the dimerization module. In some embodiments, the linker is chosen from: a cleavable linker, a non-cleavable linker, a peptide linker, a flexible linker, a rigid linker, a helical linker, or a non-helical linker. In some embodiments, the linker is a peptide linker. In some embodiments, the peptide linker comprises Gly and Ser. In some embodiments, the peptide linker comprises an amino acid sequence chosen from SEQ ID NOs: 69-76.

In one aspect, disclosed herein is a multifunctional molecule, comprising:
(i) a first tumor-targeting moiety that binds to a first tumor antigen, and
(ii) a second tumor-targeting moiety that binds to a second tumor antigen, wherein:
the first and second tumor antigens are each independently chosen from: CD34, CD41, G6B, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1. In some embodiments, the first tumor antigen is different from the second tumor antigen.

In some embodiments, the multifunctional molecule further comprises (iii) a third tumor-targeting moiety that binds to a third tumor antigen. In some embodiments, the third tumor antigen is chosen from: CD34, CD41, G6B, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1.

In some embodiments, the third tumor antigen is different from the first or second tumor antigen. In some embodiments, the first and second tumor antigens are present on the same tumor cell. In some embodiments, the first and third tumor antigens are present on the same tumor cell. In some embodiments, the second and third tumor antigens are present on the same tumor cell. In some embodiments, the first, second, and third tumor antigens are present on the same tumor cell.

In some embodiments, the first and second tumor antigens are present on different tumor cells. In some embodiments, the first and third tumor antigens are present on different tumor cells. In some embodiments, the second and third tumor antigens are present on different tumor cells. In some embodiments, the first, second, and third tumor antigens are present on different tumor cells.

In some embodiments, the first, second, and/or third tumor antigens show higher expression in a tumor cell, e.g., a myeloproliferative neoplasm cell, than a non-tumor cell. In some embodiments, the expression of the first, second, and/or third tumor antigens in a tumor cell, e.g., a myeloproliferative neoplasm cell, is at least 1.5, 2, 4, 6, 8, or 10-fold higher than the expression of the first, second, and/or third tumor antigens in a non-tumor cell. In some embodiments, the multifunctional molecule preferentially binds to a tumor cell, e.g., a myeloproliferative neoplasm cell, over a non-tumor cell. In some embodiments, the binding between the multifunctional molecule and the tumor cell, e.g., a myeloproliferative neoplasm cell, is more than 10, 20, 30, 40, 50-fold greater than the binding between the multifunctional molecule and a non-tumor cell.

In some embodiments, the affinity, e.g., the combined affinity, of the first and second tumor-targeting moieties for a tumor cell, e.g., a myeloproliferative neoplasm cell, is greater than the affinity of a similar multifunctional molecule having only one of the first tumor-targeting moiety or the second tumor-targeting moiety. In some embodiments, the affinity, e.g., the combined affinity, of the first and second tumor-targeting moieties for a tumor cell, e.g., a myeloproliferative neoplasm cell, is at least 2, 5, 10, 20, 30, 40, 50, 75 or 100 times greater than the affinity of a similar multifunctional molecule having only one of the first tumor-targeting moiety or the second tumor-targeting moiety.

In some embodiments, the affinity, e.g., the combined affinity, of the first, second, and third tumor-targeting moieties for a tumor cell, e.g., a myeloproliferative neoplasm cell, is greater than the affinity of a similar multifunctional molecule having only one of the first tumor-targeting moiety, the second tumor-targeting moiety, or the third tumor-targeting moiety, or a similar multifunctional molecule having only two of the first tumor-targeting moiety, the second tumor-targeting moiety, or the third tumor-targeting moiety. In some embodiments, the affinity, e.g., the combined affinity, of the first, second, and third tumor-targeting moieties for a tumor cell, e.g., a myeloproliferative neoplasm cell, is at least 2, 5, 10, 20, 30, 40, 50, 75 or 100 times greater than the affinity of a similar multifunctional molecule having only one of the first tumor-targeting moiety, the second tumor-targeting moiety, or the third tumor-targeting moiety, or a similar multifunctional molecule having only two of the first tumor-targeting moiety, the second tumor-targeting moiety, or the third tumor-targeting moiety.

In some embodiments, the myeloproliferative neoplasm cell is chosen from a myelofibrosis cell, an essential thrombocythemia cell, a polycythemia vera cell, or a chronic myeloid cancer cell. In some embodiments, the myeloproliferative neoplasm cell is a myelofibrosis cell. In some embodiments, the myeloproliferative neoplasm cell is an essential thrombocythemia cell. In some embodiments, the myeloproliferative neoplasm cell is a polycythemia vera cell. In some embodiments, the myeloproliferative neoplasm cell is a chronic myeloid cancer cell. In some embodiments, the myeloproliferative neoplasm cell comprises a JAK2 mutation (e.g., a JAK2 V617F mutation). In some embodiments, the myeloproliferative neoplasm cell comprises a calreticulin mutation. In some embodiments, the myeloproliferative neoplasm cell comprises a MPL mutation.

In some embodiments, the first tumor antigen is CD34 and the second tumor antigen is CD41. In some embodiments, the first tumor antigen is CD34 and the second tumor antigen is G6B. In some embodiments, the first tumor antigen is CD41 and the second tumor antigen is G6B. In some embodiments, the first tumor antigen is CD34, the second tumor antigen is CD41, and the third tumor antigen is G6B.

In some embodiments, the first tumor antigen is P-selectin and the second tumor antigen is Clec2. In some embodiments, the first tumor antigen is CD34 and the second tumor antigen is P-selectin. In some embodiments, the first tumor antigen is CD41 and the second tumor antigen is P-selectin. In some embodiments, the first tumor antigen is G6B and the second tumor antigen is P-selectin. In some embodiments, the first tumor antigen is CD34 and the second tumor antigen is Clec2. In some embodiments, the first tumor antigen is CD41 and the second tumor antigen is Clec2. In some embodiments, the first tumor antigen is G6B and the second tumor antigen is Clec2. In some embodiments, the first tumor antigen is CD34, the second tumor antigen is CD41, and the third tumor antigen is P-selectin. In some embodiments, the first tumor antigen is CD34, the second tumor antigen is G6B, and the third tumor antigen is P-selectin. In some embodiments, the first tumor antigen is CD41, the second tumor antigen is G6B, and the third tumor antigen is P-selectin. In some embodiments, the first tumor antigen is CD34, the second tumor antigen is CD41, and the third tumor antigen is Clec2. In some embodiments, the first tumor antigen is CD34, the second tumor antigen is G6B, and the third tumor antigen is Clec2. In some embodiments, the first tumor antigen is CD41, the second tumor antigen is G6B, and the third tumor antigen is Clec2. In some embodiments, the first tumor antigen is CD34, the second tumor antigen is P-selectin, and the third tumor antigen is Clec2. In some embodiments, the first tumor antigen is CD41, the second tumor antigen is P-selectin, and the third tumor antigen is Clec2. In some embodiments, the first tumor antigen is G6B, the second tumor antigen is P-selectin, and the third tumor antigen is Clec2.

In some embodiments, the first, second, or third tumor antigen is CD34. In some embodiments, the first, second, or third tumor-targeting moiety comprises:
(i) a HCDR1, HCDR2, and/or HCDR3 from SEQ ID NO: 1 (or a sequence with no more than 1, 2, 3, or 4 mutations, e.g., substitutions, additions, or deletions),
(ii) a VH of SEQ ID NO: 1 (or a sequence having at least about 75%, 80%, 85%, 90%, 95%, or 99% sequence identity thereto),
(iii) a LCDR1, LCDR2, and/or LCDR3 from SEQ ID NO: 2 (or a sequence with no more than 1, 2, 3, or 4 mutations, e.g., substitutions, additions, or deletions), and/or
(iv) a VL of SEQ ID NO: 2 (or a sequence having at least about 75%, 80%, 85%, 90%, 95%, or 99% sequence identity thereto).

In some embodiments, wherein the first, second, or third tumor antigen is CD41. In some embodiments, the first, second, or third tumor-targeting moiety comprises:
(i) a HCDR1, HCDR2, and/or HCDR3 from SEQ ID NO: 7 (or a sequence with no more than 1, 2, 3, or 4 mutations, e.g., substitutions, additions, or deletions),
(ii) a VH of SEQ ID NO: 7 (or a sequence having at least about 75%, 80%, 85%, 90%, 95%, or 99% sequence identity thereto),
(iii) a LCDR1, LCDR2, and/or LCDR3 from SEQ ID NO: 8 (or a sequence with no more than 1, 2, 3, or 4 mutations, e.g., substitutions, additions, or deletions), and/or
(iv) a VL of SEQ ID NO: 8 (or a sequence having at least about 75%, 80%, 85%, 90%, 95%, or 99% sequence identity thereto).

In some embodiments, the first, second, or third tumor antigen is G6B.

In some embodiments, the first, second, or third tumor antigen is P-selectin. In some embodiments, the first, second, or third tumor-targeting moiety comprises:
(i) a HCDR1, HCDR2, and/or HCDR3 from SEQ ID NO: 13 (or a sequence with no more than 1, 2, 3, or 4 mutations, e.g., substitutions, additions, or deletions),
(ii) a VH of SEQ ID NO: 13 (or a sequence having at least about 75%, 80%, 85%, 90%, 95%, or 99% sequence identity thereto),
(iii) a LCDR1, LCDR2, and/or LCDR3 from SEQ ID NO: 14 (or a sequence with no more than 1, 2, 3, or 4 mutations, e.g., substitutions, additions, or deletions), and/or
(iv) a VL of SEQ ID NO: 14 (or a sequence having at least about 75%, 80%, 85%, 90%, 95%, or 99% sequence identity thereto).

In some embodiments, the first, second, or third tumor antigen is Clec2.

In some embodiments, the first, second, or third tumor antigen is cKIT. In some embodiments, the first, second, or third tumor-targeting moiety comprises:
(i) a HCDR1, HCDR2, and/or HCDR3 from SEQ ID NO: 3 (or a sequence with no more than 1, 2, 3, or 4 mutations, e.g., substitutions, additions, or deletions),
(ii) a VH of SEQ ID NO: 3 (or a sequence having at least about 75%, 80%, 85%, 90%, 95%, or 99% sequence identity thereto),
(iii) a LCDR1, LCDR2, and/or LCDR3 from SEQ ID NO: 4 (or a sequence with no more than 1, 2, 3, or 4 mutations, e.g., substitutions, additions, or deletions), and/or
(iv) a VL of SEQ ID NO: 4 (or a sequence having at least about 75%, 80%, 85%, 90%, 95%, or 99% sequence identity thereto).

In some embodiments, the first, second, or third tumor antigen is FLT3. In some embodiments, the first, second, or third tumor-targeting moiety comprises:
(i) a HCDR1, HCDR2, and/or HCDR3 from SEQ ID NO: 5 (or a sequence with no more than 1, 2, 3, or 4 mutations, e.g., substitutions, additions, or deletions),
(ii) a VH of SEQ ID NO: 5 (or a sequence having at least about 75%, 80%, 85%, 90%, 95%, or 99% sequence identity thereto),
(iii) a LCDR1, LCDR2, and/or LCDR3 from SEQ ID NO: 6 (or a sequence with no more than 1, 2, 3, or 4 mutations, e.g., substitutions, additions, or deletions), and/or
(iv) a VL of SEQ ID NO: 6 (or a sequence having at least about 75%, 80%, 85%, 90%, 95%, or 99% sequence identity thereto).

In some embodiments, the first, second, or third tumor antigen is MPL. In some embodiments, the first, second, or third tumor-targeting moiety comprises:
(i) (a) a HCDR1, HCDR2, and/or HCDR3 from SEQ ID NO: 9 (or a sequence with no more than 1, 2, 3, or 4 mutations, e.g., substitutions, additions, or deletions),
(b) a VH of SEQ ID NO: 9 (or a sequence having at least about 75%, 80%, 85%, 90%, 95%, or 99% sequence identity thereto),
(c) a LCDR1, LCDR2, and/or LCDR3 from SEQ ID NO: 10 (or a sequence with no more than 1, 2, 3, or 4 mutations, e.g., substitutions, additions, or deletions), and/or
(d) a VL of SEQ ID NO: 10 (or a sequence having at least about 75%, 80%, 85%, 90%, 95%, or 99% sequence identity thereto); or
(ii) (a) a HCDR1, HCDR2, and/or HCDR3 from SEQ ID NO: 11 (or a sequence with no more than 1, 2, 3, or 4 mutations, e.g., substitutions, additions, or deletions),
(b) a VH of SEQ ID NO: 11 (or a sequence having at least about 75%, 80%, 85%, 90%, 95%, or 99% sequence identity thereto),
(c) a LCDR1, LCDR2, and/or LCDR3 from SEQ ID NO: 12 (or a sequence with no more than 1, 2, 3, or 4 mutations, e.g., substitutions, additions, or deletions), and/or
(d) a VL of SEQ ID NO: 12 (or a sequence having at least about 75%, 80%, 85%, 90%, 95%, or 99% sequence identity thereto).

In some embodiments, the first, second, or third tumor antigen is ITGB3.

In some embodiments, the first, second, or third tumor antigen is ITGB2.

In some embodiments, the first, second, or third tumor antigen is GP5.

In some embodiments, the first, second, or third tumor antigen is GP6.

In some embodiments, the first, second, or third tumor antigen is GP9.

In some embodiments, the first, second, or third tumor antigen is GP1BA.

In some embodiments, the first, second, or third tumor antigen is DSC2. In some embodiments, the first, second, or third tumor-targeting moiety comprises:
(i) (a) a HCDR1, HCDR2, and/or HCDR3 from SEQ ID NO: 15 (or a sequence with no more than 1, 2, 3, or 4 mutations, e.g., substitutions, additions, or deletions),
   (b) a VH of SEQ ID NO: 15 (or a sequence having at least about 75%, 80%, 85%, 90%, 95%, or 99% sequence identity thereto),
   (c) a LCDR1, LCDR2, and/or LCDR3 from SEQ ID NO: 16 (or a sequence with no more than 1, 2, 3, or 4 mutations, e.g., substitutions, additions, or deletions), and/or
   (d) a VL of SEQ ID NO: 16 (or a sequence having at least about 75%, 80%, 85%, 90%, 95%, or 99% sequence identity thereto); or
(ii) (a) a HCDR1, HCDR2, and/or HCDR3 from SEQ ID NO: 17 (or a sequence with no more than 1, 2, 3, or 4 mutations, e.g., substitutions, additions, or deletions),
   (b) a VH of SEQ ID NO: 17 (or a sequence having at least about 75%, 80%, 85%, 90%, 95%, or 99% sequence identity thereto),
   (c) a LCDR1, LCDR2, and/or LCDR3 from SEQ ID NO: 18 (or a sequence with no more than 1, 2, 3, or 4 mutations, e.g., substitutions, additions, or deletions), and/or
   (d) a VL of SEQ ID NO: 18 (or a sequence having at least about 75%, 80%, 85%, 90%, 95%, or 99% sequence identity thereto).

In some embodiments, the first, second, or third tumor antigen is FCGR2A. In some embodiments, the first, second, or third tumor-targeting moiety comprises:
(i) (a) a HCDR1, HCDR2, and/or HCDR3 from SEQ ID NO: 19 (or a sequence with no more than 1, 2, 3, or 4 mutations, e.g., substitutions, additions, or deletions),
   (b) a VH of SEQ ID NO: 19 (or a sequence having at least about 75%, 80%, 85%, 90%, 95%, or 99% sequence identity thereto),
   (c) a LCDR1, LCDR2, and/or LCDR3 from SEQ ID NO: 20 (or a sequence with no more than 1, 2, 3, or 4 mutations, e.g., substitutions, additions, or deletions), and/or
   (d) a VL of SEQ ID NO: 20 (or a sequence having at least about 75%, 80%, 85%, 90%, 95%, or 99% sequence identity thereto);
(ii) (a) a HCDR1, HCDR2, and/or HCDR3 from SEQ ID NO: 21 (or a sequence with no more than 1, 2, 3, or 4 mutations, e.g., substitutions, additions, or deletions),
   (b) a VH of SEQ ID NO: 21 (or a sequence having at least about 75%, 80%, 85%, 90%, 95%, or 99% sequence identity thereto),
   (c) a LCDR1, LCDR2, and/or LCDR3 from SEQ ID NO: 22 (or a sequence with no more than 1, 2, 3, or 4 mutations, e.g., substitutions, additions, or deletions), and/or
   (d) a VL of SEQ ID NO: 22 (or a sequence having at least about 75%, 80%, 85%, 90%, 95%, or 99% sequence identity thereto); or
(iii) (a) a HCDR1, HCDR2, and/or HCDR3 from SEQ ID NO: 23 (or a sequence with no more than 1, 2, 3, or 4 mutations, e.g., substitutions, additions, or deletions),
   (b) a VH of SEQ ID NO: 23 (or a sequence having at least about 75%, 80%, 85%, 90%, 95%, or 99% sequence identity thereto),
   (c) a LCDR1, LCDR2, and/or LCDR3 from SEQ ID NO: 24 (or a sequence with no more than 1, 2, 3, or 4 mutations, e.g., substitutions, additions, or deletions), and/or
   (d) a VL of SEQ ID NO: 24 (or a sequence having at least about 75%, 80%, 85%, 90%, 95%, or 99% sequence identity thereto).

In some embodiments, the first, second, or third tumor antigen is TNFRSF10A or TNFRSF10B. In some embodiments, the first, second, or third tumor-targeting moiety comprises:
(i) (a) a HCDR1, HCDR2, and/or HCDR3 from SEQ ID NO: 25 (or a sequence with no more than 1, 2, 3, or 4 mutations, e.g., substitutions, additions, or deletions),
   (b) a VH of SEQ ID NO: 25 (or a sequence having at least about 75%, 80%, 85%, 90%, 95%, or 99% sequence identity thereto),
   (c) a LCDR1, LCDR2, and/or LCDR3 from SEQ ID NO: 26 (or a sequence with no more than 1, 2, 3, or 4 mutations, e.g., substitutions, additions, or deletions), and/or
   (d) a VL of SEQ ID NO: 26 (or a sequence having at least about 75%, 80%, 85%, 90%, 95%, or 99% sequence identity thereto); or
(ii) (a) a HCDR1, HCDR2, and/or HCDR3 from SEQ ID NO: 27 (or a sequence with no more than 1, 2, 3, or 4 mutations, e.g., substitutions, additions, or deletions),
   (b) a VH of SEQ ID NO: 27 (or a sequence having at least about 75%, 80%, 85%, 90%, 95%, or 99% sequence identity thereto),
   (c) a LCDR1, LCDR2, and/or LCDR3 from SEQ ID NO: 28 (or a sequence with no more than 1, 2, 3, or 4 mutations, e.g., substitutions, additions, or deletions), and/or
   (d) a VL of SEQ ID NO: 28 (or a sequence having at least about 75%, 80%, 85%, 90%, 95%, or 99% sequence identity thereto);
(iii) (a) a HCDR1, HCDR2, and/or HCDR3 from SEQ ID NO: 29 (or a sequence with no more than 1, 2, 3, or 4 mutations, e.g., substitutions, additions, or deletions),
   (b) a VH of SEQ ID NO: 29 (or a sequence having at least about 75%, 80%, 85%, 90%, 95%, or 99% sequence identity thereto),
   (c) a LCDR1, LCDR2, and/or LCDR3 from SEQ ID NO: 30 (or a sequence with no more than 1, 2, 3, or 4 mutations, e.g., substitutions, additions, or deletions), and/or
   (d) a VL of SEQ ID NO: 30 (or a sequence having at least about 75%, 80%, 85%, 90%, 95%, or 99% sequence identity thereto);
(iv) (a) a HCDR1, HCDR2, and/or HCDR3 from SEQ ID NO: 31 (or a sequence with no more than 1, 2, 3, or 4 mutations, e.g., substitutions, additions, or deletions),
   (b) a VH of SEQ ID NO: 31 (or a sequence having at least about 75%, 80%, 85%, 90%, 95%, or 99% sequence identity thereto),
   (c) a LCDR1, LCDR2, and/or LCDR3 from SEQ ID NO: 32 (or a sequence with no more than 1, 2, 3, or 4 mutations, e.g., substitutions, additions, or deletions), and/or
   (d) a VL of SEQ ID NO: 32 (or a sequence having at least about 75%, 80%, 85%, 90%, 95%, or 99% sequence identity thereto); or
(v) (a) a HCDR1, HCDR2, and/or HCDR3 from SEQ ID NO: 33 (or a sequence with no more than 1, 2, 3, or 4 mutations, e.g., substitutions, additions, or deletions),
   (b) a VH of SEQ ID NO: 33 (or a sequence having at least about 75%, 80%, 85%, 90%, 95%, or 99% sequence identity thereto),
   (c) a LCDR1, LCDR2, and/or LCDR3 from SEQ ID NO: 34 (or a sequence with no more than 1, 2, 3, or 4 mutations, e.g., substitutions, additions, or deletions), and/or
   (d) a VL of SEQ ID NO: 34 (or a sequence having at least about 75%, 80%, 85%, 90%, 95%, or 99% sequence identity thereto).

In some embodiments, the first, second, or third tumor antigen is TM4SF1. In some embodiments, the first, second, or third tumor-targeting moiety comprises:
(i) a HCDR1, HCDR2, and/or HCDR3 from SEQ ID NO: 35 (or a sequence with no more than 1, 2, 3, or 4 mutations, e.g., substitutions, additions, or deletions),
(ii) a VH of SEQ ID NO: 35 (or a sequence having at least about 75%, 80%, 85%, 90%, 95%, or 99% sequence identity thereto),
(iii) a LCDR1, LCDR2, and/or LCDR3 from SEQ ID NO: 36 (or a sequence with no more than 1, 2, 3, or 4 mutations, e.g., substitutions, additions, or deletions), and/or
(iv) a VL of SEQ ID NO: 36 (or a sequence having at least about 75%, 80%, 85%, 90%, 95%, or 99% sequence identity thereto).

In another aspect, the disclosure provides an isolated nucleic acid molecule encoding any multispecific or multifunctional molecule described herein. In another aspect, the disclosure provides an isolated nucleic acid molecule, which comprises the nucleotide sequence encoding any of the multispecific or multifunctional molecules described herein, or a nucleotide sequence substantially homologous thereto (e.g., at least 80%, 90%, 95%, or 99.9% identical thereto). In another aspect, the disclosure provides a host cell comprising a nucleic acid molecule or a vector described herein.

In another aspect, the disclosure provides a method of making, e.g., producing, a multispecific or multifunctional molecule polypeptide described herein, comprising culturing a host cell described herein, under suitable conditions, e.g., conditions suitable for gene expression and/or homo- or heterodimerization.

In another aspect, the disclosure provides a pharmaceutical composition comprising a multispecific or multifunctional molecule polypeptide described herein and a pharmaceutically acceptable carrier, excipient, or stabilizer.

In another aspect, the disclosure provides a method of treating a cancer, comprising administering to a subject in need thereof a multispecific or multifunctional molecule polypeptide described herein, wherein the multispecific antibody is administered in an amount effective to treat the cancer. In some embodiments, the subject has tumor cells that express the first, second, or third tumor antigen, e.g., the subject has tumor cells that express a tumor antigen chosen from CD34, CD41, G6B, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1. In some embodiments, the subject has the JAK2 V617F mutation. In some embodiments, the subject has a calreticulin mutation. In some embodiments, the subject has a MPL mutation. In some embodiments, the cancer is a hematological cancer, optionally wherein the cancer is a myeloproliferative neoplasm, e.g., primary or idiopathic myelofibrosis (MF), essential thrombocytosis (ET), polycythemia vera (PV), or chronic myelogenous leukemia (CML). In some embodiments, the cancer is myelofibrosis. In some embodiments, the cancer is a solid tumor cancer. In some embodiments, the solid tumor cancer is one or more of pancreatic (e.g., pancreatic adenocarcinoma), breast, colorectal, lung (e.g., small or non-small cell lung cancer), skin, ovarian, or liver cancer.

In some embodiments, the method further comprises administering a second therapeutic treatment. In some embodiments, second therapeutic treatment comprises a therapeutic agent (e.g., a chemotherapeutic agent, a biologic agent, hormonal therapy), radiation, or surgery. In some embodiments, therapeutic agent is selected from: a chemotherapeutic agent, or a biologic agent.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIGS. 1A-1C are schematic representations of exemplary formats and configurations of functional moieties attached to a dimerization module, e.g., an immunoglobulin constant domain. FIG. 1A depicts moieties A, B, C and D, covalently linked to a heterodimeric Fc domain. FIG. 1B depicts moieties A, B, C and D, covalently linked to a homodimeric Fc domain. FIG. 1C depicts moieties A, B, C and D, covalently linked to heterodimeric heavy and light constant domains (e.g., a Fab CH₁ and a Fab CL). In some embodiments, the functional moiety is a first tumor-targeting moiety that binds to a first tumor antigen. In some embodiments, the functional moiety is a second tumor-targeting moiety that binds to a second tumor antigen. In some embodiments, the functional moiety is a third tumor-targeting moiety that binds to a second tumor antigen. In some embodiments, the first, second, and optionally the third tumor antigens are each independently chosen from: CD34, CD41, G6B, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1. In some embodiments, the functional moiety is an immune cell engager chosen from a T cell engager, an NK cell engager, a B cell engager, a dendritic cell engager, or a macrophage cell engager. In some embodiments, the functional moiety is a cytokine molecule. In some embodiments, the functional moiety is a modulator of a cytokine molecule. In some embodiments, the functional moiety is a stromal modifying moiety.

FIGS. 2A-2D are schematics showing exemplary multispecific molecules comprising a TGFβ inhibitor. In some embodiments, the TGFβ inhibitor comprises a TGF-beta receptor ECD homodimer. In some embodiments, the TGFβ inhibitor comprises a TGFBR2 ECD heterodimer. In FIGS. 2A and 2B, the two TGFBR ECD domains are linked to the C-terminus of two Fc regions. In some embodiments, the CH1-Fc-TGFBR ECD region shown in FIG. 2A or 2B comprises the amino acid sequence of SEQ ID NO: 192 or 193. In some embodiments, the Fc-TGFBR ECD region shown in FIG. 2A or 2B comprises the amino acid sequence of SEQ ID NO: 194 or 195. In FIGS. 2C and 2D, the two TGFBR ECD domains are linked to CH1 and CL, respectively. In some embodiments, the TGFBR ECD-CH1-Fc region shown in FIG. 2C or 2D comprises the amino acid sequence of SEQ ID NO: 196 or 197. In some embodiments, the TGFBR ECD-CL region shown in FIG. 2C or 2D comprises the amino acid sequence of SEQ ID NO: 198 or 199. In some embodiments, the multispecific molecule comprises a binding moiety A and a binding moiety B. In some embodiments, the binding moiety A or binding moiety B is a tumor-targeting moiety disclosed herein.

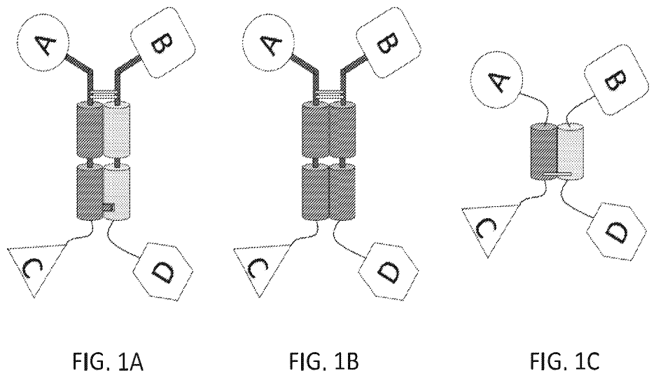

### DETAILED DESCRIPTION OF THE INVENTION

Disclosed herein are multifunctional molecules (also referred to herein as “multispecific molecules”) that include a plurality of (e.g., two or more) functionalities (or binding specificities), comprising (i) a first tumor-targeting moiety that binds to a first tumor antigen and (ii) a second tumor-targeting moiety that binds to a second tumor antigen, wherein the first and second tumor antigens are each independently chosen from: CD34, CD41, G6B, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1. In some embodiments, the first tumor antigen is different from the second tumor antigen. In some embodiments, the multifunctional molecule further comprises one, two, or all of: (iii) an immune cell engager chosen from a T cell engager, an NK cell engager, a B cell engager, a dendritic cell engager, or a macrophage cell engager; (iv) a cytokine molecule or a modulator of a cytokine molecule; and (v) a stromal modifying moiety. In some embodiments, the multifunctional molecule further comprises (vi) a third tumor-targeting moiety that binds to a third tumor antigen. In some embodiments, the third tumor antigen is chosen from: CD34, CD41, G6B, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1. In some embodiments, the third tumor antigen is different from the first or second tumor antigen. In some embodiments, the first, the second, and optionally the third tumor antigens are expressed on the same tumor cell.

In an embodiment, the multispecific or multifunctional molecule is a bispecific (or bifunctional) molecule, a trispecific (or trifunctional) molecule, or a tetraspecific (or tetrafunctional) molecule.

Without being bound by theory, the multispecific or multifunctional molecules disclosed herein are expected to localize (e.g., bridge) and/or activate an immune cell (e.g., an immune effector cell chosen from a T cell, an NK cell, a B cell, a dendritic cell or a macrophage), in the presence of a cell expressing the first, the second, and optionally the third tumor antigens. Increasing the proximity and/or activity of the immune cell, in the presence of the cell expressing the first, the second, and optionally the third tumor antigens, using the multispecific or multifunctional molecules described herein is expected to enhance an immune response against the target cell, thereby providing a more effective therapy.

Novel multifunctional, e.g., multispecific, molecules that include (i) a stromal modifying moiety and (ii) a first tumor-targeting moiety that binds to a first tumor antigen, a second tumor-targeting moiety that binds to a second tumor antigen, and optionally a third tumor-targeting moiety that binds to a third tumor antigen. Without being bound by theory, the multifunctional molecules disclosed herein are believed to inter alia target (e.g., localize to) a cancer site, and alter the tumor stroma, e.g., alter the tumor microenvironment near the cancer site. The multifunctional molecules can further include one or both of: an immune cell engager (e.g., chosen from one, two, three, or all of a T cell engager, NK cell engager, a B cell engager, a dendritic cell engager, or a macrophage cell engager); and/or a cytokine molecule or a modulator of a cytokine molecule. Accordingly, provided herein are, inter alia, multifunctional, e.g., multispecific molecules, that include the aforesaid moieties, nucleic acids encoding the same, methods of producing the aforesaid molecules, and methods of treating a cancer using the aforesaid molecules.

Accordingly, provided herein are, inter alia, multispecific or multifunctional molecules (e.g., multispecific or multifunctional antibody molecules) that include the aforesaid moieties, nucleic acids encoding the same, methods of producing the aforesaid molecules, and methods of treating a disease or disorder, e.g., cancer, using the aforesaid molecules.

### Definitions

In some embodiments, the multifunctional molecule includes an immune cell engager. “An immune cell engager” refers to one or more binding specificities that bind and/or activate an immune cell, e.g., a cell involved in an immune response. In embodiments, the immune cell is chosen from a T cell, an NK cell, a B cell, a dendritic cell, and/or the macrophage cell. The immune cell engager can be an antibody molecule, a receptor molecule (e.g., a full length receptor, receptor fragment, or fusion thereof (e.g., a receptor-Fc fusion)), or a ligand molecule (e.g., a full length ligand, ligand fragment, or fusion thereof (e.g., a ligand-Fc fusion)) that binds to the immune cell antigen (e.g., the T cell, the NK cell antigen, the B cell antigen, the dendritic cell antigen, and/or the macrophage cell antigen). In embodiments, the immune cell engager specifically binds to the target immune cell, e.g., binds preferentially to the target immune cell. For example, when the immune cell engager is an antibody molecule, it binds to an immune cell antigen (e.g., a T cell antigen, an NK cell antigen, a B cell antigen, a dendritic cell antigen, and/or a macrophage cell antigen) with a dissociation constant of less than about 10 nM.

In some embodiments, the multifunctional molecule includes a cytokine molecule. As used herein, a “cytokine molecule” refers to full length, a fragment or a variant of a cytokine; a cytokine further comprising a receptor domain, e.g., a cytokine receptor dimerizing domain; or an agonist of a cytokine receptor, e.g., an antibody molecule (e.g., an agonistic antibody) to a cytokine receptor, that elicits at least one activity of a naturally-occurring cytokine. In some embodiments the cytokine molecule is chosen from interleukin-2 (IL-2), interleukin-7 (IL-7), interleukin-12 (IL-12), interleukin-15 (IL-15), interleukin-18 (IL-18), interleukin-21 (IL-21), or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain. In other embodiments, the cytokine molecule is an agonist of a cytokine receptor, e.g., an antibody molecule (e.g., an agonistic antibody) to a cytokine receptor chosen from an IL-15Ra or IL-21R.

As used herein, the term “molecule” as used in, e.g., antibody molecule, cytokine molecule, receptor molecule, includes full-length, naturally-occurring molecules, as well as variants, e.g., functional variants (e.g., truncations, fragments, mutated (e.g., substantially similar sequences) or derivatized form thereof), so long as at least one function and/or activity of the unmodified (e.g., naturally-occurring) molecule remains.

In some embodiments, the multifunctional molecule includes a stromal modifying moiety. A “stromal modifying moiety,” as used herein refers to an agent, e.g., a protein (e.g., an enzyme), that is capable of altering, e.g., degrading a component of, the stroma. In embodiments, the component of the stroma is chosen from, e.g., an ECM component, e.g., a glycosaminoglycan, e.g., hyaluronan (also known as hyaluronic acid or HA), chondroitin sulfate, chondroitin, dermatan sulfate, heparin sulfate, heparin, entactin, tenascin, aggrecan and keratin sulfate; or an extracellular protein, e.g., collagen, laminin, elastin, fibrinogen, fibronectin, and vitronectin.

Certain terms are defined below.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values.

“Antibody molecule” as used herein refers to a protein, e.g., an immunoglobulin chain or fragment thereof, comprising at least one immunoglobulin variable domain sequence. An antibody molecule encompasses antibodies (e.g., full-length antibodies) and antibody fragments. In an embodiment, an antibody molecule comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)₂ fragments, and single chain variable fragments (scFvs).

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

In embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule. “Bispecific antibody molecule” as used herein refers to an antibody molecule that has specificity for more than one (e.g., two, three, four, or more) epitope and/or antigen.

“Antigen” (Ag) as used herein refers to a molecule that can provoke an immune response, e.g., involving activation of certain immune cells and/or antibody generation. Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full length nucleotide sequence of a gene, nor does an antigen need to be encoded by a gene at all. In embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response.

The “antigen-binding site,” or “binding portion” of an antibody molecule refers to the part of an antibody molecule, e.g., an immunoglobulin (Ig) molecule, that participates in antigen binding. In embodiments, the antigen binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions,” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4.

“Cancer” as used herein can encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors.

As used herein, an “immune cell” refers to any of various cells that function in the immune system, e.g., to protect against agents of infection and foreign matter. In embodiments, this term includes leukocytes, e.g., neutrophils, eosinophils, basophils, lymphocytes, and monocytes. Innate leukocytes include phagocytes (e.g., macrophages, neutrophils, and dendritic cells), mast cells, eosinophils, basophils, and natural killer cells. Innate leukocytes identify and eliminate pathogens, either by attacking larger pathogens through contact or by engulfing and then killing microorganisms, and are mediators in the activation of an adaptive immune response. The cells of the adaptive immune system are special types of leukocytes, called lymphocytes. B cells and T cells are important types of lymphocytes and are derived from hematopoietic stem cells in the bone marrow. B cells are involved in the humoral immune response, whereas T cells are involved in cell-mediated immune response. The term “immune cell” includes immune effector cells.

“Immune effector cell,” as that term is used herein, refers to a cell that is involved in an immune response, e.g., in the promotion of an immune effector response. Examples of immune effector cells include, but are not limited to, T cells, e.g., alpha/beta T cells and gamma/delta T cells, B cells, natural killer (NK) cells, natural killer T (NK T) cells, and mast cells.

The term “effector function” or “effector response” refers to a specialized function of a cell. Effector function of a T cell, for example, may be cytolytic activity or helper activity including the secretion of cytokines.

The compositions and methods of the present invention encompass polypeptides and nucleic acids having the sequences specified, or sequences substantially identical or similar thereto, e.g., sequences at least 80%, 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 80%, 85%, 90%, 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 80%, 85%, 90%, 91%, 92%, 93%, 94%, 95%, 96%, 97%, 98% or 99% identity to a reference sequence, e.g., a sequence provided herein.

The term “variant” refers to a polypeptide that has a substantially identical amino acid sequence to a reference amino acid sequence, or is encoded by a substantially identical nucleotide sequence. In some embodiments, the variant is a functional variant.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecule of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, the term “transforming growth factor beta-1 (TGF-beta 1)” refers to a protein that in humans is encoded by the gene TGFB1, or its orthologs. Swiss-Prot accession number P01137 provides exemplary human TGF-beta 1 amino acid sequences. An exemplary immature human TGF-beta 1 amino acid sequence is provided in SEQ ID NO: 200. An exemplary mature human TGF-beta 1 amino acid sequence is provided in SEQ ID NO: 117.

As used herein, the term “transforming growth factor beta-2 (TGF-beta 2)” refers to a protein that in humans is encoded by the gene TGFB2, or its orthologs. Swiss-Prot accession number P61812 provides exemplary human TGF-beta 2 amino acid sequences. An exemplary immature human TGF-beta 2 amino acid sequence is provided in SEQ ID NO: 93. An exemplary mature human TGF-beta 2 amino acid sequence is provided in SEQ ID NO: 118.

As used herein, the term “transforming growth factor beta-3 (TGF-beta 3)” refers to a protein that in humans is encoded by the gene TGFB3, or its orthologs. Swiss-Prot accession number P10600 provides exemplary human TGF-beta 3 amino acid sequences. An exemplary immature human TGF-beta 3 amino acid sequence is provided in SEQ ID NO: 94. An exemplary mature human TGF-beta 3 amino acid sequence is provided in SEQ ID NO: 119.

As used herein, a “TGF-beta receptor polypeptide” refers to a TGF-beta receptor (e.g., TGFBR1, TGFBR2, or TGFBR3) or its fragment, or variant thereof.

As used herein, the term “transforming growth factor beta receptor type 1 (TGFBR1)” (also known as ALK-5 or SKR4) refers to a protein that in humans is encoded by the gene TGFBR1, or its orthologs. Swiss-Prot accession number P36897 provides exemplary human TGFBR1 amino acid sequences. Exemplary immature human TGFBR1 amino acid sequences are provided in SEQ ID NOs: 95, 96, and 97. Exemplary mature human TGFBR1 amino acid sequences are provided in SEQ ID NOs: 120, 121, and 122. As used herein, a “TGFBR1 polypeptide” refers to a TGFBR1 or its fragment, or variant thereof.

As used herein, the term “transforming growth factor beta receptor type 2 (TGFBR2)” refers to a protein that in humans is encoded by the gene TGFBR2, or its orthologs. Swiss-Prot accession number P37173 provides exemplary human TGFBR2 amino acid sequences. Exemplary immature human TGFBR2 amino acid sequences are provided in SEQ ID NOs: 98 and 99. Exemplary mature human TGFBR2 amino acid sequences are provided in SEQ ID NOs: 123 and 124. As used herein, a “TGFBR2 polypeptide” refers to a TGFBR2 or its fragment, or variant thereof.

As used herein, the term “transforming growth factor beta receptor type 3 (TGFBR3)” refers to a protein that in humans is encoded by the gene TGFBR3, or its orthologs. Swiss-Prot accession number Q03167 provides exemplary human TGFBR3 amino acid sequences. Exemplary immature human TGFBR3 amino acid sequences are provided in SEQ ID NOs: 106 and 107. Exemplary mature human TGFBR3 amino acid sequences are provided in SEQ ID NOs: 125 and 126. As used herein, a “TGFBR3 polypeptide” refers to a TGFBR3 or its fragment, or variant thereof.

Various aspects of the invention are described in further detail below. Additional definitions are set out throughout the specification.

### Antibody Molecules

In embodiments, the antibody molecule binds to a cancer antigen, e.g., a tumor antigen or a stromal antigen. In some embodiments, the cancer antigen is, e.g., a mammalian, e.g., a human, cancer antigen. In other embodiments, the antibody molecule binds to an immune cell antigen, e.g., a mammalian, e.g., a human, immune cell antigen. For example, the antibody molecule binds specifically to an epitope, e.g., linear or conformational epitope, on the cancer antigen or the immune cell antigen.

In an embodiment, an antibody molecule is a monospecific antibody molecule and binds a single epitope. E.g., a monospecific antibody molecule having a plurality of immunoglobulin variable domain sequences, each of which binds the same epitope.

In an embodiment an antibody molecule is a multispecific or multifunctional antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a multispecific antibody molecule comprises a third, fourth or fifth immunoglobulin variable domain. In an embodiment, a multispecific antibody molecule is a bispecific antibody molecule, a trispecific antibody molecule, or a tetraspecific antibody molecule.

In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv or a Fab, or fragment thereof, have binding specificity for a first epitope and a scFv or a Fab, or fragment thereof, have binding specificity for a second epitope.

In an embodiment, an antibody molecule comprises a diabody, and a single-chain molecule, as well as an antigen-binding fragment of an antibody (e.g., Fab, F(ab′)2, and Fv). For example, an antibody molecule can include a heavy (H) chain variable domain sequence (abbreviated herein as VH), and a light (L) chain variable domain sequence (abbreviated herein as VL). In an embodiment an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The a preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

Antibody molecules include intact molecules as well as functional fragments thereof. Constant regions of the antibody molecules can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3).

Each VH and VL typically includes three CDRs and four FRs, arranged from amino-terminus to carboxy-terminus in the following order: FR1, CDR1, FR2, CDR2, FR3, CDR3, FR4.

The antibody molecule can be a polyclonal or a monoclonal antibody.

The terms “monoclonal antibody” or “monoclonal antibody composition” as used herein refer to a preparation of antibody molecules of single molecular composition. A monoclonal antibody composition displays a single binding specificity and affinity for a particular epitope.

A monoclonal antibody can be made by hybridoma technology or by methods that do not use hybridoma technology (e.g., recombinant methods).

The antibody can be recombinantly produced, e.g., produced by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al.

International Publication No. WO 92/01047; Garrard et al. International Publication No. WO 92/09690; Ladner et al. International Publication No. WO 90/02809; Fuchs et al. (1991) Bio Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody molecule can be one in which the variable region, or a portion thereof, e.g., the CDRs, are generated in a non-human organism, e.g., a rat or mouse. Chimeric, CDR-grafted, and humanized antibodies are within the invention. Antibody molecules generated in a non-human organism, e.g., a rat or mouse, and then modified, e.g., in the variable framework or constant region, to decrease antigenicity in a human are within the invention.

An “effectively human” protein is a protein that does substantially not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding to the antigen. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody molecule can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibody molecules can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibody molecules in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

### Multispecific or Multifunctional Antibody Molecules

Exemplary structures of multispecific and multifunctional molecules defined herein are described throughout. Exemplary structures are further described in: Weidle U et al. (2013) The Intriguing Options of Multispecific Antibody Formats for Treatment of Cancer. Cancer Genomics & Proteomics 10: 1-18 (2013); and Spiess C et al. (2015) Alternative molecular formats and therapeutic applications for bispecific antibodies. Molecular Immunology 67: 95-106; the full contents of each of which is incorporated by reference herein).

In embodiments, multispecific antibody molecules can comprise more than one antigen-binding site, where different sites are specific for different antigens. In embodiments, multispecific antibody molecules can bind more than one (e.g., two or more) epitopes on the same antigen. In embodiments, multispecific antibody molecules comprise an antigen-binding site specific for a target cell (e.g., cancer cell) and a different antigen-binding site specific for an immune effector cell. In one embodiment, the multispecific antibody molecule is a bispecific antibody molecule. Bispecific antibody molecules can be classified into five different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates.

BsIgG is a format that is monovalent for each antigen. Exemplary BsIgG formats include but are not limited to crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, κλ-body, orthogonal Fab. See Spiess et al. Mol. Immunol. 67(2015):95-106. Exemplary BsIgGs include catumaxomab (Fresenius Biotech, Trion Pharma, Neopharm), which contains an anti-CD3 arm and an anti-EpCAM arm; and ertumaxomab (Neovii Biotech, Fresenius Biotech), which targets CD3 and HER2. In some embodiments, BsIgG comprises heavy chains that are engineered for heterodimerization. For example, heavy chains can be engineered for heterodimerization using a “knobs-into-holes” strategy, a SEED platform, a common heavy chain (e.g., in κλ-bodies), and use of heterodimeric Fc regions. See Spiess et al. Mol. Immunol. 67(2015):95-106. Strategies that have been used to avoid heavy chain pairing of homodimers in BsIgG include knobs-in-holes, duobody, azymetric, charge pair, HA-TF, SEEDbody, and differential protein A affinity. See Id. BsIgG can be produced by separate expression of the component antibodies in different host cells and subsequent purification/assembly into a BsIgG. BsIgG can also be produced by expression of the component antibodies in a single host cell. BsIgG can be purified using affinity chromatography, e.g., using protein A and sequential pH elution.

IgG appended with an additional antigen-binding moiety is another format of bispecific antibody molecules. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). See Id. Examples of appended IgG formats include dual variable domain IgG (DVD-Ig), IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015):95-106. An example of an IgG-scFv is MM-141 (Merrimack Pharmaceuticals), which binds IGF-1R and HER3. Examples of DVD-Ig include ABT-981 (AbbVie), which binds IL-1α and IL-1β; and ABT-122 (AbbVie), which binds TNF and IL-17A.

Bispecific antibody fragments (BsAb) are a format of bispecific antibody molecules that lack some or all of the antibody constant domains. For example, some BsAb lack an Fc region. In embodiments, bispecific antibody fragments include heavy and light chain regions that are connected by a peptide linker that permits efficient expression of the BsAb in a single host cell. Exemplary bispecific antibody fragments include but are not limited to nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, tandem scFv-Fc, and intrabody. See Id. For example, the BiTE format comprises tandem scFvs, where the component scFvs bind to CD3 on T cells and a surface antigen on cancer cells

Bispecific fusion proteins include antibody fragments linked to other proteins, e.g., to add additional specificity and/or functionality. An example of a bispecific fusion protein is an immTAC, which comprises an anti-CD3 scFv linked to an affinity-matured T-cell receptor that recognizes HLA-presented peptides. In embodiments, the dock-and-lock (DNL) method can be used to generate bispecific antibody molecules with higher valency. Also, fusions to albumin binding proteins or human serum albumin can be extend the serum half-life of antibody fragments. See Id.

In embodiments, chemical conjugation, e.g., chemical conjugation of antibodies and/or antibody fragments, can be used to create BsAb molecules. See Id. An exemplary bispecific antibody conjugate includes the CovX-body format, in which a low molecular weight drug is conjugated site-specifically to a single reactive lysine in each Fab arm or an antibody or fragment thereof. In embodiments, the conjugation improves the serum half-life of the low molecular weight drug. An exemplary CovX-body is CVX-241 (NCT01004822), which comprises an antibody conjugated to two short peptides inhibiting either VEGF or Ang2. See Id.

The antibody molecules can be produced by recombinant expression, e.g., of at least one or more component, in a host system. Exemplary host systems include eukaryotic cells (e.g., mammalian cells, e.g., CHO cells, or insect cells, e.g., SF9 or S2 cells) and prokaryotic cells (e.g., E. coli). Bispecific antibody molecules can be produced by separate expression of the components in different host cells and subsequent purification/assembly. Alternatively, the antibody molecules can be produced by expression of the components in a single host cell. Purification of bispecific antibody molecules can be performed by various methods such as affinity chromatography, e.g., using protein A and sequential pH elution. In other embodiments, affinity tags can be used for purification, e.g., histidine-containing tag, myc tag, or streptavidin tag.

### CDR-Grafted Scaffolds

In embodiments, the antibody molecule is a CDR-grafted scaffold domain. In embodiments, the scaffold domain is based on a fibronectin domain, e.g., fibronectin type III domain. The overall fold of the fibronectin type III (Fn3) domain is closely related to that of the smallest functional antibody fragment, the variable domain of the antibody heavy chain. There are three loops at the end of Fn3; the positions of BC, DE and FG loops approximately correspond to those of CDR1, 2 and 3 of the VH domain of an antibody. Fn3 does not have disulfide bonds; and therefore Fn3 is stable under reducing conditions, unlike antibodies and their fragments (see, e.g., WO 98/56915; WO 01/64942; WO 00/34784). An Fn3 domain can be modified (e.g., using CDRs or hypervariable loops described herein) or varied, e.g., to select domains that bind to an antigen/marker/cell described herein.

In embodiments, a scaffold domain, e.g., a folded domain, is based on an antibody, e.g., a “minibody” scaffold created by deleting three beta strands from a heavy chain variable domain of a monoclonal antibody (see, e.g., Tramontano et al., 1994, J Mol. Recognit. 7:9; and Martin et al., 1994, EMBO J. 13:5303-5309). The “minibody” can be used to present two hypervariable loops. In embodiments, the scaffold domain is a V-like domain (see, e.g., Coia et al. WO 99/45110) or a domain derived from tendamistatin, which is a 74 residue, six-strand beta sheet sandwich held together by two disulfide bonds (see, e.g., McConnell and Hoess, 1995, J Mol. Biol. 250:460). For example, the loops of tendamistatin can be modified (e.g., using CDRs or hypervariable loops) or varied, e.g., to select domains that bind to a marker/antigen/cell described herein. Another exemplary scaffold domain is a beta-sandwich structure derived from the extracellular domain of CTLA-4 (see, e.g., WO 00/60070).

Other exemplary scaffold domains include but are not limited to T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains). See, e.g., US 20040009530 and U.S. Pat. No. 7,501,121, incorporated herein by reference.

In embodiments, a scaffold domain is evaluated and chosen, e.g., by one or more of the following criteria: (1) amino acid sequence, (2) sequences of several homologous domains, (3) 3-dimensional structure, and/or (4) stability data over a range of pH, temperature, salinity, organic solvent, oxidant concentration. In embodiments, the scaffold domain is a small, stable protein domain, e.g., a protein of less than 100, 70, 50, 40 or 30 amino acids. The domain may include one or more disulfide bonds or may chelate a metal, e.g., zinc.

### Antibody-Based Fusions

A variety of formats can be generated which contain additional binding entities attached to the N or C terminus of antibodies. These fusions with single chain or disulfide stabilized Fvs or Fabs result in the generation of tetravalent molecules with bivalent binding specificity for each antigen. Combinations of scFvs and scFabs with IgGs enable the production of molecules which can recognize three or more different antigens.

### Antibody-Fab Fusion

Antibody-Fab fusions are bispecific antibodies comprising a traditional antibody to a first target and a Fab to a second target fused to the C terminus of the antibody heavy chain. Commonly the antibody and the Fab will have a common light chain. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Antibody-scFv Fusion

Antibody-scFv Fusions are bispecific antibodies comprising a traditional antibody and a scFv of unique specificity fused to the C terminus of the antibody heavy chain. The scFv can be fused to the C terminus through the Heavy Chain of the scFv either directly or through a linker peptide. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Variable Domain Immunoglobulin DVD

A related format is the dual variable domain immunoglobulin (DVD), which are composed of VH and VL domains of a second specificity place upon the N termini of the V domains by shorter linker sequences.

Other exemplary multispecific antibody formats include, e.g., those described in the following US20160114057A1, US20130243775A1, US20140051833, US20130022601, US20150017187A1, US20120201746A1, US20150133638A1, US20130266568A1, US20160145340A1, WO2015127158A1, US20150203591A1, US20140322221A1, US20130303396A1, US20110293613, US20130017200A1, US20160102135A1, WO2015197598A2, WO2015197582A1, U.S. Pat. No. 9,359,437, US20150018529, WO2016115274A1, WO2016087416A1, US20080069820A1, U.S. Pat. Nos. 9,145,588B, 7,919,257, and US20150232560A1. Exemplary multispecific molecules utilizing a full antibody-Fab/scFab format include those described in the following, U.S. Pat. No. 9,382,323B2, US20140072581A1, US20140308285A1, US20130165638A1, US20130267686A1, US20140377269A1, U.S. Pat. No. 7,741,446B2, and WO1995009917A1. Exemplary multispecific molecules utilizing a domain exchange format include those described in the following, US20150315296A1, WO2016087650A1, US20160075785A1, WO2016016299A1, US20160130347A1, US20150166670, U.S. Pat. No. 8,703,132B2, US20100316645, U.S. Pat. No. 8,227,577B2, US20130078249.

### Fc-Containing Entities (Mini-Antibodies)

Fc-containing entities, also known as mini-antibodies, can be generated by fusing scFv to the C-termini of constant heavy region domain 3 (CH3-scFv) and/or to the hinge region (scFv-hinge-Fc) of an antibody with a different specificity. Trivalent entities can also be made which have disulfide stabilized variable domains (without peptide linker) fused to the C-terminus of CH3 domains of IgGs.

### Fc-Containing Multispecific Molecules

In some embodiments, the multispecific molecules disclosed herein includes an immunoglobulin constant region (e.g., an Fc region). Exemplary Fc regions can be chosen from the heavy chain constant regions of IgG1, IgG2, IgG3 or IgG4; more particularly, the heavy chain constant region of human IgG1, IgG2, IgG3, or IgG4.

In some embodiments, the immunoglobulin chain constant region (e.g., the Fc region) is altered, e.g., mutated, to increase or decrease one or more of: Fc receptor binding, antibody glycosylation, the number of cysteine residues, effector cell function, or complement function.

In other embodiments, an interface of a first and second immunoglobulin chain constant regions (e.g., a first and a second Fc region) is altered, e.g., mutated, to increase or decrease dimerization, e.g., relative to a non-engineered interface, e.g., a naturally-occurring interface. For example, dimerization of the immunoglobulin chain constant region (e.g., the Fc region) can be enhanced by providing an Fc interface of a first and a second Fc region with one or more of: a paired protuberance-cavity (“knob-in-a hole”), an electrostatic interaction, or a strand-exchange, such that a greater ratio of heteromultimer to homomultimer forms, e.g., relative to a non-engineered interface.

In some embodiments, the multispecific molecules include a paired amino acid substitution at a position chosen from one or more of 347, 349, 350, 351, 366, 368, 370, 392, 394, 395, 397, 398, 399, 405, 407, or 409, e.g., of the Fc region of human IgG1 For example, the immunoglobulin chain constant region (e.g., Fc region) can include a paired an amino acid substitution chosen from: T366S, L368A, or Y407V (e.g., corresponding to a cavity or hole), and T366W (e.g., corresponding to a protuberance or knob).

In other embodiments, the multifunctional molecule includes a half-life extender, e.g., a human serum albumin or an antibody molecule to human serum albumin.

### Heterodimerized Antibody Molecules & Methods of Making

Various methods of producing multispecific antibodies have been disclosed to address the problem of incorrect heavy chain pairing. Exemplary methods are described below. Exemplary multispecific antibody formats and methods of making said multispecific antibodies are also disclosed in e.g., Speiss et al. Molecular Immunology 67 (2015) 95-106; and Klein et al mAbs 4:6, 653-663; November/December 2012; the entire contents of each of which are incorporated by reference herein.

Heterodimerized bispecific antibodies are based on the natural IgG structure, wherein the two binding arms recognize different antigens. IgG derived formats that enable defined monovalent (and simultaneous) antigen binding are generated by forced heavy chain heterodimerization, combined with technologies that minimize light chain mispairing (e.g., common light chain). Forced heavy chain heterodimerization can be obtained using, e.g., knob-in-hole OR strand exchange engineered domains (SEED).

### Knob-In-Hole

Knob-in-Hole as described in U.S. Pat. Nos. 5,731,116, 7,476,724 and Ridgway, J. et al. (1996) Prot. Engineering 9(7): 617-621, broadly involves: (1) mutating the CH3 domain of one or both antibodies to promote heterodimerization; and (2) combining the mutated antibodies under conditions that promote heterodimerization. “Knobs” or “protuberances” are typically created by replacing a small amino acid in a parental antibody with a larger amino acid (e.g., T366Y or T366W); “Holes” or “cavities” are created by replacing a larger residue in a parental antibody with a smaller amino acid (e.g., Y407T, T366S, L368A and/or Y407V).

For bispecific antibodies including an Fc domain, introduction of specific mutations into the constant region of the heavy chains to promote the correct heterodimerization of the Fc portion can be utilized. Several such techniques are reviewed in Klein et al. (mAbs (2012) 4:6, 1-11), the contents of which are incorporated herein by reference in their entirety. These techniques include the “knobs-into-holes” (KiH) approach which involves the introduction of a bulky residue into one of the CH3 domains of one of the antibody heavy chains. This bulky residue fits into a complementary “hole” in the other CH3 domain of the paired heavy chain so as to promote correct pairing of heavy chains (see e.g., U.S. Pat. No. 7,642,228).

Exemplary KiH mutations include S354C, T366W in the “knob” heavy chain and Y349C, T366S, L368A, Y407V in the “hole” heavy chain. Other exemplary KiH mutations are provided in Table 1, with additional optional stabilizing Fc cysteine mutations.

TABLE 1
Exemplary Fc KiH mutations and optional Cysteine mutations

| Position | Knob Mutation | Hole Mutation |
|---|---|---|
| T366 | T366W | T366S |
| L368 | — | L368A |
| Y407 | — | Y407V |

Additional Cysteine Mutations to form a stabilizing disulfide bridge

| Position | Knob CH3 | Hole CH3 |
|---|---|---|
| S354 | S354C | — |
| Y349 | — | Y349C |

Other Fc mutations are provided by Igawa and Tsunoda who identified 3 negatively charged residues in the CH3 domain of one chain that pair with three positively charged residues in the CH3 domain of the other chain. These specific charged residue pairs are: E356-K439, E357-K370, D399-K409 and vice versa. By introducing at least two of the following three mutations in chain A: E356K, E357K and D399K, as well as K370E, K409D, K439E in chain B, alone or in combination with newly identified disulfide bridges, they were able to favor very efficient heterodimerization while suppressing homodimerization at the same time (Martens T et al. A novel one-armed antic-Met antibody inhibits glioblastoma growth in vivo. Clin Cancer Res 2006; 12:6144-52; PMID:17062691). Xencor defined 41 variant pairs based on combining structural calculations and sequence information that were subsequently screened for maximal heterodimerization, defining the combination of S364H, F405A (HA) on chain A and Y349T, T394F on chain B (TF) (Moore G L et al. A novel bispecific antibody format enables simultaneous bivalent and monovalent co-engagement of distinct target antigens. MAbs 2011; 3:546-57; PMID: 22123055).

Other exemplary Fc mutations to promote heterodimerization of multispecific antibodies include those described in the following references, the contents of each of which is incorporated by reference herein, WO2016071377A1, US20140079689A1, US20160194389A1, US20160257763, WO2016071376A2, WO2015107026A1, WO2015107025A1, WO2015107015A1, US20150353636A1, US20140199294A1, U.S. Pat. No. 7,750,128B2, US20160229915A1, US20150344570A1, U.S. Pat. No. 8,003,774A1, US20150337049A1, US20150175707A1, US20140242075A1, US20130195849A1, US20120149876A1, US20140200331A1, U.S. Pat. No. 9,309,311B2, U.S. Pat. No. 8,586,713, US20140037621A1, US20130178605A1, US20140363426A1, US20140051835A1 and US20110054151A1.

Stabilizing cysteine mutations have also been used in combination with KiH and other Fc heterodimerization promoting variants, see e.g., U.S. Pat. No. 7,183,076. Other exemplary cysteine modifications include, e.g., those disclosed in US20140348839A1, U.S. Pat. No. 7,855,275B2, and U.S. Pat. No. 9,000,130B2.

### Strand Exchange Engineered Domains (SEED)

Heterodimeric Fc platform that support the design of bispecific and asymmetric fusion proteins by devising strand-exchange engineered domain (SEED) C(H)3 heterodimers are known. These derivatives of human IgG and IgA C(H)3 domains create complementary human SEED C(H)3 heterodimers that are composed of alternating segments of human IgA and IgG C(H)3 sequences. The resulting pair of SEED C(H)3 domains preferentially associates to form heterodimers when expressed in mammalian cells. SEEDbody (Sb) fusion proteins consist of [IgG1 hinge]-C(H)2-[SEED C(H)3], that may be genetically linked to one or more fusion partners (see e.g., Davis J H et al. SEEDbodies: fusion proteins based on strand exchange engineered domain (SEED) CH3 heterodimers in an Fc analogue platform for asymmetric binders or immunofusions and bispecific antibodies. Protein Eng Des Sel 2010; 23:195-202; PMID:20299542 and U.S. Pat. No. 8,871,912. The contents of each of which are incorporated by reference herein).

### Duobody

“Duobody” technology to produce bispecific antibodies with correct heavy chain pairing are known. The DuoBody technology involves three basic steps to generate stable bispecific human IgG1 antibodies in a post-production exchange reaction. In a first step, two IgG1s, each containing single matched mutations in the third constant (CH3) domain, are produced separately using standard mammalian recombinant cell lines. Subsequently, these IgG1 antibodies are purified according to standard processes for recovery and purification. After production and purification (post-production), the two antibodies are recombined under tailored laboratory conditions resulting in a bispecific antibody product with a very high yield (typically >95%) (see e.g., Labrijn et al, PNAS 2013; 110(13):5145-5150 and Labrijn et al. Nature Protocols 2014; 9(10):2450-63, the contents of each of which are incorporated by reference herein).

### Electrostatic Interactions

Methods of making multispecific antibodies using CH3 amino acid changes with charged amino acids such that homodimer formation is electrostatically unfavorable are disclosed. EP1870459 and WO 2009089004 describe other strategies for favoring heterodimer formation upon co-expression of different antibody domains in a host cell. In these methods, one or more residues that make up the heavy chain constant domain 3 (CH3), CH3-CH3 interfaces in both CH3 domains are replaced with a charged amino acid such that homodimer formation is electrostatically unfavorable and heterodimerization is electrostatically favorable. Additional methods of making multispecific molecules using electrostatic interactions are described in the following references, the contents of each of which is incorporated by reference herein, include US20100015133, U.S. Pat. No. 8,592,562B2, U.S. Pat. No. 9,200,060B2, US20140154254A1, and U.S. Pat. No. 9,358,286A1.

### Common Light Chain

Light chain mispairing needs to be avoided to generate homogenous preparations of bispecific IgGs. One way to achieve this is through the use of the common light chain principle, i.e. combining two binders that share one light chain but still have separate specificities. An exemplary method of enhancing the formation of a desired bispecific antibody from a mixture of monomers is by providing a common variable light chain to interact with each of the heteromeric variable heavy chain regions of the bispecific antibody. Compositions and methods of producing bispecific antibodies with a common light chain as disclosed in, e.g., U.S. Pat. No. 7,183,076B2, US20110177073A1, EP2847231A1, WO2016079081A1, and EP3055329A1, the contents of each of which is incorporated by reference herein.

### CrossMab

Another option to reduce light chain mispairing is the CrossMab technology which avoids non-specific L chain mispairing by exchanging CH1 and CL domains in the Fab of one half of the bispecific antibody. Such crossover variants retain binding specificity and affinity, but make the two arms so different that L chain mispairing is prevented. The CrossMab technology (as reviewed in Klein et al. Supra) involves domain swapping between heavy and light chains so as to promote the formation of the correct pairings. Briefly, to construct a bispecific IgG-like CrossMab antibody that could bind to two antigens by using two distinct light chain-heavy chain pairs, a two-step modification process is applied. First, a dimerization interface is engineered into the C-terminus of each heavy chain using a heterodimerization approach, e.g., Knob-into-hole (KiH) technology, to ensure that only a heterodimer of two distinct heavy chains from one antibody (e.g., Antibody A) and a second antibody (e.g., Antibody B) is efficiently formed. Next, the constant heavy 1 (CH1) and constant light (CL) domains of one antibody are exchanged (Antibody A), keeping the variable heavy (VH) and variable light (VL) domains consistent. The exchange of the CH1 and CL domains ensured that the modified antibody (Antibody A) light chain would only efficiently dimerize with the modified antibody (antibody A) heavy chain, while the unmodified antibody (Antibody B) light chain would only efficiently dimerize with the unmodified antibody (Antibody B) heavy chain; and thus only the desired bispecific CrossMab would be efficiently formed (see e.g., Cain, C. SciBX 4(28); doi:10.1038/scibx.2011.783, the contents of which are incorporated by reference herein).

### Common Heavy Chain

An exemplary method of enhancing the formation of a desired bispecific antibody from a mixture of monomers is by providing a common variable heavy chain to interact with each of the heteromeric variable light chain regions of the bispecific antibody. Compositions and methods of producing bispecific antibodies with a common heavy chain are disclosed in, e.g., US20120184716, US20130317200, and US20160264685A1, the contents of each of which is incorporated by reference herein.

### Amino Acid Modifications

Alternative compositions and methods of producing multispecific antibodies with correct light chain pairing include various amino acid modifications. For example, Zymeworks describes heterodimers with one or more amino acid modifications in the CH1 and/or CL domains, one or more amino acid modifications in the VH and/or VL domains, or a combination thereof, which are part of the interface between the light chain and heavy chain and create preferential pairing between each heavy chain and a desired light chain such that when the two heavy chains and two light chains of the heterodimer pair are co-expressed in a cell, the heavy chain of the first heterodimer preferentially pairs with one of the light chains rather than the other (see e.g., WO2015181805). Other exemplary methods are described in WO2016026943 (Argen-X), US20150211001, US20140072581A1, US20160039947A1, and US20150368352.

### Lambda/Kappa Formats

Multispecific molecules (e.g., multispecific antibody molecules) that include the lambda light chain polypeptide and a kappa light chain polypeptides, can be used to allow for heterodimerization. Methods for generating bispecific antibody molecules comprising the lambda light chain polypeptide and a kappa light chain polypeptides are disclosed in PCT/US17/53053 filed on Sep. 22, 2017, incorporated herein by reference in its entirety.

In embodiments, the multispecific molecules includes a multispecific antibody molecule, e.g., an antibody molecule comprising two binding specificities, e.g., a bispecific antibody molecule. The multispecific antibody molecule includes:
a lambda light chain polypeptide 1 (LLCP1) specific for a first epitope;
a heavy chain polypeptide 1 (HCP1) specific for the first epitope;
a kappa light chain polypeptide 2 (KLCP2) specific for a second epitope; and
a heavy chain polypeptide 2 (HCP2) specific for the second epitope.

“Lambda light chain polypeptide 1 (LLCP1)”, as that term is used herein, refers to a polypeptide comprising sufficient light chain (LC) sequence, such that when combined with a cognate heavy chain variable region, can mediate specific binding to its epitope and complex with an HCP1. In an embodiment it comprises all or a fragment of a CH1 region. In an embodiment, an LLCP1 comprises LC-CDR1, LC-CDR2, LC-CDR3, FR1, FR2, FR3, FR4, and CH1, or sufficient sequence therefrom to mediate specific binding of its epitope and complex with an HCP1. LLCP1, together with its HCP1, provide specificity for a first epitope (while KLCP2, together with its HCP2, provide specificity for a second epitope). As described elsewhere herein, LLCP1 has a higher affinity for HCP1 than for HCP2.

“Kappa light chain polypeptide 2 (KLCP2)”, as that term is used herein, refers to a polypeptide comprising sufficient light chain (LC) sequence, such that when combined with a cognate heavy chain variable region, can mediate specific binding to its epitope and complex with an HCP2. In an embodiment, it comprises all or a fragment of a CH1 region. In an embodiment, a KLCP2 comprises LC-CDR1, LC-CDR2, LC-CDR3, FR1, FR2, FR3, FR4, and CH1, or sufficient sequence therefrom to mediate specific binding of its epitope and complex with an HCP2. KLCP2, together with its HCP2, provide specificity for a second epitope (while LLCP1, together with its HCP1, provide specificity for a first epitope).

“Heavy chain polypeptide 1 (HCP1)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In an embodiment, it comprises all or a fragment of a CH1 region. In an embodiment, it comprises all or a fragment of a CH2 and/or CH3 region. In an embodiment an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an LLCP1, (ii) to complex preferentially, as described herein to LLCP1 as opposed to KLCP2; and (iii) to complex preferentially, as described herein, to an HCP2, as opposed to another molecule of HCP1. HCP1, together with its LLCP1, provide specificity for a first epitope (while KLCP2, together with its HCP2, provide specificity for a second epitope).

“Heavy chain polypeptide 2 (HCP2)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In an embodiment, it comprises all or a fragment of a CH1 region. In an embodiment, it comprises all or a fragment of a CH2 and/or CH3 region. In an embodiment an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an KLCP2, (ii) to complex preferentially, as described herein to KLCP2 as opposed to LLCP1; and (iii) to complex preferentially, as described herein, to an HCP1, as opposed to another molecule of HCP2. HCP2, together with its KLCP2, provide specificity for a second epitope (while LLCP1, together with its HCP1, provide specificity for a first epitope).

In some embodiments of the multispecific antibody molecule disclosed herein:
LLCP1 has a higher affinity for HCP1 than for HCP2; and/or
KLCP2 has a higher affinity for HCP2 than for HCP1.

In embodiments, the affinity of LLCP1 for HCP1 is sufficiently greater than its affinity for HCP2, such that under preselected conditions, e.g., in aqueous buffer, e.g., at pH 7, in saline, e.g., at pH 7, or under physiological conditions, at least 75%, 80, 90, 95, 98, 99, 99.5, or 99.9% of the multispecific antibody molecule molecules have a LLCP1 complexed, or interfaced with, a HCP1.

In some embodiments of the multispecific antibody molecule disclosed herein:
the HCP1 has a greater affinity for HCP2, than for a second molecule of HCP1; and/or
the HCP2 has a greater affinity for HCP1, than for a second molecule of HCP2.

In embodiments, the affinity of HCP1 for HCP2 is sufficiently greater than its affinity for a second molecule of HCP1, such that under preselected conditions, e.g., in aqueous buffer, e.g., at pH 7, in saline, e.g., at pH 7, or under physiological conditions, at least 75%, 80, 90, 95, 98, 99 99.5 or 99.9% of the multispecific antibody molecule molecules have a HCP1 complexed, or interfaced with, a HCP2.

In another aspect, disclosed herein is a method for making, or producing, a multispecific antibody molecule, e.g., as a first or second tumor-targeting moiety of a multispecific or multifunctional molecule polypeptide of the invention. The method includes:
(i) providing a first heavy chain polypeptide (e.g., a heavy chain polypeptide comprising one, two, three or all of a first heavy chain variable region (first VH), a first CH1, a first heavy chain constant region (e.g., a first CH2, a first CH3, or both));
(ii) providing a second heavy chain polypeptide (e.g., a heavy chain polypeptide comprising one, two, three or all of a second heavy chain variable region (second VH), a second CH1, a second heavy chain constant region (e.g., a second CH2, a second CH3, or both));
(iii) providing a lambda chain polypeptide (e.g., a lambda light variable region (VLλ), a lambda light constant chain (VLλ), or both) that preferentially associates with the first heavy chain polypeptide (e.g., the first VH); and
(iv) providing a kappa chain polypeptide (e.g., a lambda light variable region (VLκ), a lambda light constant chain (VLκ), or both) that preferentially associates with the second heavy chain polypeptide (e.g., the second VH),
under conditions where (i)-(iv) associate.

In embodiments, the first and second heavy chain polypeptides form an Fc interface that enhances heterodimerization.

In embodiments, (i)-(iv) (e.g., nucleic acid encoding (i)-(iv)) are introduced in a single cell, e.g., a single mammalian cell, e.g., a CHO cell. In embodiments, (i)-(iv) are expressed in the cell.

In embodiments, (i)-(iv) (e.g., nucleic acid encoding (i)-(iv)) are introduced in different cells, e.g., different mammalian cells, e.g., two or more CHO cell. In embodiments, (i)-(iv) are expressed in the cells.

In one embodiment, the method further comprises purifying a cell-expressed antibody molecule, e.g., using a lambda- and/or- kappa-specific purification, e.g., affinity chromatography.

In embodiments, the method further comprises evaluating the cell-expressed multispecific antibody molecule. For example, the purified cell-expressed multispecific antibody molecule can be analyzed by techniques known in the art, include mass spectrometry. In one embodiment, the purified cell-expressed antibody molecule is cleaved, e.g., digested with papain to yield the Fab moieties and evaluated using mass spectrometry.

In embodiments, the method produces correctly paired kappa/lambda multispecific, e.g., bispecific, antibody molecules in a high yield, e.g., at least 75, 80, 90, 95, 98, 99 99.5 or 99.9%.

In other embodiments, the multispecific, e.g., a bispecific, antibody molecule that includes:
(i) a first heavy chain polypeptide (HCP1) (e.g., a heavy chain polypeptide comprising one, two, three or all of a first heavy chain variable region (first VH), a first CH1, a first heavy chain constant region (e.g., a first CH2, a first CH3, or both)), e.g., wherein the HCP1 binds to a first epitope;
(ii) a second heavy chain polypeptide (HCP2) (e.g., a heavy chain polypeptide comprising one, two, three or all of a second heavy chain variable region (second VH), a second CH1, a second heavy chain constant region (e.g., a second CH2, a second CH3, or both)), e.g., wherein the HCP2 binds to a second epitope;
(iii) a lambda light chain polypeptide (LLCP1) (e.g., a lambda light variable region (VLl), a lambda light constant chain (VLl), or both) that preferentially associates with the first heavy chain polypeptide (e.g., the first VH), e.g., wherein the LLCP1 binds to a first epitope; and
(iv) a kappa light chain polypeptide (KLCP2) (e.g., a lambda light variable region (VLk), a lambda light constant chain (VLk), or both) that preferentially associates with the second heavy chain polypeptide (e.g., the second VH), e.g., wherein the KLCP2 binds to a second epitope.

In embodiments, the first and second heavy chain polypeptides form an Fc interface that enhances heterodimerization. In embodiments, the multispecific antibody molecule has a first binding specificity that includes a hybrid VLl-CLl heterodimerized to a first heavy chain variable region connected to the Fc constant, CH2-CH3 domain (having a knob modification) and a second binding specificity that includes a hybrid VLk-CLk heterodimerized to a second heavy chain variable region connected to the Fc constant, CH2-CH3 domain (having a hole modification).

### Tumor-Targeting Moieties

The present disclosure provides, inter alia, multispecific (e.g., bi-, tri-, tetra-specific) or multifunctional molecules, that include, e.g., are engineered to contain, one or more tumor-targeting moieties that bind to a tumor antigen, e.g., a tumor antigen chosen from: CD34, CD41, G6B, P-selectin, Clec2, cKIT, FLT3, MPL, ITGB3, ITGB2, GP5, GP6, GP9, GP1BA, DSC2, FCGR2A, TNFRSF10A, TNFRSF10B, or TM4SF1.

CD34 refers to hematopoietic progenitor cell antigen CD34. Swiss-Prot accession number P28906 provides exemplary human CD34 amino acid sequences. In some embodiments, CD34 or CD34 molecule is a naturally-existing CD34 or a functional variant or fragment thereof.

CD41 refers to ITGA2B, also known as Integrin alpha-IIb. Swiss-Prot accession number P08514 provides exemplary human CD41 amino acid sequences. In some embodiments, CD41 or CD41 molecule is a naturally-existing CD41 or a functional variant or fragment thereof.

G6B refers to MPIG6B, also known as megakaryocyte and platelet inhibitory receptor G6b or C6orf25. Swiss-Prot accession number 095866 provides exemplary human G6B amino acid sequences. In some embodiments, G6B or G6B molecule is a naturally-existing G6B or a functional variant or fragment thereof.

P-selectin refers to SELP, also known as CD62P, GMP-140 or LECAM3. Swiss-Prot accession number P16109 provides exemplary human P-selectin amino acid sequences. In some embodiments, P-selectin or P-selectin molecule is a naturally-existing P-selectin or a functional variant or fragment thereof.

Clec2 refers to CLEC1B, also known as C-type lectin domain family 1 member B. Swiss-Prot accession number Q9P126 provides exemplary human Clec2 amino acid sequences. In some embodiments, Clec2 or Clec2 molecule is a naturally-existing Clec2 or a functional variant or fragment thereof.

cKIT refers to mast/stem cell growth factor receptor kit, also known as CD117. Swiss-Prot accession number P10721 provides exemplary human cKIT amino acid sequences. In some embodiments, cKIT or cKIT molecule is a naturally-existing cKIT or a functional variant or fragment thereof.

FLT3 refers to receptor-type tyrosine-protein kinase FLT3, also known as CD135. Swiss-Prot accession number P36888 provides exemplary human FLT3 amino acid sequences. In some embodiments, FLT3 or FLT3 molecule is a naturally-existing FLT3 or a functional variant or fragment thereof.

MPL refers to thrombopoietin receptor, also known as CD110. Swiss-Prot accession number P40238 provides exemplary human MPL amino acid sequences. In some embodiments, MPL or MPL molecule is a naturally-existing MPL or a functional variant or fragment thereof.

ITGB3 refers to Integrin beta-3, also known as CD61. Swiss-Prot accession number P05106 provides exemplary human ITGB3 amino acid sequences. In some embodiments, ITGB3 or ITGB3 molecule is a naturally-existing ITGB3 or a functional variant or fragment thereof.

ITGB2 refers to Integrin beta-2, also known as CD18. Swiss-Prot accession number P05107 provides exemplary human ITGB2 amino acid sequences. In some embodiments, ITGB2 or ITGB2 molecule is a naturally-existing ITGB2 or a functional variant or fragment thereof.

GP5 refers to platelet glycoprotein V, also known as CD42d. Swiss-Prot accession number P40197 provides exemplary human GP5 amino acid sequences. In some embodiments, GP5 or GP5 molecule is a naturally-existing GP5 or a functional variant or fragment thereof.

GP6 refers to platelet glycoprotein VI. Swiss-Prot accession number Q9HCN6 provides exemplary human GP6 amino acid sequences. In some embodiments, GP6 or GP6 molecule is a naturally-existing GP6 or a functional variant or fragment thereof.

GP9 refers to platelet glycoprotein IX, also known as CD42a. Swiss-Prot accession number P14770 provides exemplary human GP9 amino acid sequences. In some embodiments, GP9 or GP9 molecule is a naturally-existing GP9 or a functional variant or fragment thereof.

GP1BA refers to platelet glycoprotein Ib alpha chain, also known as CD42b. Swiss-Prot accession number P07359 provides exemplary human GP1BA amino acid sequences. In some embodiments, GP1BA or GP1BA molecule is a naturally-existing GP1BA or a functional variant or fragment thereof.

DSC2 refers to desmocollin-2, also known as cadherin family member 2. Swiss-Prot accession number Q02487 provides exemplary human DSC2 amino acid sequences. In some embodiments, DSC2 or DSC2 molecule is a naturally-existing DSC2 or a functional variant or fragment thereof.

FCGR2A refers to Fc-gamma-RIIa, also known as CD32. Swiss-Prot accession number P12318 provides exemplary human FCGR2A amino acid sequences. In some embodiments, FCGR2A or FCGR2A molecule is a naturally-existing FCGR2A or a functional variant or fragment thereof.

TNFRSF10A refers to Tumor necrosis factor receptor superfamily member 10A, also known as Death receptor 4, TNF-related apoptosis-inducing ligand receptor 1, TRAIL-R1, or CD261. Swiss-Prot accession number 000220 provides exemplary human TNFRSF10A amino acid sequences. In some embodiments, TNFRSF10A or TNFRSF10A molecule is a naturally-existing TNFRSF10A or a functional variant or fragment thereof.

TNFRSF10B refers to Tumor necrosis factor receptor superfamily member 10B, also known as Death receptor 5, TNF-related apoptosis-inducing ligand receptor 2, TRAIL-R2, or CD262. Swiss-Prot accession number 014763 provides exemplary human TNFRSF10B amino acid sequences. In some embodiments, TNFRSF10B or TNFRSF10B molecule is a naturally-existing TNFRSF10B or a functional variant or fragment thereof.

TM4SF1 refers to transmembrane 4 L6 family member 1. Swiss-Prot accession number P30408 provides exemplary human TM4SF1 amino acid sequences. In some embodiments, TM4SF1 or TM4SF1 molecule is a naturally-existing TM4SF1 or a functional variant or fragment thereof.

In certain embodiments, the tumor antigen is CD34. In certain embodiments, the tumor antigen is CD41. In certain embodiments, the tumor antigen is G6B (also referred to herein as C6orf25). In certain embodiments, the tumor antigen is P-selectin. In certain embodiments, the tumor antigen is Clec2.

In some embodiments, the tumor-targeting moiety comprises an antibody, or an antigen-binding fragment thereof.

In some embodiments, the tumor-targeting moiety comprises a CDR, a framework region, or a variable region sequence shown in Table 2 (or a sequence having at least about 75%, 80%, 85%, 90%, 95%, or 99% sequence identity thereto).

TABLE 2
Sequences for exemplary antibodies capable of
binding to exemplary target molecules

| Target | Description | SEQ ID NO | Sequence |
|---|---|---|---|
| CD34 | Exemplary | SEQ ID NO: 1 | EVQLQQSGPELVKPGASVKISCKASGY |
| | anti-CD34 | | SFIGYFMNWVMQSHGRSLEWIGRINPY |
| | VH | | NGYTFYNQKFKGKATLTVDKSSSTAHM |
| | | | ELRSLASEDSAVYYCARHFRYDGVFYY |
| | | | AMDYWGQGTSVTVSS |
| | Exemplary | SEQ ID NO: 2 | QLVLTQSSSASFSLGASAKLTCTLSSQ |
| | anti-CD34 | | HSTFTIEWYQQQPLKPPKYVMDLKKDG |
| | VL | | SHSTGDGVPDRFSGSSSGADRYLSISN |
| | | | IQPEDEATYICGVGDTIKEQFVYVFGG |
| | | | GTKVTVL |
| cKIT | Exemplary | SEQ ID NO: 3 | EVQLVESGGGLVQPCIGSLRLSCAASG |
| (CD117) | anti-cKIT | | FAFSGYYMAWVRQAPGKGLEWVANINY |
| | VH | | PGSSTYYLDSVKGRFTISRDNAKNSLY |
| | | | LQMNSLRAEDTAVYYCARGDYYGTTYW |
| | | | YFDVWGQGTTVTVSS |
| | Exemplary | SEQ ID NO: 4 | DIQMTQSPSSLSASVGDRVTJTCRASQ |
| | anti-cKIT | | SISSYLNWYQQKPGKAPKLLIYYTSRL |
| | VL | | QSGVPSRFSGSGSGTDFTLTISSLQPE |
| | | | DFATYYCQQGRRLWSFGGGTKVEIK |
| FLT3 | Exemplary | SEQ ID NO: 5 | QVQLQQPGAELVKPGASLKLSCKSSGY |
| | anti-FLT3 | | TFTSYWMHWVRQRPGHGLEWIGEIDPS |
| | VH | | DSYKDYNQKFKDKATLTVDRSSNTAYM |
| | | | HLSSLTSDDSAVYYCARAITTTPFDFW |
| | | | GQGTTLTVSS |
| | Exemplary | SEQ ID NO: 6 | DIVLTQSPATLSVTPGDSVSLSCRASQ |
| | anti-FLT3 | | SISNNLHWYQQKSHESPRLLIKYASQS |
| | VL | | ISGIPSRFSGSGSGTDFTLSINSVETE |
| | | | DFGVYFCQQSNTWPYTFGGGTKLEIKR |
| CD41 | Exemplary | SEQ ID NO: 7 | EVQLQQSGAELVKPGASVKLSCTASGF |
| (ITGA2B) | anti-CD41 | | NIKDTYVHWVKQRPEQGLEWIGRIDPA |
| | VH | | NGYTKYDPKFQGKATITADTSSNTAYL |
| | | | QLSSLTSEDTAVYYCVRPLYDYYAMDY |
| | | | WGQGTSVTVSS |
| | Exemplary | SEQ ID NO: 8 | DILMTQSPSSMSVSLGDTVSITCHASQ |
| | anti-CD41 | | GISSNIGWLQQKPGKSFMGLIYYGTNL |
| | VL | | VDGVPSRFSGSGSGADYSLTISSLDSE |
| | | | DFADYYCVQYAQLPYTFGGGTKLEIK |
| MPL | 1.75 VH | SEQ ID NO: 9 | EVQLVESGGGLVQPKGSLKLSCAASGF |
| | | | SFNTYAMNWVRQAPGKGLEWIAHIRSK |
| | | | SNNFATYYADSVKDRFSISRDASENIL |
| | | | FLQMNNLKTEDTAMYYCVRQGGDFPMD |
| | | | YWGQGTSVTVSS |
| | 1.75 VL | SEQ ID NO: 10 | QIVLTQSPAIMSASPGEKVTISCSASS |
| | | | SVSYMYWYQQKPGSSPKPWIYRTSNLA |
| | | | SGVPARFSGSGSGTSYSLTISNMEAED |
| | | | AAAYYCQQYHSYPTTFGGGTKLEVK |
| | 1.78 VH | SEQ ID NO: 11 | QVQLQQSGPELVKPGASVKMSCKASGY |
| | | | AFSSSWLNWVRQRPGKGLEWIGRIYPG |
| | | | DGENHYNGKFKGKATLTADKSSSTGYM |
| | | | QLSSLTSEDSAVYFCASYYEGGYWGQG |
| | | | TLITVSA |
| | 1.78 VL | SEQ ID NO: 12 | DIVMTQAAPSIPVTPGESVSISCRSDK |
| | | | SLLHSNGNTYLFWFLQRPGQSPQLLIY |
| | | | RMSNLASGVPDRFSGSGSGTAFTLRIS |
| | | | GVEAEDVGVYYCMQHLEYPYTFGGGTK |
| | | | LEIK |
| P-Selctin | Exemplary | SEQ ID NO: 13 | EVQLVESGGGLVRPGGSLRLSCAASGF |
| (SELP) | anti-P- | | TFSNYDMHWVRQATGKGLEWVSAITAA |
| | Selectin | | GDIYYPGSVKGRFTISRENAKNSLYLQ |
| | VH | | MNSLRAGDTAVYYCARGRYSGSGSYYN |
| | | | DWFDPWGQGTLVTVSS |
| | Exemplary | SEQ ID NO: 14 | EIVLTQSPATLSLSPGERATLSCRASQ |
| | anti-P- | | SVSSYLAWYQQKPGQAPRLLIYDASNR |
| | Selectin | | ATGIPARFSGSGSGTDFTLTISSLEPE |
| | VL | | DFAVYYCQQRSNWPLTFGGGTKVEIK |
| DSC2 | Exemplary | SEQ ID NO: 15 | MDSRLNLVFLVLILKGVQCDVQLVESG |
| | anti-DSC2 | | GGLVQPGGSRKLSCAASGFTFSSFGMH |
| | #1 VH | | WVRQAPEKGLEWVAYISSGSSTIYYAD |
| | | | TVKGRFTISRDNPKNTLFLQMTSLRSE |
| | | | DTAMYYCARVHYYYFDYWGQGTTLTVS |
| | | | S |
| | Exemplary | SEQ ID NO: 16 | MRPSIQFLGLLLFWLHGAQCDIQMTQS |
| | anti-DSC2 | | PSSLSASLGGKVTITCKASQDINKYIA |
| | #1 VL | | WYQHKPGKGPRLLIHYTSTLQPGIPSR |
| | | | FSGSGSGRDYSFSISNLEPEDIATYYC |
| | | | LQYDNLWTFGGGTKL |
| | Exemplary | SEQ ID NO: 17 | MAWVWTLLFLMAAAQSIQAQIQLVQSG |
| | anti-DSC2 | | PELKKPGETVKISCKASGYTFTDYSMH |
| | #2 VH | | WVKQAPGKGLKWMGWINTETGEPTYAD |
| | | | DFKGRFAFSLETSASTAYLQINNLKNE |
| | | | DTATYFCARWLLFDYWGQGTTLTVSS |
| | Exemplary | SEQ ID NO: 18 | MESQTQVLMFLLLWVSGACADIVMTQS |
| | anti-DSC2 | | PSSLAMSVGQKVTMSCKSSQSLLNSSN |
| | #2 VL | | QKNYLAWYQQKPGQSPKLLVYFASTRE |
| | | | SGVPDRFIGSGSGTDFTLTISSVQAED |
| | | | LADYFCQQHYSTPLTFGAGTKL |
| FCGR2A | AT-10 VH | SEQ ID NO: 19 | EVKLEESGGGLVQPGGSMKLSCVASGF |
| (CD32a) | | | TFSYYWMNWVRQSPEKGLEWVAEIRLK |
| | | | SNNYATHYAESVKGRFTISRDDSKNNV |
| | | | YLQMNNLRAEDTGIYYCNRRDEYYAMD |
| | | | YWGQGTSVSVSS |
| | AT-10 VL | SEQ ID NO: 20 | DIVLTQSPGSLAVSLGQRATISCRASE |
| | | | SVDNFGISFMNWFQQKPGQPPRLLIYG |
| | | | ASNQGSGVPARFSGSGSGTDFSLNIHP |
| | | | VKKDDAAMYFCQQSKEVPWTFGGGTKL |
| | | | EIK |
| | IV.3 VH | SEQ ID NO: 21 | QIQLVQSGPELKKPGETVKISCKASGY |
| | | | TFTNYGMNWVKQAPGKGLKWMGWLNTY |
| | | | TGESIYPDDFKGRFAFSSETSASTAYL |
| | | | QINNLKNEDMATYFCARGDYGYDDPLD |
| | | | YWGQGTSVTVSS |
| | IV.3 VL | SEQ ID NO: 22 | DIVMTQAAPSVPVTPGESVSISCRSSK |
| | | | SLLHTNGNTYLHWFLQRPGQSPQLLIY |
| | | | RMSVLASGVPDRFSGSGSGTAFTLSIS |
| | | | RVEAEDVGVFYCMQHLEYPLTFGAGTK |
| | | | LELK |
| | MDE-8 VH | SEQ ID NO: 23 | QVHLVESGGGVVQPGRSLRLSCAASGF |
| | | | TFSSYGMHWVRQAPGKGLEWVAVIWYD |
| | | | GSNYYYTDSVKGRFTISRDNSKNTLYL |
| | | | QMNSLRAEDTAVYYCARDLGAAASDYW |
| | | | GQGTLVTVSS |
| | MDE-8 VL | SEQ ID NO: 24 | AIQLTQSPSSLSASVGDRVTITCRASQ |
| | | | GINSALAWYQQKPGKAPKLLIYDASSL |
| | | | ESGVPSRFSGSGSGTDFTLTISSLQPE |
| | | | DFATYYCQQFNSYPHTFGQGTKLEIK |
| TNFRSF10 | E-11-13 VH | SEQ ID NO: 25 | MDLMCKKMKHLWFFLLLVAAPRWVLSQ |
| A or | | | LQLQESGPGLVKPSETLSLTCTVSGGS |
| TNFRSF10 | | | IISKSSYWGWIRQPPGKGLEWIGSIYY |
| B | | | SGSTFYNPSLKSRVTISVDTSKNQFSL |
| | | | KLSSVTAADTAVYYCARLTVAEFDYWG |
| | | | QGTLVTVSSAS |
| | E-11-13 VL | SEQ ID NO: 26 | MEAPAQLLFLLLLWLPDTTGEIVLTQS |
| | | | PATLSLSPGERATLSCRASQSVSSFLA |
| | | | WYQQKPGQAPRLLIYDASNRATGIPAR |
| | | | FSGSGSGTDFTLTISSLEPEDFAVYYC |
| | | | QQRSNWPLTFGPGTKVDIKRT |
| | L-30-10 VH | SEQ ID NO: 27 | MDLMCKKMKHLWFFLLLVAAPRWVLSQ |
| | | | LQLQESGPGLVKPSETLSLTCTVSGGS |
| | | | ISSRSNYWGWIRQPPGKGLEWIGNVYY |
| | | | RGSTYYNSSLKSRVTISVDTSKNQFSL |
| | | | KLSSVTVADTAVYYCARLSVAEFDYWG |
| | | | QGILVTVSSAS |
| | L-30-10 VL | SEQ ID NO: 28 | MEAPAQLLFLLLLWLPDTTGEIVLTQS |
| | | | PATLSLSPGERATLSCRASQSVSSFLA |
| | | | WYQQKPGQAPRLLIYDASNRATGSPAR |
| | | | FSGSGSGTDFTLTISSLEPEDFAVYYC |
| | | | QQRSDWPLTFGPGTKVDIKRT |
| | H-48-2 VH | SEQ ID NO: 29 | MDLMCKKMKHLWFFLLLVAAPRWVLSQ |
| | | | LQLQESGPGLVKPSETLSLTCTVSGGS |
| | | | ISSSSYYWGWVRQPPGKGLEWIGSIHY |
| | | | SGSTFYNPSLKSRVTISVDTSKNQFSL |
| | | | KLSSVTAADTTVYYCARQGSTVVRGVY |
| | | | YYGMDVWGQGTTVTVSSAS |
| | H-48-2 VL | SEQ ID NO: 30 | METPAQLLFLLLLWLPDTTGEIVLTQS |
| | | | PGTLSLSPGERATLSCRASQSVSSSYL |
| | | | AWYQQKPGQAPRLLIYGASSRATGIPD |
| | | | RFSGSGSGTDFTLTISRLEPEDFAVYY |
| | | | CQQYGSSPLYTFGQGTKLEIKRT |
| | 0304 VH | SEQ ID NO: 31 | MDWTWRILFLVAAATSAHSQVQLVQSG |
| | | | AEMKKPGASVKVSCKTSGYTFTNYKIN |
| | | | WVRQAPGQGLEWMGWMNPDTDSTGYPQ |
| | | | KFQGRVTMTRNTSISTAYMELSSLRSE |
| | | | DTAVYYCARSYGSGSYYRDYYYGMDVW |
| | | | GQGTTVTVSS |
| | 0304 VL | SEQ ID NO: 32 | MEAPAQLLFLLLLWLPDTTGEIVLTQS |
| | | | PATLSLSPGERATLSCRASQSVSSYLA |
| | | | WYQQKPGQAPRLLIYDASNRATGIPAR |
| | | | FSGSGSGTDFTLTISSLEPEDFAVYYC |
| | | | QQRSNWPLTFGGGTKVEIKR |
| | KMTR1 VH | SEQ ID NO: 33 | MEFGLSWLFLVAILKGVQCEVQLLESG |
| | | | GGLVQPGRSLRLSCAASGFTFSSYAMS |
| | | | WVRQAPGKGLEWVSAISGSGGSRYYAD |
| | | | SVKGRFTISRDNSKNTLYLQMNSLRAE |
| | | | DTAVYYCAKESSGWFGAFDYWGQGTLV |
| | | | TVSS |
| | KMTR1 VL | SEQ ID NO: 34 | MSPSQLIGFLLLWVPASRGEIVLTQSP |
| | | | DFQSVTPKEKVTITCRASQSIGSSLHW |
| | | | YQQKPDQSPKLLIKYASQSFSGVPSRF |
| | | | SGSGSGTDFTLTINSLEAEDAAAYYCH |
| | | | QSSSLPITFGQGTRLEIKR |
| TM4SF1 | Exemplary | SEQ ID NO: 35 | EVILVESGGGLVKPGGSLKLSCAASGF |
| | anti- | | TFSSFAMSWVRQTPEKRLEWVATISSG |
| | TM4SF1 | | SIYIYYTDGVKGRFTISRDNAKNTVHL |
| | VH | | QMSSLRSEDTAMYYCARRGIYYGYDGY |
| | | | AMDYWGQGTSVTVSS |
| | Exemplary | SEQ ID NO: 36 | AVVMTQTPLSLPVSLGDQASISCRSSQ |
| | anti- | | SLVHSNGNTYLHWYMQKPGQSPKVLIY |
| | TM4SF1 | | KVSNRFSGVPDRFSGSGSGTDFTLKIS |
| | VL | | RVEADDLGIYFCSQSTHIPLAFGAGTK |
| | | | LELK |

### Exemplary Anti-CD34 Antibody Sequences

In one aspect, provided herein is a multispecific or multifunctional molecule comprising a tumor targeting moiety that comprises a CD34-targeting moiety. In another aspect, provided herein is an anti-CD34 antibody molecule (e.g., a monoclonal anti-CD34 antibody molecule).

In some embodiments, the CD34-targeting moiety or anti-CD34 antibody molecule comprises an antibody, or an antigen-binding fragment thereof, disclosed in Table 3 or Table 4. In some embodiments, the CD34-targeting moiety or anti-CD34 antibody molecule comprises a CDR, a framework region, or a variable region sequence disclosed in Table 3 or Table 4 (or a sequence having at least about 75%, 80%, 85%, 90%, 95%, or 99% sequence identity thereto).

In some embodiments, the CD34-targeting moiety or anti-CD34 antibody molecule comprises a VH comprising a heavy chain complementarity determining region 1 (VHCDR1) amino acid sequence of SEQ ID NO: 87 (or a sequence with no more than 1, 2, 3, or 4 mutations, e.g., substitutions, additions, or deletions), a VHCDR2 amino acid sequence of SEQ ID NO: 88 (or a sequence with no more than 1, 2, 3, or 4 mutations, e.g., substitutions, additions, or deletions), and/or a VHCDR3 amino acid sequence of SEQ ID NO: 89 (or a sequence with no more than 1, 2, 3, or 4 mutations, e.g., substitutions, additions, or deletions). In some embodiments, the CD34-targeting moiety or anti-CD34 antibody molecule comprises a VH comprising a VHCDR1 amino acid sequence of SEQ ID NO: 87, a VHCDR2 amino acid sequence of SEQ ID NO: 88, and/or a VHCDR3 amino acid sequence of SEQ ID NO: 89. In some embodiments, the CD34-targeting moiety or anti-CD34 antibody molecule comprises a VL comprising a light chain complementarity determining region 1 (VLCDR1) amino acid sequence of SEQ ID NO: 90 (or a sequence with no more than 1, 2, 3, or 4 mutations, e.g., substitutions, additions, or deletions), a VLCDR2 amino acid sequence of SEQ ID NO: 91 (or a sequence with no more than 1, 2, 3, or 4 mutations, e.g., substitutions, additions, or deletions), and/or a VLCDR3 amino acid sequence of SEQ ID NO: 92 (or a sequence with no more than 1, 2, 3, or 4 mutations, e.g., substitutions, additions, or deletions). In some embodiments, the CD34-targeting moiety or anti-CD34 antibody molecule comprises a VL comprising a VLCDR1 amino acid sequence of SEQ ID NO: 90, a VLCDR2 amino acid sequence of SEQ ID NO: 91, and a VLCDR3 amino acid sequence of SEQ ID NO: 92.

In some embodiments, the CD34-targeting moiety or anti-CD34 antibody molecule comprises a VH comprising the amino acid sequence of SEQ ID NO: 79, 80, 81, or 82, or an amino acid sequence having at least about 80%, 85%, 90%, 95%, or 99% sequence identity thereto. In some embodiments, the CD34-targeting moiety or anti-CD34 antibody molecule comprises a VL comprising the amino acid sequence of SEQ ID NO: 83, 84, 85, or 86, or an amino acid sequence having at least about 80%, 85%, 90%, 95%, or 99% sequence identity thereto. In some embodiments, the CD34-targeting moiety or anti-CD34 antibody molecule comprises a VH comprising the amino acid sequence of SEQ ID NO: 79 (or an amino acid sequence having at least about 80%, 85%, 90%, 95%, or 99% sequence identity thereto) and a VL comprising the amino acid sequence of SEQ ID NO: 83 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 79 and a VL comprising the amino acid sequence of SEQ ID NO: 83. In some embodiments, the CD34-targeting moiety or anti-CD34 antibody molecule comprises a VH comprising the amino acid sequence of SEQ ID NO: 80 (or an amino acid sequence having at least about 80%, 85%, 90%, 95%, or 99% sequence identity thereto) and a VL comprising the amino acid sequence of SEQ ID NO: 83 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 80 and a VL comprising the amino acid sequence of SEQ ID NO: 83. In some embodiments, the CD34-targeting moiety or anti-CD34 antibody molecule comprises a VH comprising the amino acid sequence of SEQ ID NO: 81 (or an amino acid sequence having at least about 80%, 85%, 90%, 95%, or 99% sequence identity thereto) and a VL comprising the amino acid sequence of SEQ ID NO: 83 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 81 and a VL comprising the amino acid sequence of SEQ ID NO: 83. In some embodiments, the CD34-targeting moiety or anti-CD34 antibody molecule comprises a VH comprising the amino acid sequence of SEQ ID NO: 82 (or an amino acid sequence having at least about 80%, 85%, 90%, 95%, or 99% sequence identity thereto) and a VL comprising the amino acid sequence of SEQ ID NO: 83 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 82 and a VL comprising the amino acid sequence of SEQ ID NO: 83. In some embodiments, the CD34-targeting moiety or anti-CD34 antibody molecule comprises a VH comprising the amino acid sequence of SEQ ID NO: 79 (or an amino acid sequence having at least about 80%, 85%, 90%, 95%, or 99% sequence identity thereto) and a VL comprising the amino acid sequence of SEQ ID NO: 84 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 79 and a VL comprising the amino acid sequence of SEQ ID NO: 84. In some embodiments, the CD34-targeting moiety or anti-CD34 antibody molecule comprises a VH comprising the amino acid sequence of SEQ ID NO: 80 (or an amino acid sequence having at least about 80%, 85%, 90%, 95%, or 99% sequence identity thereto) and a VL comprising the amino acid sequence of SEQ ID NO: 84 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 80 and a VL comprising the amino acid sequence of SEQ ID NO: 84. In some embodiments, the CD34-targeting moiety or anti-CD34 antibody molecule comprises a VH comprising the amino acid sequence of SEQ ID NO: 81 (or an amino acid sequence having at least about 80%, 85%, 90%, 95%, or 99% sequence identity thereto) and a VL comprising the amino acid sequence of SEQ ID NO: 84 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 81 and a VL comprising the amino acid sequence of SEQ ID NO: 84. In some embodiments, the CD34-targeting moiety or anti-CD34 antibody molecule comprises a VH comprising the amino acid sequence of SEQ ID NO: 82 (or an amino acid sequence having at least about 80%, 85%, 90%, 95%, or 99% sequence identity thereto) and a VL comprising the amino acid sequence of SEQ ID NO: 84 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 82 and a VL comprising the amino acid sequence of SEQ ID NO: 84. In some embodiments, the CD34-targeting moiety or anti-CD34 antibody molecule comprises a VH comprising the amino acid sequence of SEQ ID NO: 79 (or an amino acid sequence having at least about 80%, 85%, 90%, 95%, or 99% sequence identity thereto) and a VL comprising the amino acid sequence of SEQ ID NO: 85 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 79 and a VL comprising the amino acid sequence of SEQ ID NO: 85. In some embodiments, the CD34-targeting moiety or anti-CD34 antibody molecule comprises a VH comprising the amino acid sequence of SEQ ID NO: 80 (or an amino acid sequence having at least about 80%, 85%, 90%, 95%, or 99% sequence identity thereto) and a VL comprising the amino acid sequence of SEQ ID NO: 85 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 80 and a VL comprising the amino acid sequence of SEQ ID NO: 85. In some embodiments, the CD34-targeting moiety or anti-CD34 antibody molecule comprises a VH comprising the amino acid sequence of SEQ ID NO: 81 (or an amino acid sequence having at least about 80%, 85%, 90%, 95%, or 99% sequence identity thereto) and a VL comprising the amino acid sequence of SEQ ID NO: 85 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 81 and a VL comprising the amino acid sequence of SEQ ID NO: 85. In some embodiments, the CD34-targeting moiety or anti-CD34 antibody molecule comprises a VH comprising the amino acid sequence of SEQ ID NO: 82 (or an amino acid sequence having at least about 80%, 85%, 90%, 95%, or 99% sequence identity thereto) and a VL comprising the amino acid sequence of SEQ ID NO: 85 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 82 and a VL comprising the amino acid sequence of SEQ ID NO: 85. In some embodiments, the CD34-targeting moiety or anti-CD34 antibody molecule comprises a VH comprising the amino acid sequence of SEQ ID NO: 79 (or an amino acid sequence having at least about 80%, 85%, 90%, 95%, or 99% sequence identity thereto) and a VL comprising the amino acid sequence of SEQ ID NO: 86 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 79 and a VL comprising the amino acid sequence of SEQ ID NO: 86. In some embodiments, the CD34-targeting moiety or anti-CD34 antibody molecule comprises a VH comprising the amino acid sequence of SEQ ID NO: 80 (or an amino acid sequence having at least about 80%, 85%, 90%, 95%, or 99% sequence identity thereto) and a VL comprising the amino acid sequence of SEQ ID NO: 86 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 80 and a VL comprising the amino acid sequence of SEQ ID NO: 86. In some embodiments, the CD34-targeting moiety or anti-CD34 antibody molecule comprises a VH comprising the amino acid sequence of SEQ ID NO: 81 (or an amino acid sequence having at least about 80%, 85%, 90%, 95%, or 99% sequence identity thereto) and a VL comprising the amino acid sequence of SEQ ID NO: 86 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 81 and a VL comprising the amino acid sequence of SEQ ID NO: 86. In some embodiments, the CD34-targeting moiety or anti-CD34 antibody molecule comprises a VH comprising the amino acid sequence of SEQ ID NO: 82 (or an amino acid sequence having at least about 80%, 85%, 90%, 95%, or 99% sequence identity thereto) and a VL comprising the amino acid sequence of SEQ ID NO: 86 (or an amino acid sequence having at least about 80%, 85%, 90%, 95%, or 99% sequence identity thereto). In some embodiments, the CD34-targeting moiety or anti-CD34 antibody molecule comprises a VH comprising the amino acid sequence of SEQ ID NO: 82 and a VL comprising the amino acid sequence of SEQ ID NO: 86.

In some embodiments, the CD34-targeting moiety or anti-CD34 antibody molecule comprises a VH comprising the amino acid sequence of SEQ ID NO: 79 and a VL comprising the amino acid sequence of SEQ ID NO: 84.

TABLE 3
Exemplary variable region sequences of anti-CD34 antibodies

| SEQ ID NO | Description | Sequence |
|---|---|---|
| SEQ ID NO: | Mouse VH | EIQLQQSGPELMKPGASLKISCKTSGYSFTSYYMHWVKQSHG |
| 77 | | QSLEWIGFIDPFKVITGYNHNFRGKATLTVDRSSTTAYMHLR |
| | | SLTSEDSAVYYCARRYYSDYDGYALDYWGQGTSVTVSS |
| SEQ ID NO: | Mouse VL | DVVMTQTPLSLPVSLGDQASIFCRSSQSLVHSDGNTYLHWYL |
| 78 | | QKPGQSPKLLIYKVSNRFSGVPDRFSGSGSGTDFTLKISRVE |
| | | AEDLGVYFCSQSTHVPPYTFGGGTKLEIK |
| SEQ ID NO: | Humanization | QIQLQESGPGLVKPSETLSLTCTTSGYSFTSYYMHWIRQPPG |
| 79 | variant VH1 | KGLEWIGFIDPFKVITGYNHNFRGRVTISVDRSKTQASLKLS |
| | | SVTAADTAVYYCARRYYSDYDGYALDYWGQGTLVTVSS |
| SEQ ID NO: | Humanization | EIQLVQSGAEVKKPGATVKISCKTSGYSFTSYYMHWVQQAPG |
| 80 | variant VH2 | KGLEWMGFIDPFKVITGYNHNFRGRVTITVDRSTTTAYMELS |
| | | SLRSEDTAVYYCARRYYSDYDGYALDYWGQGTLVTVSS |
| SEQ ID NO: | Humanization | QIQLVQSGAEVKKTGSSVKVSCKTSGYSFTSYYMHWVRQAPG |
| 81 | variant VH3 | QALEWMGFIDPFKVITGYNHNFRGRVTITVDRSMTTAYMELS |
| | | SLRSEDTAMYYCARRYYSDYDGYALDYWGQGTLVTVSS |
| SEQ ID NO: | Humanization | QIQLVQSGAEVKKPGASVKVSCKTSGYSFTSYYMHWVRQAPG |
| 82 | variant VH4 | QGLEWMGFIDPFKVITGYNHNFRGRVTSTVDRSITTAYMELS |
| | | RLRSDDTVVYYCARRYYSDYDGYALDYWGQGTLVTVSS |
| SEQ ID NO: | Humanization | EVVMTQSPGTLSLSPGERATLSCRSSQSLVHSDGNTYLHWYQ |
| 83 | variant VL1 | QKPGQAPRLLIYKVSNRFSGIPDRFSGSGSGTDFTLTISRLE |
| | | PEDFAVYFCSQSTHVPPYTFGGGTKVEIK |
| SEQ ID NO: | Humanization | EVVMTQSPATLSLSPGERATLSCRSSQSLVHSDGNTYLHWYQ |
| 84 | variant VL2 | QKPGQAPRLLIYKVSNRFSGIPARFSGSGSGTDFTLTISSLE |
| | | PEDFAVYFCSQSTHVPPYTFGGGTKVEIK |
| SEQ ID NO: | Humanization | EVVMTQSPATLSVSPGERATLSCRSSQSLVHSDGNTYLHWYQ |
| 85 | variant VL3 | QKPGQAPRLLIYKVSNRFSGIPARFSGSGSGTEFTLTISSLQ |
| | | SEDFAVYFCSQSTHVPPYTFGGGTKVEIK |
| SEQ ID NO: | Humanization | VVWMTQSPSLLSASTGDRVTISCRSSQSLVHSDGNTYLHWYQ |
| 86 | variant VL4 | QKPGKAPELLIYKVSNRFSGVPSRFSGSGSGTDFTLTISCLQ |
| | | SEDFATYFCSQSTHVPPYTFGGGTKVEIK |

TABLE 4
Exemplary CDRs of anti-CD34 antibodies

| SEQ ID NO | Description | Sequence |
|---|---|---|
| SEQ ID NO: 87 | VH CDR1 | FTSYYMH |
| SEQ ID NO: 88 | VH CDR2 | FIDPFKVITGYNHNFRG |
| SEQ ID NO: 89 | VH CDR3 | RYYSDYDGYALDY |
| SEQ ID NO: 90 | VL CDR1 | RSSQSLVHSDGNTYLH |
| SEQ ID NO: 91 | VL CDR2 | KVSNRFS |
| SEQ ID NO: 92 | VL CDR3 | SQSTHVPPYT |

### TGF-Beta Inhibitor

In one aspect, provided herein is a multispecific antibody molecule comprising a TGF-beta inhibitor. In some embodiments, the TGF-beta inhibitor binds to and inhibits TGF-beta, e.g., reduces the activity of TGF-beta. In some embodiments, the TGF-beta inhibitor inhibits (e.g., reduces the activity of) TGF-beta 1. In some embodiments, the TGF-beta inhibitor inhibits (e.g., reduces the activity of) TGF-beta 2. In some embodiments, the TGF-beta inhibitor inhibits (e.g., reduces the activity of) TGF-beta 3. In some embodiments, the TGF-beta inhibitor inhibits (e.g., reduces the activity of) TGF-beta 1 and TGF-beta 3. In some embodiments, the TGF-beta inhibitor inhibits (e.g., reduces the activity of) TGF-beta 1, TGF-beta 2, and TGF-beta 3.

In some embodiments, the TGF-beta inhibitor comprises a portion of a TGF-beta receptor (e.g., an extracellular domain of a TGF-beta receptor) that is capable of inhibiting (e.g., reducing the activity of) TGF-beta, or functional fragment or variant thereof. In some embodiments, the TGF-beta inhibitor comprises a TGFBR1 polypeptide (e.g., an extracellular domain of TGFBR1 or functional variant thereof). In some embodiments, the TGF-beta inhibitor comprises a TGFBR2 polypeptide (e.g., an extracellular domain of TGFBR2 or functional variant thereof). In some embodiments, the TGF-beta inhibitor comprises a TGFBR3 polypeptide (e.g., an extracellular domain of TGFBR3 or functional variant thereof). In some embodiments, the TGF-beta inhibitor comprises a TGFBR1 polypeptide (e.g., an extracellular domain of TGFBR1 or functional variant thereof) and a TGFBR2 polypeptide (e.g., an extracellular domain of TGFBR2 or functional variant thereof). In some embodiments, the TGF-beta inhibitor comprises a TGFBR1 polypeptide (e.g., an extracellular domain of TGFBR1 or functional variant thereof) and a TGFBR3 polypeptide (e.g., an extracellular domain of TGFBR3 or functional variant thereof). In some embodiments, the TGF-beta inhibitor comprises a TGFBR2 polypeptide (e.g., an extracellular domain of TGFBR2 or functional variant thereof) and a TGFBR3 polypeptide (e.g., an extracellular domain of TGFBR3 or functional variant thereof).

Exemplary TGF-beta receptor polypeptides that can be used as TGF-beta inhibitors have been disclosed in U.S. Pat. Nos. 8,993,524, 9,676,863, 8,658,135, US20150056199, US20070184052, and WO2017037634, all of which are herein incorporated by reference in their entirety.

In some embodiments, the TGF-beta inhibitor comprises an extracellular domain of TGFBR1 or a sequence substantially identical thereto (e.g., a sequence that is at least 80%, 85%, 90%, or 95% identical thereto). In some embodiments, the TGF-beta inhibitor comprises an extracellular domain of SEQ ID NO: 95, or a sequence substantially identical thereto (e.g., a sequence that is at least 80%, 85%, 90%, or 95% identical thereto). In some embodiments, the TGF-beta inhibitor comprises an extracellular domain of SEQ ID NO: 96, or a sequence substantially identical thereto (e.g., a sequence that is at least 80%, 85%, 90%, or 95% identical thereto). In some embodiments, the TGF-beta inhibitor comprises an extracellular domain of SEQ ID NO: 97, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 104, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 105, or a sequence substantially identical thereto (e.g., a sequence that is at least 80%, 85%, 90%, or 95% identical thereto).

In some embodiments, the TGF-beta inhibitor comprises an extracellular domain of TGFBR2 or a sequence substantially identical thereto (e.g., a sequence that is at least 80%, 85%, 90%, or 95% identical thereto). In some embodiments, the TGF-beta inhibitor comprises an extracellular domain of SEQ ID NO: 98, or a sequence substantially identical thereto (e.g., a sequence that is at least 80%, 85%, 90%, or 95% identical thereto). In some embodiments, the TGF-beta inhibitor comprises an extracellular domain of SEQ ID NO: 99, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 100, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 101, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 102, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 103, or a sequence substantially identical thereto (e.g., a sequence that is at least 80%, 85%, 90%, or 95% identical thereto).

In some embodiments, the TGF-beta inhibitor comprises an extracellular domain of TGFBR3 or a sequence substantially identical thereto (e.g., a sequence that is at least 80%, 85%, 90%, or 95% identical thereto). In some embodiments, the TGF-beta inhibitor comprises an extracellular domain of SEQ ID NO: 106, or a sequence substantially identical thereto (e.g., a sequence that is at least 80%, 85%, 90%, or 95% identical thereto). In some embodiments, the TGF-beta inhibitor comprises an extracellular domain of SEQ ID NO: 107, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 108, or a sequence substantially identical thereto (e.g., a sequence that is at least 80%, 85%, 90%, or 95% identical thereto).

In some embodiments, the TGF-beta inhibitor comprises no more than one TGF-beta receptor extracellular domain. In some embodiments, the TGF-beta inhibitor comprises two or more (e.g., two, three, four, five, or more) TGF-beta receptor extracellular domains, linked together, e.g., via a linker.

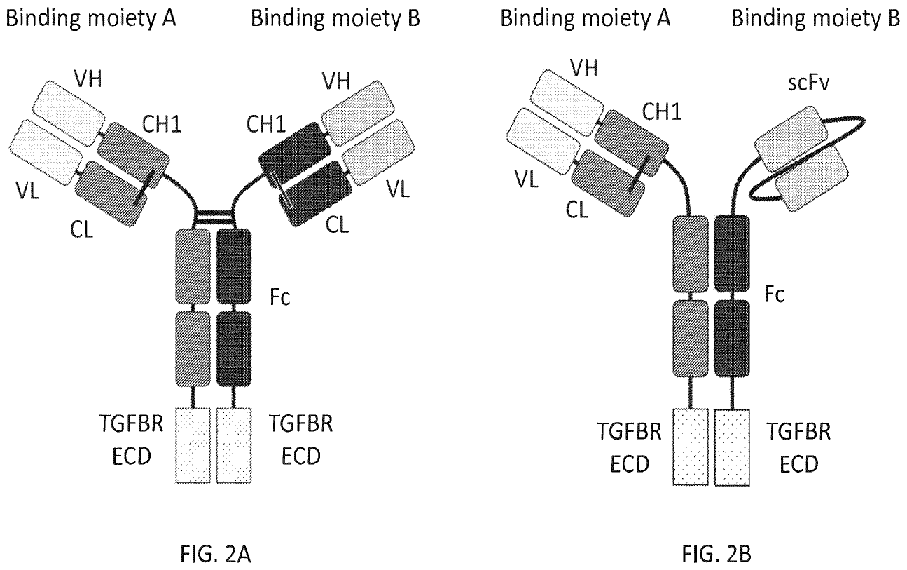

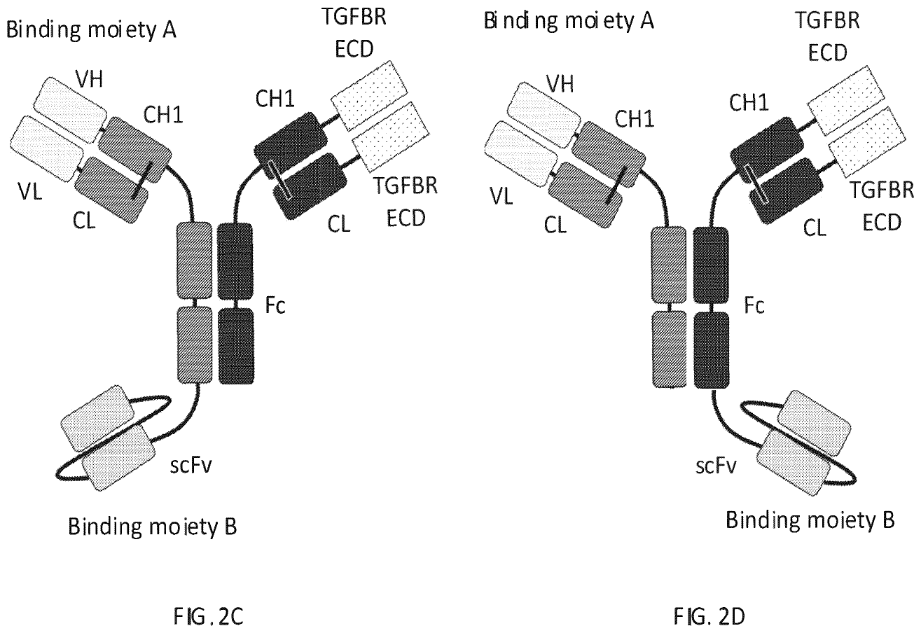

In some embodiments, the multispecific molecule comprises a configuration shown in FIGS. 2A-2D. In some embodiments, the TGFβ inhibitor comprises a TGF-beta receptor ECD homodimer. In some embodiments, the TGFβ inhibitor comprises a TGF-beta receptor ECD heterodimer. In some embodiments, the two TGFBR ECD domains are linked to two Fc regions, e.g., the C-terminus of two Fc regions. In some embodiments, the two TGFBR ECD domains are linked to CH1 and CL, respectively.

TABLE 5
Exemplary amino acid sequences of TGF-beta
polypeptides or TGF-beta receptor polypeptides

| SEQ ID | | |
|---|---|---|
| NO | Description | Amino acid sequence |
| SEQ ID | Immature | MPPSGLRLLLLLLPLLWLLVLTPGRPAAGLSTCKTIDMELVKRKRIE |
| NO: 200 | human | AIRGQILSKLRLASPPSQGEVPPGPLPEAVLALYNSTRDRVAGESAE |
| | TGF-beta 1 | PEPEPEADYYAKEVTRVLMVETHNEIYDKFKQSTHSIYMFFNTSELR |
| | (P01137-1) | EAVPEPVLLSRAELRLLRLKLKVEQHVELYQKYSNNSWRYLSNRLLA |
| | | PSDSPEWLSFDVTGVVRQWLSRGGEIEGFRLSAHCSCDSRDNTLQVD |
| | | INGFTTGRRGDLATIHGMNRPFLLLMATPLERAQHLQSSRHRRALDT |
| | | NYCFSSTEKNCCVRQLYIDFRKDLGWKWIHEPKGYHANFCLGPCPYI |
| | | WSLDTQYSKVLALYNQHNPGASAAPCCVPQALEPLPIVYYVGRKPK |
| | | VEQLSNMIVRSCKCS |
| SEQ ID | Human | LSTCKTIDMELVKRKRIEAIRGQILSKLRLASPPSQGEVPPGPLPEA |
| NO: 117 | TGF-beta 1 | VLALYNSTRDRVAGESAEPEPEPEADYYAKEVTRVLMVETHNEIYDK |
| | (P01137-1) | FKQSTHSIYMFFNTSELREAVPEPVLLSRAELRLLRLKLKVEQHVEL |
| | | YQKYSNNSWRYLSNRLLAPSDSPEWLSFDVTGVVRQWLSRGGEIEGF |
| | | RLSAHCSCDSRDNTLQVDINGFTTGRRGDLATIHGMNRPFLLLMATP |
| | | LERAQHLQSSRHRRALDTNYCFSSTEKNCCVRQLYIDFRKDLGWKWI |
| | | HEPKGYHANFCLGPCPYIWSLDTQYSKVLALYNQHNPGASAAPCCVP |
| | | QALEPLPIVYYVGRKPKVEQLSNMIVRSCKCS |
| SEQ ID | Immature | MHYCVLSAFLILHLVTVALSLSTCSTLDMDQFMRKRIEAIRGQILSK |
| NO: 93 | human | LKLTSPPEDYPEPEEVPPEVISIYNSTRDLLQEKASRRAAACERERS |
| | TGF-beta 2 | DEEYYAKEVYKIDMPPFFPSENAIPPTFYRPYFRIVRFDVSAMEKNA |
| | (P61812-1) | SNLVKAEFRVFRLQNPKARVPEQRIELYQILKSKDLTSPTQRYIDSK |
| | | VVKTRAEGEWLSFDVTDAVHEWLHHKDRNLGFKISLHCPCCTFVPSN |
| | | NYIIPNKSEELEARFAGIDGTSTYTSGDQKTIKSTRKKNSGKTPHLL |
| | | LMLLPSYRLESQQTNRRKKRALDAAYCFRNVQDNCCLRPLYIDFKRD |
| | | LGWKWIHEPKGYNANFCAGACPYLWSSDTQHSRVLSLYNTINPEASA |
| | | SPCCVSQDLEPLTILYYIGKTPKIEQLSNMIVKSCKCS |
| SEQ ID | Human | LSTCSTLDMDQFMRKRIEAIRGQILSKLKLTSPPEDYPEPEEVPPEV |
| NO: 118 | TGF-beta 2 | ISIYNSTRDLLQEKASRRAAACERERSDEEYYAKEVYKIDMPPFFPS |
| | (P61812-1) | ENAIPPTFYRPYFRIVRFDVSAMEKNASNLVKAEFRVFRLQNPKARV |
| | | PEQRIELYQILKSKDLTSPTQRYIDSKVVKTRAEGEWLSFDVTDAVH |
| | | EWLHHKDRNLGFKISLHCPCCTFVPSNNYIIPNKSEELEARFAGIDG |
| | | TSTYTSGDQKTIKSTRKKNSGKTPHLLLMLLPSYRLESQQTNRRKKR |
| | | ALDAAYCFRNVQDNCCLRPLYIDFKRDLGWKWIHEPKGYNANFCAGA |
| | | CPYLWSSDTQHSRVLSLYNTINPEASASPCCVSQDLEPLTILYYIGK |
| | | TPKIEQLSNMIVKSCKCS |
| SEQ ID | Immature | MKMHLQRALVVLALLNFATVSLSLSTCTTLDFGHIKKKRVEAIRGQI |
| NO: 94 | human | LSKLRLTSPPEPTVMTHVPYQVLALYNSTRELLEEMHGEREEGCTQE |
| | TGF-beta 3 | NTESEYYAKEIHKFDMIQGLAEHNELAVCPKGITSKVFRFNVSSVEK |
| | (P10600-1) | NRTNLFRAEFRVLRVPNPSSKRNEQRIELFQILRPDEHIAKQRYIGG |
| | | KNLPTRGTAEWLSFDVTDTVREWLLRRESNLGLEISIHCPCHTFQPN |
| | | GDILENIHEVMEIKFKGVDNEDDHGRGDLGRLKKQKDHHNPHLILMM |
| | | IPPHRLDNPGQGGQRKKRALDTNYCFRNLEENCCVRPLYIDFRQDLG |
| | | WKWVHEPKGYYANFCSGPCPYLRSADTTHSTVLGLYNTLNPEASASP |
| | | CCVPQDLEPLTILYYVGRTPKVEQLSNMVVKSCKCS |
| SEQ ID | Human | LSTCTTLDFGHIKKKRVEAIRGQILSKLRLTSPPEPTVMTHVPYQVL |
| NO: 119 | TGF-beta 3 | ALYNSTRELLEEMHGEREEGCTQENTESEYYAKEIHKFDMIQGLAEH |
| | (P10600-1) | NELAVCPKGITSKVFRFNVSSVEKNRTNLFRAEFRVLRVPNPSSKRN |
| | | EQRIELFQILRPDEHIAKQRYIGGKNLPTRGTAEWLSFDVTDTVREW |
| | | LLRRESNLGLEISIHCPCHTFQPNGDILENIHEVMEIKFKGVDNEDD |
| | | HGRGDLGRLKKQKDHHNPHLILMMIPPHRLDNPGQGGQRKKRALDTN |
| | | YCFRNLEENCCVRPLYIDFRQDLGWKWVHEPKGYYANFCSGPCPYLR |
| | | SADTTHSTVLGLYNTLNPEASASPCCVPQDLEPLTILYYVGRTPKVE |
| | | QLSNMVVKSCKCS |
| SEQ ID | Immature | MEAAVAAPRPRLLLLVLAAAAAAAAALLPGATALQCFCHLCTKDNFT |
| NO: 95 | human | CVTDGLCFVSVTETTDKVIHNSMCIAEIDLIPRDRPFVCAPSSKTGS |
| | TGFBR1 | VTTTYCCNQDHCNKIELPTTVKSSPGLGPVELAAVIAGPVCFVCISL |
| | isoform 1 | MLMVYICHNRTVIHHRVPNEEDPSLDRPFISEGTTLKDLIYDMTTSG |
| | (P36897-1) | SGSGLPLLVQRTIARTIVLQESIGKGRFGEVWRGKWRGEEVAVKIFS |
| | | SREERSWFREAEIYQTVMLRHENILGFIAADNKDNGTWTQLWLVSDY |
| | | HEHGSLFDYLNRYTVTVEGMIKLALSTASGLAHLHMEIVGTQGKPAI |
| | | AHRDLKSKNILVKKNGTCCIADLGLAVRHDSATDTIDIAPNHRVGTK |
| | | RYMAPEVLDDSINMKHFESFKRADIYAMGLVFWEIARRCSIGGIHED |
| | | YQLPYYDLVPSDPSVEEMRKVVCEQKLRPNIPNRWQSCEALRVMAKI |
| | | MRECWYANGAARLTALRIKKTLSQLSQQEGIKM |
| SEQ ID | Human | LQCFCHLCTKDNFTCVTDGLCFVSVTETTDKVIHNSMCIAEIDLIPR |
| NO: 120 | TGFBR1 | DRPFVCAPSSKTGSVTTTYCCNQDHCNKIELPTTVKSSPGLGPVELA |
| | isoform 1 | AVIAGPVCFVCISLMLMVYICHNRTVIHHRVPNEEDPSLDRPFISEG |
| | (P36897-1) | TTLKDLIYDMTTSGSGSGLPLLVQRTIARTIVLQESIGKGRFGEVWR |
| | | GKWRGEEVAVKIFSSREERSWFREAEIYQTVMLRHENILGFIAADNK |
| | | DNGTWTQLWLVSDYHEHGSLFDYLNRYTVTVEGMIKLALSTASGLAH |
| | | LHMEIVGTQGKPAIAHRDLKSKNILVKKNGTCCIADLGLAVRHDSAT |
| | | DTIDIAPNHRVGTKRYMAPEVLDDSINMKHFESFKRADIYAMGLVFW |
| | | EIARRCSIGGIHEDYQLPYYDLVPSDPSVEEMRKVVCEQKLRPNIPN |
| | | RWQSCEALRVMAKIMRECWYANGAARLTALRIKKTLSQLSQQEGIKM |
| SEQ ID | Immature | MEAAVAAPRPRLLLLVLAAAAAAAAALLPGATALQCFCHLCTKDNFT |
| NO: 96 | human | CVTDGLCFVSVTETTDKVIHNSMCIAEIDLIPRDRPFVCAPSSKTGS |
| | TGFBR1 | VTTTYCCNQDHCNKIELPTTGPFSVKSSPGLGPVELAAVIAGPVCFV |
| | isoform 2 | CISLMLMVYICHNRTVIHHRVPNEEDPSLDRPFISEGTTLKDLIYDM |
| | (P36897-2) | TTSGSGSGLPLLVQRTIARTIVLQESIGKGRFGEVWRGKWRGEEVAV |
| | | KIFSSREERSWFREAEIYQTVMLRHENILGFIAADNKDNGTWTQLWL |
| | | VSDYHEHGSLFDYLNRYTVTVEGMIKLALSTASGLAHLHMEIVGTQG |
| | | KPAIAHRDLKSKNILVKKNGTCCIADLGLAVRHDSATDTIDIAPNHR |
| | | VGTKRYMAPEVLDDSINMKHFESFKRADIYAMGLVFWEIARRCSIGG |
| | | IHEDYQLPYYDLVPSDPSVEEMRKVVCEQKLRPNIPNRWQSCEALRV |
| | | MAKIMRECWYANGAARLTALRIKKTLSQLSQQEGIKM |
| SEQ ID | Human | LQCFCHLCTKDNFTCVTDGLCFVSVTETTDKVIHNSMCIAEIDLIPR |
| NO: 121 | TGFBR1 | DRPFVCAPSSKTGSVTTTYCCNQDHCNKIELPTTGPFSVKSSPGLGP |
| | isoform 2 | VELAAVIAGPVCFVCISLMLMVYICHNRTVIHHRVPNEEDPSLDRPF |
| | (P36897-2) | ISEGTTLKDLIYDMTTSGSGSGLPLLVQRTIARTIVLQESIGKGRFG |
| | | EVWRGKWRGEEVAVKIFSSREERSWFREAEIYQTVMLRHENILGFIA |
| | | ADNKDNGTWTQLWLVSDYHEHGSLFDYLNRYTVTVEGMIKLALSTAS |
| | | GLAHLHMEIVGTQGKPAIAHRDLKSKNILVKKNGTCCIADLGLAVRH |
| | | DSATDTIDIAPNHRVGTKRYMAPEVLDDSINMKHFESFKRADIYAMG |
| | | LVFWEIARRCSIGGIHEDYQLPYYDLVPSDPSVEEMRKVVCEQKLRP |
| | | NIPNRWQSCEALRVMAKIMRECWYANGAARLTALRIKKTLSQLSQQE |
| | | GIKM |
| SEQ ID | Immature | MEAAVAAPRPRLLLLVLAAAAAAAAALLPGATALQCFCHLCTKDNFT |
| NO: 97 | human | CVTDGLCFVSVTETTDKVIHNSMCIAEIDLIPRDRPFVCAPSSKTGS |
| | TGFBR1 | VTTTYCCNQDHCNKIELPTTGLPLLVQRTIARTIVLQESIGKGRFGE |
| | isoform 3 | VWRGKWRGEEVAVKIFSSREERSWFREAEIYQTVMLRHENILGFIAA |
| | (P36897-3) | DNKDNGTWTQLWLVSDYHEHGSLFDYLNRYTVTVEGMIKLALSTASG |
| | | LAHLHMEIVGTQGKPAIAHRDLKSKNILVKKNGTCCIADLGLAVRHD |
| | | SATDTIDIAPNHRVGTKRYMAPEVLDDSINMKHFESFKRADYAMGLI |
| | | VFWEIARRCSIGGIHEDYQLPYYDLVPSDPSVEEMRKVVCEQKLRPN |
| | | IPNRWQSCEALRVMAKIMRECWYANGAARLTALRIKKTLSQLSQQEG |
| | | IKM |
| SEQ ID | Human | LQCFCHLCTKDNFTCVTDGLCFVSVTETTDKVIHNSMCIAEIDLIPR |
| NO: 122 | TGFBR1 | DRPFVCAPSSKTGSVTTTYCCNQDHCNKIELPTTGLPLLVQRTIART |
| | isoform 3 | IVLQESIGKGRFGEVWRGKWRGEEVAVKIFSSREERSWFREAEIYQT |
| | (P36897-3) | VMLRHENILGFIAADNKDNGTWTQLWLVSDYHEHGSLFDYLNRYTVT |
| | | VEGMIKLALSTASGLAHLHMEIVGTQGKPAIAHRDLKSKNILVKKNG |
| | | TCCIADLGLAVRHDSATDTIDIAPNHRVGTKRYMAPEVLDDSINMKH |
| | | FESFKRADIYAMGLVFWEIARRCSIGGIHEDYQLPYYDLVPSDPSVE |
| | | EMRKVVCEQKLRPNIPNRWQSCEALRVMAKIMRECWYANGAARLTAL |
| | | RIKKTLSQLSQQEGIKM |
| SEQ ID | Human | LQCFCHLCTKDNFTCVTDGLCFVSVTETTDKVIHNSMCIAEIDLIPR |
| NO: 104 | TGFBR1 | DRPFVCAPSSKTGSVTTTYCCNQDHCNKIELPTTVKSSPGLGPVEL |
| | fragment 1 | |
| SEQ ID | Human | ALQCFCHLCTKDNFTCVTDGLCFVSVTETTDKVIHNSMCIAEIDLIP |
| NO: 105 | TGFB R1 | RDRPFVCAPSSKTGSVTTTYCCNQDHCNKIEL |
| | fragment 2 | |
| SEQ ID | Immature | MGRGLLRGLWPLHIVLWTRIASTIPPHVQKSVNNDMIVTDNNGAVKF |
| NO: 98 | human | PQLCKFCDVRFSTCDNQKSCMSNCSITSICEKPQEVCVAVWRKNDEN |
| | TGFBR2 | ITLETVCHDPKLPYHDFILEDAASPKCIMKEKKKPGETFFMCSCSSD |
| | isoform B | ECNDNIIFSEEYNTSNPDLLLVIFQVTGISLLPPLGVAISVIIIFYC |
| | (short | YRVNRQQKLSSTWETGKTRKLMEFSEHCAIILEDDRSDISSTCANNI |
| | isoform) | NHNTELLPIELDTLVGKGRFAEVYKAKLKQNTSEQFETVAVKIFPYE |
| | (P37173-1) | EYASWKTEKDIFSDINLKHENILQFLTAEERKTELGKQYWLITAFHA |
| | | KGNLQEYLTRHVISWEDLRKLGSSLARGIAHLHSDHTPCGRPKMPIV |
| | | HRDLKSSNILVKNDLTCCLCDFGLSLRLDPTLSVDDLANSGQVGTAR |
| | | YMAPEVLESRMNLENVESFKQTDVYSMALVLWEMTSRCNAVGEVKDY |
| | | EPPFGSKVREHPCVESMKDNVLRDRGRPEIPSFWLNHQGIQMVCETL |
| | | TECWDHDPEARLTAQCVAERFSELEHLDRLSGRSCSEEKIPEDGSLN |
| | | TTK |
| SEQ ID | Human | TIPPHVQKSVNNDMIVTDNNGAVKFPQLCKFCDVRFSTCDNQKSCMS |
| NO: 123 | TGFBR2 | NCSITSICEKPQEVCVAVWRKNDENITLETVCHDPKLPYHDFILEDA |
| | isoform B | ASPKCIMKEKKKPGETFFMCSCSSDECNDNIIFSEEYNTSNPDLLLV |
| | (short | IFQVTGISLLPPLGVAISVIIIFYCYRVNRQQKLSSTWETGKTRKLM |
| | isoform) | EFSEHCAIILEDDRSDISSTCANNINHNTELLPIELDTLVGKGRFAE |
| | (P37173-1) | VYKAKLKQNTSEQFETVAVKIFPYEEYASWKTEKDIFSDINLKHENI |
| | | LQFLTAEERKTELGKQYWLITAFHAKGNLQEYLTRHVISWEDLRKLG |
| | | SSLARGIAHLHSDHTPCGRPKMPIVHRDLKSSNILVKNDLTCCLCDF |
| | | GLSLRLDPTLSVDDLANSGQVGTARYMAPEVLESRMNLENVESFKQT |
| | | DVYSMALVLWEMTSRCNAVGEVKDYEPPFGSKVREHPCVESMKDNVL |
| | | RDRGRPEIPSFWLNHQGIQMVCETLTECWDHDPEARLTAQCVAERFS |
| | | ELEHLDRLSGRSCSEEKIPEDGSLNTTK |
| SEQ ID | Immature | MGRGLLRGLWPLHIVLWTRIASTIPPHVQKSDVEMEAQKDEIICPSC |
| NO: 99 | human | NRTAHPLRHINNDMIVTDNNGAVKFPQLCKFCDVRFSTCDNQKSCMS |
| | TGFBR2 | NCSITSICEKPQEVCVAVWRKNDENITLETVCHDPKLPYHDFILEDA |
| | isoform A | ASPKCIMKEKKKPGETFFMCSCSSDECNDNIIFSEEYNTSNPDLLLV |
| | (long | IFQVTGISLLPPLGVAISVIIIFYCYRVNRQQKLSSTWETGKTRKLM |
| | isoform) | EFSEHCAIILEDDRSDISSTCANNINHNTELLPIELDTLVGKGRFAE |
| | (P37173-2) | VYKAKLKQNTSEQFETVAVKIFPYEEYASWKTEKDIFSDINLKHENI |
| | | LQFLTAEERKTELGKQYWLITAFHAKGNLQEYLTRHVISWEDLRKLG |
| | | SSLARGIAHLHSDHTPCGRPKMPIVHRDLKSSNILVKNDLTCCLCDF |
| | | GLSLRLDPTLSVDDLANSGQVGTARYMAPEVLESRMNLENVESFKQT |
| | | DVYSMALVLWEMTSRCNAVGEVKDYEPPFGSKVREHPCVESMKDNVL |
| | | RDRGRPEIPSFWLNHQGIQMVCETLTECWDHDPEARLTAQCVAERFS |
| | | ELEHLDRLSGRSCSEEKIPEDGSLNTTK |
| SEQ ID | Human | TIPPHVQKSDVEMEAQKDEIICPSCNRTAHPLRHINNDMIVTDNNGA |
| NO: 124 | TGFBR2 | VKFPQLCKFCDVRFSTCDNQKSCMSNCSITSICEKPQEVCVAVWRKN |
| | isoform A | DENITLETVCHDPKLPYHDFILEDAASPKCIMKEKKKPGETFFMCSC |
| | (long | SSDECNDNIIFSEEYNTSNPDLLLVIFQVTGISLLPPLGVAISVIII |
| | isoform) | FYCYRVNRQQKLSSTWETGKTRKLMEFSEHCAIILEDDRSDISSTCA |
| | (P37173-2) | NNINHNTELLPIELDTLVGKGRFAEVYKAKLKQNTSEQFETVAVKIF |
| | | PYEEYASWKTEKDIFSDINLKHENILQFLTAEERKTELGKQYWLITA |
| | | FHAKGNLQEYLTRHVISWEDLRKLGSSLARGIAHLHSDHTPCGRPKM |
| | | PIVHRDLKSSNILVKNDLTCCLCDFGLSLRLDPTLSVDDLANSGQVG |
| | | TARYMAPEVLESRMNLENVESFKQTDVYSMALVLWEMTSRCNAVGEV |
| | | KDYEPPFGSKVREHPCVESMKDNVLRDRGRPEIPSFWLNHQGIQMVC |
| | | ETLTECWDHDPEARLTAQCVAERFSELEHLDRLSGRSCSEEKIPEDG |
| | | SLNTTK |
| SEQ ID | Human | TIPPHVQKSVNNDMIVTDNNGAVKFPQLCKFCDVRFSTCDNQKSCMS |
| NO: 100 | TGFBR2 | NCSITSICEKPQEVCVAVWRKNDENITLETVCHDPKLPYHDFILEDA |
| | fragment 1 | ASPKCIMKEKKKPGETFFMCSCSSDECNDNIIFSEEYNTSNPD |
| | (ECD of | |
| | human | |
| | TGFBR2 | |
| | isoform B) | |
| SEQ ID | Human | IPPHVQKSVNNDMIVTDNNGAVKFPQLCKFCDVRFSTCDNQKSCMSN |
| NO: 101 | TGFBR2 | CSITSICEKPQEVCVAVWRKNDENITLETVCHDPKLPYHDFILEDAA |
| | fragment 2 | SPKCIMKEKKKPGETFFMCSCSSDECNDNIIFSEEYNTSNPD |
| SEQ ID | Human | TIPPHVQKSDVEMEAQKDEIICPSCNRTAHPLRHINNDMIVTDNNGA |
| NO: 102 | TGFBR2 | VKFPQLCKFCDVRFSTCDNQKSCMSNCSITSICEKPQEVCVAVWRKN |
| | fragment 3 | DENITLETVCHDPKLPYHDFILEDAASPKCIMKEKKKPGETFFMCSC |
| | (ECD of | SSDECNDNIIFSEEYNTSNPD |
| | human | |
| | TGFBR2 | |
| | isoform A) | |
| SEQ ID | Human | QLCKFCDVRFSTCDNQKSCMSNCSITSICEKPQEVCVAVWRKNDENI |
| NO: 103 | TGFBR2 | TLETVCHDPKLPYHDFILEDAASPKCIMKEKKKPGETFFMCSCSSDE |
| | fragment 4 | CNDNIIF |
| SEQ ID | Immature | MTSHYVIAIFALMSSCLATAGPEPGALCELSPVSASHPVQALMESFT |
| NO: 106 | human | VLSGCASRGTTGLPQEVHVLNLRTAGQGPGQLQREVTLHLNPISSVH |
| | TGFBR3 | IHHKSVVFLLNSPHPLVWHLKTERLATGVSRLFLVSEGSVVQFSSAN |
| | isoform 1 | FSLTAETEERNFPHGNEHLLNWARKEYGAVTSFTELKIARNIYIKVG |
| | (Q03167-1) | EDQVFPPKCNIGKNFLSLNYLAEYLQPKAAEGCVMSSQPQNEEVHII |
| | | ELITPNSNPYSAFQVDITIDIRPSQEDLEVVKNLILILKCKKSVNWV |
| | | IKSFDVKGSLKIIAPNSIGFGKESERSMTMTKSIRDDIPSTQGNLVK |
| | | WALDNGYSPITSYTMAPVANRFHLRLENNAEEMGDEEVHTIPPELRI |
| | | LLDPGALPALQNPPIRGGEGQNGGLPFPFPDISRRVWNEEGEDGLPR |
| | | PKDPVIPSIQLFPGLREPEEVQGSVDIALSVKCDNEKMIVAVEKDSF |
| | | QASGYSGMDVTLLDPTCKAKMNGTHFVLESPLNGCGTRPRWSALDGV |
| | | VYYNSIVIQVPALGDSSGWPDGYEDLESGDNGFPGDMDEGDASLFTR |
| | | PEIVVFNCSLQQVRNPSSFQEQPHGNITFNMELYNTDLFLVPSQGVF |
| | | SVPENGHVYVEVSVTKAEQELGFAIQTCFISPYSNPDRMSHYTIIEN |
| | | ICPKDESVKFYSPKRVHFPIPQADMDKKRFSFVFKPVFNTSLLFLQC |
| | | ELTLCTKMEKHPQKLPKCVPPDEACTSLDASIIWAMMQNKKTFTKPL |
| | | AVIHHEAESKEKGPSMKEPNPISPPIFHGLDTLTVMGIAFAAFVIGA |
| | | LLTGALWYIYSHTGETAGRQQVPTSPPASENSSAAHSIGSTQSTPCS |
| | | SSSTA |
| SEQ ID | Human | GPEPGALCELSPVSASHPVQALMESFTVLSGCASRGTTGLPQEVHVL |
| NO: 125 | TGFBR3 | NLRTAGQGPGQLQREVTLHLNPISSVHIHHKSVVFLLNSPHPLVWHL |
| | isoform 1 | KTERLATGVSRLFLVSEGSVVQFSSANFSLTAETEERNFPHGNEHLL |
| | (Q03167-1) | NWARKEYGAVTSFTELKIARNIYIKVGEDQVFPPKCNIGKNFLSLNY |
| | | LAEYLQPKAAEGCVMSSQPQNEEVHIIELITPNSNPYSAFQVDITID |
| | | IRPSQEDLEVVKNLILILKCKKSVNWVIKSFDVKGSLKIIAPNSIGF |
| | | GKESERSMTMTKSIRDDIPSTQGNLVKWALDNGYSPITSYTMAPVAN |
| | | RFHLRLENNAEEMGDEEVHTIPPELRILLDPGALPALQNPPIRGGEG |
| | | QNGGLPFPFPDISRRVWNEEGEDGLPRPKDPVIPSIQLFPGLREPEE |
| | | VQGSVDIALSVKCDNEKMIVAVEKDSFQASGYSGMDVTLLDPTCKAK |
| | | MNGTHFVLESPLNGCGTRPRWSALDGVVYYNSIVIQVPALGDSSGWP |
| | | DGYEDLESGDNGFPGDMDEGDASLFTRPEIVVFNCSLQQVRNPSSFQ |
| | | EQPHGNITFNMELYNTDLFLVPSQGVFSVPENGHVYVEVSVTKAEQE |
| | | LGFAIQTCFISPYSNPDRMSHYTIIENICPKDESVKFYSPKRVHFPI |
| | | PQADMDKKRFSFVFKPVFNTSLLFLQCELTLCTKMEKHPQKLPKCVP |
| | | PDEACTSLDASIIWAMMQNKKTFTKPLAVIHHEAESKEKGPSMKEPN |
| | | PISPPIFHGLDTLTVMGIAFAAFVIGALLTGALWYIYSHTGETAGRQ |
| | | QVPTSPPASENSSAAHSIGSTQSTPCSSSSTA |
| SEQ ID | Immature | MTSHYVIAIFALMSSCLATAGPEPGALCELSPVSASHPVQALMESFT |
| NO: 107 | human | VLSGCASRGTTGLPQEVHVLNLRTAGQGPGQLQREVTLHLNPISSVH |
| | TGFBR3 | IHHKSVVFLLNSPHPLVWHLKTERLATGVSRLFLVSEGSVVQFSSAN |
| | isoform 2 | FSLTAETEERNFPHGNEHLLNWARKEYGAVTSFTELKIARNIYIKVG |
| | (Q03167-2) | EDQVFPPKCNIGKNFLSLNYLAEYLQPKAAEGCVMSSQPQNEEVHII |
| | | ELITPNSNPYSAFQVDITIDIRPSQEDLEVVKNLILILKCKKSVNWV |
| | | IKSFDVKGSLKIIAPNSIGFGKESERSMTMTKSIRDDIPSTQGNLVK |
| | | WALDNGYSPITSYTMAPVANRFHLRLENNEEMGDEEVHTIPPELRIL |
| | | LDPGALPALQNPPIRGGEGQNGGLPFPFPDISRRVWNEEGEDGLPRP |
| | | KDPVIPSIQLFPGLREPEEVQGSVDIALSVKCDNEKMIVAVEKDSFQ |
| | | ASGYSGMDVTLLDPTCKAKMNGTHFVLESPLNGCGTRPRWSALDGVV |
| | | YYNSIVIQVPALGDSSGWPDGYEDLESGDNGFPGDMDEGDASLFTRP |
| | | EIVVFNCSLQQVRNPSSFQEQPHGNITFNMELYNTDLFLVPSQGVFS |
| | | VPENGHVYVEVSVTKAEQELGFAIQTCFISPYSNPDRMSHYTIIENI |
| | | CPKDESVKFYSPKRVHFPIPQADMDKKRFSFVFKPVFNTSLLFLQCE |
| | | LTLCTKMEKHPQKLPKCVPPDEACTSLDASIIWAMMQNKKTFTKPLA |
| | | VIHHEAESKEKGPSMKEPNPISPPIFHGLDTLTVMGIAFAAFVIGAL |
| | | LTGALWYIYSHTGETAGRQQVPTSPPASENSSAAHSIGSTQSTPCSS |
| | | SSTA |
| SEQ ID | Human | GPEPGALCELSPVSASHPVQALMESFTVLSGCASRGTTGLPQEVHVL |
| NO: 126 | TGFBR3 | NLRTAGQGPGQLQREVTLHLNPISSVHIHHKSVVFLLNSPHPLVWHL |
| | isoform 2 | KTERLATGVSRLFLVSEGSVVQFSSANFSLTAETEERNFPHGNEHLL |
| | (Q03167-2) | NWARKEYGAVTSFTELKIARNIYIKVGEDQVFPPKCNIGKNFLSLNY |
| | | LAEYLQPKAAEGCVMSSQPQNEEVHIIELITPNSNPYSAFQVDITID |
| | | IRPSQEDLEVVKNLILILKCKKSVNWVIKSFDVKGSLKIIAPNSIGF |
| | | GKESERSMTMTKSIRDDIPSTQGNLVKWALDNGYSPITSYTMAPVAN |
| | | RFHLRLENNEEMGDEEVHTIPPELRILLDPGALPALQNPPIRGGEGQ |
| | | NGGLPFPFPDISRRVWNEEGEDGLPRPKDPVIPSIQLFPGLREPEEV |
| | | QGSVDIALSVKCDNEKMIVAVEKDSFQASGYSGMDVTLLDPTCKAKM |
| | | NGTHFVLESPLNGCGTRPRWSALDGVVYYNSIVIQVPALGDSSGWPD |
| | | GYEDLESGDNGFPGDMDEGDASLFTRPEIVVFNCSLQQVRNPSSFQE |
| | | QPHGNITFNMELYNTDLFLVPSQGVFSVPENGHVYVEVSVTKAEQEL |
| | | GFAIQTCFISPYSNPDRMSHYTIIENICPKDESVKFYSPKRVHFPIP |
| | | QADMDKKRFSFVFKPVFNTSLLFLQCELTLCTKMEKHPQKLPKCVPP |
| | | DEACTSLDASIIWAMMQNKKTFTKPLAVIHHEAESKEKGPSMKEPNP |
| | | ISPPIFHGLDTLTVMGIAFAAFVIGALLTGALWYIYSHTGETAGRQQ |
| | | VPTSPPASENSSAAHSIGSTQSTPCSSSSTA |
| SEQ ID | Human | GPEPGALCELSPVSASHPVQALMESFTVLSGCASRGTTGLPQEVHVL |
| NO: 108 | TGFBR3 | NLRTAGQGPGQLQREVTLHLNPISSVHIHHKSVVFLLNSPHPLVWHL |
| | fragment 1 | KTERLATGVSRLFLVSEGSVVQFSSANFSLTAETEERNFPHGNEHLL |
| | | NWARKEYGAVTSFTELKIARNIYIKVGEDQVFPPKCNIGKNFLSLNY |
| | | LAEYLQPKAAEGCVMSSQPQNEEVHIIELITPNSNPYSAFQVDITID |
| | | IRPSQEDLEVVKNLILILKCKKSVNWVIKSFDVKGSLKIIAPNSIGF |
| | | GKESERSMTMTKSIRDDIPSTQGNLVKWALDNGYSPITSYTMAPVAN |
| | | RFHLRLENNAEEMGDEEVHTIPPELRILLDPGALPALQNPPIRGGEG |
| | | QNGGLPFPFPDISRRVWNEEGEDGLPRPKDPVIPSIQLFPGLREPEE |
| | | VQGSVDIALSVKCDNEKMIVAVEKDSFQASGYSGMDVTLLDPTCKAK |
| | | MNGTHFVLESPLNGCGTRPRWSALDGVVYYNSIVIQVPALGDSSGWP |
| | | DGYEDLESGDNGFPGDMDEGDASLFTRPEIVVFNCSLQQVRNPSSFQ |
| | | EQPHGNITFNMELYNTDLFLVPSQGVFSVPENGHVYVEVSVTKAEQE |
| | | LGFAIQTCFISPYSNPDRMSHYTIIENICPKDESVKFYSPKRVHFPI |
| | | PQADMDKKRFSFVFKPVFNTSLLFLQCELTLCTKMEKHPQKLPKCVP |
| | | PDEACTSLDASIIWAMMQNKKTFTKPLAVIHHEAESKEKGPSMKEPN |
| | | PISPPIFHGLDTLTV |
| SEQ ID | hCH1- | ASTKGPSVFPLAPSSKSTSGGTAALGCLVKDYFPEPVTVSWNSGALT |
| NO: 192 | hFc_Hole- | SGVHTFPAVLQSSGLYSLSSVVTVPSSSLGTQTYICNVNHKPSNTKV |
| | 3x4GS- | DKRVEPKSCDKTHTCPPCPAPELLGGPSVFLFPPKPKDTLMISRTPE |
| | TGFbR2 | VTCVVVDVSHEDPEVKFNWYVDGVEVHNAKTKPREEQYNSTYRVVSV |
| | | LTVLHQDWLNGKEYKCKVSNKALPAPIEKTISKAKGQPREPQVCTLP |
| | | PSREEMTKNQVSLSCAVKGFYPSDIAVEWESNGQPENNYKTTPPVLD |
| | | SDGSFFLVSKLTVDKSRWQQGNVFSCSVMHEALHNHYTQKSLSLSPG |
| | | XGGGGSGGGGSGGGGSIPPHVQKSVNNDMIVTDNNGAVKFPQLCKFC |
| | | DVRFSTCDNQKSCMSNCSITSICEKPQEVCVAVWRKNDENITLETVC |
| | | HDPKLPYHDFILEDAASPKCIMKEKKKPGETFFMCSCSSDECNDNII |
| | | FSEEYNTSNPD, wherein X is Korabsent |
| SEQ ID | hCH1- | ASTKGPSVFPLAPSSKSTSGGTAALGCLVKDYFPEPVTVSWNSGALT |
| NO: 193 | hFc_Knob- | SGVHTFPAVLQSSGLYSLSSVVTVPSSSLGTQTYICNVNHKPSNTKV |
| | 3x4GS- | DKRVEPKSCDKTHTCPPCPAPELLGGPSVFLFPPKPKDTLMISRTPE |
| | TGFbR2 | VTCVVVDVSHEDPEVKFNWYVDGVEVHNAKTKPREEQYNSTYRVVSV |
| | | LTVLHQDWLNGKEYKCKVSNKALPAPIEKTISKAKGQPREPQVYTLP |
| | | PCREEMTKNQVSLWCLVKGFYPSDIAVEWESNGQPENNYKTTPPVLD |
| | | SDGSFFLYSKLTVDKSRWQQGNVFSCSVMHEALHNHYTQKSLSLSPG |
| | | XGGGGSGGGGSGGGGSIPPHVQKSVNNDMIVTDNNGAVKFPQLCKFC |
| | | DVRFSTCDNQKSCMSNCSITSICEKPQEVCVAVWRKNDENITLETVC |
| | | HDPKLPYHDFILEDAASPKCIMKEKKKPGETFFMCSCSSDECNDNII |
| | | FSEEYNTSNPD, wherein X is Korabsent |
| SEQ ID | hFc_Hole- | DKTHTCPPCPAPELLGGPSVFLFPPKPKDTLMISRTPEVTCVVVDVS |
| NO: 194 | 3x4GS- | HEDPEVKFNWYVDGVEVHNAKTKPREEQYNSTYRVVSVLTVLHQDWL |
| | TGFbR2 | NGKEYKCKVSNKALPAPIEKTISKAKGQPREPQVCTLPPSREEMTKN |
| | | QVSLSCAVKGFYPSDIAVEWESNGQPENNYKTTPPVLDSDGSFFLVS |
| | | KLTVDKSRWQQGNVFSCSVMHEALHNHYTQKSLSLSPGXGGGGSGGG |
| | | GSGGGGSIPPHVQKSVNNDMIVTDNNGAVKFPQLCKFCDVRFSTCDN |
| | | QKSCMSNCSITSICEKPQEVCVAVWRKNDENITLETVCHDPKLPYHD |
| | | FILEDAASPKCIMKEKKKPGETFFMCSCSSDECNDNIIFSEEYNTSN |
| | | PD, wherein X is Korabsent |
| SEQ ID | hFc_Knob- | DKTHTCPPCPAPELLGGPSVFLFPPKPKDTLMISRTPEVTCVVVDVS |
| | 3x4GS- | HEDPEVKFNWYVDGVEVHNAKTKPREEQYNSTYRVVSVLTVLHQDWL |
| NO: 195 | TGFbR2 | NGKEYKCKVSNKALPAPIEKTISKAKGQPREPQVYTLPPCREEMTKN |
| | | QVSLWCLVKGFYPSDIAVEWESNGQPENNYKTTPPVLDSDGSFFLYS |
| | | KLTVDKSRWQQGNVFSCSVMHEALHNHYTQKSLSLSPGXGGGGSGGG |
| | | GSGGGGSIPPHVQKSVNNDMIVTDNNGAVKFPQLCKFCDVRFSTCDN |
| | | QKSCMSNCSITSICEKPQEVCVAVWRKNDENITLETVCHDPKLPYHD |
| | | FILEDAASPKCIMKEKKKPGETFFMCSCSSDECNDNIIFSEEYNTSN |
| | | PD, wherein X is Korabsent |
| SEQ ID | TGFbR2- | IPPHVQKSVNNDMIVTDNNGAVKFPQLCKFCDVRFSTCDNQKSCMSN |
| NO: 196 | 3x4GS- | CSITSICEKPQEVCVAVWRKNDENITLETVCHDPKLPYHDFILEDAA |
| | hCH1- | SPKCIMKEKKKPGETFFMCSCSSDECNDNIIFSEEYNTSNPDGGGGS |
| | hFc_Hole | GGGGSGGGGSASTKGPSVFPLAPSSKSTSGGTAALGCLVKDYFPEPV |
| | | TVSWNSGALTSGVHTFPAVLQSSGLYSLSSVVTVPSSSLGTQTYICN |
| | | VNHKPSNTKVDKRVEPKSCDKTHTCPPCPAPELLGGPSVFLFPPKPK |
| | | DTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQ |
| | | PREPQVCTLPPSREEMTKNQVSLSCAVKGFYPSDIAVEWESNGQPEN |
| | | NYKTTPPVLDSDGSFFLVSKLTVDKSRWQQGNVFSCSVMHEALHNHY |
| | | TQKSLSLSPGX, wherein X is Korabsent |
| SEQ ID | TGFbR2- | IPPHVQKSVNNDMIVTDNNGAVKFPQLCKFCDVRFSTCDNQKSCMSN |
| NO: 197 | 3x4GS- | CSITSICEKPQEVCVAVWRKNDENITLETVCHDPKLPYHDFILEDAA |
| | hCH1- | SPKCIMKEKKKPGETFFMCSCSSDECNDNIIFSEEYNTSNPDGGGGS |
| | hFc_Knob | GGGGSGGGGSASTKGPSVFPLAPSSKSTSGGTAALGCLVKDYFPEPV |
| | | TVSWNSGALTSGVHTFPAVLQSSGLYSLSSVVTVPSSSLGTQTYICN |
| | | VNHKPSNTKVDKRVEPKSCDKTHTCPPCPAPELLGGPSVFLFPPKPK |
| | | DTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQ |
| | | PREPQVYTLPPCREEMTKNQVSLWCLVKGFYPSDIAVEWESNGQPEN |
| | | NYKTTPPVLDSDGSFFLYSKLTVDKSRWQQGNVFSCSVMHEALHNHY |
| | | TQKSLSLSPGX, wherein X is Korabsent |
| SEQ ID | TGFbR2- | IPPHVQKSVNNDMIVTDNNGAVKFPQLCKFCDVRFSTCDNQKSCMSN |
| NO: 198 | 3x4GS- | CSITSICEKPQEVCVAVWRKNDENITLETVCHDPKLPYHDFILEDAA |
| | hCLIg_vl | SPKCIMKEKKKPGETFFMCSCSSDECNDNIIFSEEYNTSNPDGGGGS |
| | | GGGGSGGGGSGQPKANPTVTLFPPSSEELQANKATLVCLISDFYPGA |
| | | VTVAWKADGSPVKAGVETTKPSKQSNNKYAASSYLSLTPEQWKSHRS |
| | | YSCQVTHEGSTVEKTVAPTECS |
| SEQ ID | TGFI3R2- | IPPHVQKSVNNDMIVTDNNGAVKFPQLCKFCDVRFSTCDNQKSCMSN |
| NO: 199 | 3x4G5- | CSITSICEKPQEVCVAVWRKNDENITLETVCHDPKLPYHDFILEDAA |
| | hCLIg_vk | SPKCIMKEKKKPGETFFMCSCSSDECNDNIIFSEEYNTSNPDGGGGS |
| | | GGGGSGGGGSRTVAAPSVFIFPPSDEQLKSGTASVVCLLNNFYPREA |
| | | KVQWKVDNALQSGNSQESVTEQDSKDSTYSLSSTLTLSKADYEKHKV |
| | | YACEVTHQGLSSPVTKSFNRGEC |

### Cytokine Molecules

Cytokines are generally polypeptides that influence cellular activity, for example, through signal transduction pathways. Accordingly, a cytokine of the multispecific or multifunctional polypeptide is useful and can be associated with receptor-mediated signaling that transmits a signal from outside the cell membrane to modulate a response within the cell. Cytokines are proteinaceous signaling compounds that are mediators of the immune response. They control many different cellular functions including proliferation, differentiation and cell survival/apoptosis; cytokines are also involved in several pathophysiological processes including viral infections and autoimmune diseases. Cytokines are synthesized under various stimuli by a variety of cells of both the innate (monocytes, macrophages, dendritic cells) and adaptive (T- and B-cells) immune systems. Cytokines can be classified into two groups: pro- and anti-inflammatory. Pro-inflammatory cytokines, including IFNγ, IL-1, IL-6 and TNF-alpha, are predominantly derived from the innate immune cells and Th1 cells. Anti-inflammatory cytokines, including IL-10, IL-4, IL-13 and IL-5, are synthesized from Th2 immune cells.

The present disclosure provides, inter alia, multispecific (e.g., bi-, tri-, quad-specific) or multifunctional molecules, that include, e.g., are engineered to contain, one or more cytokine molecules, e.g., immunomodulatory (e.g., proinflammatory) cytokines and variants, e.g., functional variants, thereof. Accordingly, in some embodiments, the cytokine molecule is an interleukin or a variant, e.g., a functional variant thereof. In some embodiments the interleukin is a proinflammatory interleukin. In some embodiments the interleukin is chosen from interleukin-2 (IL-2), interleukin-12 (IL-12), interleukin-15 (IL-15), interleukin-18 (IL-18), interleukin-21 (IL-21), interleukin-7 (IL-7), or interferon gamma. In some embodiments, the cytokine molecule is a proinflammatory cytokine.

In certain embodiments, the cytokine is a single chain cytokine. In certain embodiments, the cytokine is a multichain cytokine (e.g., the cytokine comprises 2 or more (e.g., 2) polypeptide chains. An exemplary multichain cytokine is IL-12.

Examples of useful cytokines include, but are not limited to, GM-CSF, IL-1α, IL-1β, IL-2, IL-3, IL-4, IL-5, IL-6, IL-7, IL-8, IL-10, IL-12, IL-21, IFN-α, IFN-β, IFN-γ, MIP-1α, MIP-1β, TGF-β, TNF-α, and TNFβ. In one embodiment the cytokine of the multispecific or multifunctional polypeptide is a cytokine selected from the group of GM-CSF, IL-2, IL-7, IL-8, IL-10, IL-12, IL-15, IL-21, IFN-α, IFN-γ, MIP-1α, MIP-1β and TGF-β. In one embodiment the cytokine of the i the multispecific or multifunctional polypeptide is a cytokine selected from the group of IL-2, IL-7, IL-10, IL-12, IL-15, IFN-α, and IFN-γ. In certain embodiments the cytokine is mutated to remove N- and/or O-glycosylation sites. Elimination of glycosylation increases homogeneity of the product obtainable in recombinant production.

In one embodiment, the cytokine of the multispecific or multifunctional polypeptide is IL-2. In a specific embodiment, the IL-2 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity. In another particular embodiment the IL-2 cytokine is a mutant IL-2 cytokine having reduced binding affinity to the .alpha.-subunit of the IL-2 receptor. Together with the .beta.- and .gamma.-subunits (also known as CD122 and CD132, respectively), the .alpha.-subunit (also known as CD25) forms the heterotrimeric high-affinity IL-2 receptor, while the dimeric receptor consisting only of the β- and γ-subunits is termed the intermediate-affinity IL-2 receptor. As described in PCT patent application number PCT/EP2012/051991, which is incorporated herein by reference in its entirety, a mutant IL-2 polypeptide with reduced binding to the .alpha.-subunit of the IL-2 receptor has a reduced ability to induce IL-2 signaling in regulatory T cells, induces less activation-induced cell death (AICD) in T cells, and has a reduced toxicity profile in vivo, compared to a wild-type IL-2 polypeptide. The use of such an cytokine with reduced toxicity is particularly advantageous in a multispecific or multifunctional polypeptide according to the invention, having a long serum half-life due to the presence of an Fc domain. In one embodiment, the mutant IL-2 cytokine of the multispecific or multifunctional polypeptide according to the invention comprises at least one amino acid mutation that reduces or abolishes the affinity of the mutant IL-2 cytokine to the .alpha.-subunit of the IL-2 receptor (CD25) but preserves the affinity of the mutant IL-2 cytokine to the intermediate-affinity IL-2 receptor (consisting of the β and γ subunits of the IL-2 receptor), compared to the non-mutated IL-2 cytokine. In one embodiment the one or more amino acid mutations are amino acid substitutions. In a specific embodiment, the mutant IL-2 cytokine comprises one, two or three amino acid substitutions at one, two or three position(s) selected from the positions corresponding to residue 42, 45, and 72 of human IL-2. In a more specific embodiment, the mutant IL-2 cytokine comprises three amino acid substitutions at the positions corresponding to residue 42, 45 and 72 of human IL-2. In an even more specific embodiment, the mutant IL-2 cytokine is human IL-2 comprising the amino acid substitutions F42A, Y45A and L72G. In one embodiment the mutant IL-2 cytokine additionally comprises an amino acid mutation at a position corresponding to position 3 of human IL-2, which eliminates the O-glycosylation site of IL-2. Particularly, said additional amino acid mutation is an amino acid substitution replacing a threonine residue by an alanine residue. A particular mutant IL-2 cytokine useful in the invention comprises four amino acid substitutions at positions corresponding to residues 3, 42, 45 and 72 of human IL-2. Specific amino acid substitutions are T3A, F42A, Y45A and L72G. As demonstrated in PCT patent application number PCT/EP2012/051991 and in the appended Examples, said quadruple mutant IL-2 polypeptide (IL-2 qm) exhibits no detectable binding to CD25, reduced ability to induce apoptosis in T cells, reduced ability to induce IL-2 signaling in T.sub.reg cells, and a reduced toxicity profile in vivo. However, it retains ability to activate IL-2 signaling in effector cells, to induce proliferation of effector cells, and to generate IFN-γ as a secondary cytokine by NK cells.

The IL-2 or mutant IL-2 cytokine according to any of the above embodiments may comprise additional mutations that provide further advantages such as increased expression or stability. For example, the cysteine at position 125 may be replaced with a neutral amino acid such as alanine, to avoid the formation of disulfide-bridged IL-2 dimers. Thus, in certain embodiments the IL-2 or mutant IL-2 cytokine of the multispecific or multifunctional polypeptide according to the invention comprises an additional amino acid mutation at a position corresponding to residue 125 of human IL-2. In one embodiment said additional amino acid mutation is the amino acid substitution C125A.

In a specific embodiment the IL-2 cytokine of the multispecific or multifunctional polypeptide comprises the polypeptide sequence of SEQ ID NO: 37 [APTSSSTKKTQLQLEHLLLDLQMILNGINNYKNPKLTRMLTFKFYMPKKATELKHLQCL EEELKPLEEVLNLAQSKNFHLRPRDLISNINVIVLELKGSETTFMCEYADETATIVEFLNR WITFAQSIISTLT]. In another specific embodiment the IL-2 cytokine of the multispecific or multifunctional polypeptide comprises the polypeptide sequence of SEQ ID NO: 38 [APASSSTKKTQLQLEHLLLDLQMILNGINNYKNPKLTRMLTAKFAMPKKATELKHLQC LEEELKPLEEVLNGAQSKNFHLRPRDLISNINVIVLELKGSETTFMCEYADETATIVEFLN RWITFAQSIISTLT].

In another embodiment the cytokine of the multispecific or multifunctional polypeptide is IL-12. In a specific embodiment said IL-12 cytokine is a single chain IL-12 cytokine. In an even more specific embodiment the single chain IL-12 cytokine comprises the polypeptide sequence of SEQ ID NO: 39 [IWELKKDVYVVELDWYPDAPGEMVVLTCDTPEEDGITWTLDQSSEVLGSGKTLTIQVK EFGDAGQYTCHKGGEVLSHSLLLLHKKEDGIWSTDILKDQKEPKNKTFLRCEAKNYSGR FTCWWLTTISTDLTFSVKSSRGSSDPQGVTCGAATLSAERVRGDNKEYEYSVECQEDSA CPAAEESLPIEVMVDAVHKLKYENYTSSFFIRDIIKPDPPKNLQLKPLKNSRQVEVSWEY PDTWSTPHSYFSLTFCVQVQGKSKREKKDRVFTDKTSATVICRKNASISVRAQDRYYSS SWSEWASVPCSGGGGSGGGGSGGGGSRNLPVATPDPGMFPCLHHSQNLLRAVSNMLQ KARQTLEFYPCTSEEIDHEDITKDKTSTVEACLPLELTKNESCLNSRETSFITNGSCLASRK TSFMMALCLSSIYEDLKMYQVEFKTMNAKLLMDPKRQIFLDQNMLAVIDELMQALNFN SETVPQKSSLEEPDFYKTKIKLCILLHAFRIRAVTIDRVMSYLNAS]. In one embodiment, the IL-12 cytokine can elicit one or more of the cellular responses selected from the group consisting of: proliferation in a NK cell, differentiation in a NK cell, proliferation in a T cell, and differentiation in a T cell.

In another embodiment the cytokine of the multispecific or multifunctional polypeptide is IL-10. In a specific embodiment said IL-10 cytokine is a single chain IL-10 cytokine. In an even more specific embodiment the single chain IL-10 cytokine comprises the polypeptide sequence of SEQ ID NO: 40 [SPGQGTQSENSCTHFPGNLPNMLRDLRDAFSRVKTFFQMKDQLDNLLLKESLLEDFKG YLGCQALSEMIQFYLEEVMPQAENQDPDIKAHVNSLGENLKTLRLRLRRCHRFLPCENK SKAVEQVKNAFNKLQEKGIYKAMSEFDIFINYIEAYMTMKIRNGGGGSGGGGSGGGGS GGGGSSPGQGTQSENSCTHFPGNLPNMLRDLRDAFSRVKTFFQMKDQLDNLLLKESLLE DFKGYLGCQALSEMIQFYLEEVMPQAENQDPDIKAHVNSLGENLKTLRLRLRRCHRFLP CENKSKAVEQVKNAFNKLQEKGIYKAMSEFDIFINYIEAYMTMKIRN]. In another specific embodiment the IL-10 cytokine is a monomeric IL-10 cytokine. In a more specific embodiment the monomeric IL-10 cytokine comprises the polypeptide sequence of SEQ ID NO: 41 [SPGQGTQSENSCTHFPGNLPNMLRDLRDAFSRVKTFFQMKDQLDNLLLKESLLEDFKG YLGCQALSEMIQFYLEEVMPQAENQDPDIKAHVNSLGENLKTLRLRLRRCHRFLPCENG GGSGGKSKAVEQVKNAFNKLQEKGIYKAMSEFDIFINYIEAYMTMKIRN]. In one embodiment, the IL-10 cytokine can elicit one or more of the cellular responses selected from the group consisting of: inhibition of cytokine secretion, inhibition of antigen presentation by antigen presenting cells, reduction of oxygen radical release, and inhibition of T cell proliferation. A multispecific or multifunctional polypeptide according to the invention wherein the cytokine is IL-10 is particularly useful for downregulation of inflammation, e.g. in the treatment of an inflammatory disorder.

In another embodiment, the cytokine of the multispecific or multifunctional polypeptide is IL-15. In a specific embodiment said IL-15 cytokine is a mutant IL-15 cytokine having reduced binding affinity to the α-subunit of the IL-15 receptor. Without wishing to be bound by theory, a mutant IL-15 polypeptide with reduced binding to the .alpha.-subunit of the IL-15 receptor has a reduced ability to bind to fibroblasts throughout the body, resulting in improved pharmacokinetics and toxicity profile, compared to a wild-type IL-15 polypeptide. The use of an cytokine with reduced toxicity, such as the described mutant IL-2 and mutant IL-15 effector moieties, is particularly advantageous in a multispecific or multifunctional polypeptide according to the invention, having a long serum half-life due to the presence of an Fc domain. In one embodiment the mutant IL-15 cytokine of the multispecific or multifunctional polypeptide according to the invention comprises at least one amino acid mutation that reduces or abolishes the affinity of the mutant IL-15 cytokine to the .alpha.-subunit of the IL-15 receptor but preserves the affinity of the mutant IL-15 cytokine to the intermediate-affinity IL-15/IL-2 receptor (consisting of the .beta.- and .gamma.-subunits of the IL-15/IL-2 receptor), compared to the non-mutated IL-15 cytokine. In one embodiment the amino acid mutation is an amino acid substitution. In a specific embodiment, the mutant IL-15 cytokine comprises an amino acid substitution at the position corresponding to residue 53 of human IL-15. In a more specific embodiment, the mutant IL-15 cytokine is human IL-15 comprising the amino acid substitution E53A. In one embodiment the mutant IL-15 cytokine additionally comprises an amino acid mutation at a position corresponding to position 79 of human IL-15, which eliminates the N-glycosylation site of IL-15. Particularly, said additional amino acid mutation is an amino acid substitution replacing an asparagine residue by an alanine residue. In an even more specific embodiment the IL-15 cytokine comprises the polypeptide sequence of SEQ ID NO: 42 [NWVNVISDLKKIEDLIQSMHIDATLYTESDVHPSCKVTAMKCFLLELQVISLASGDASIH DTVENLIILANNSLSSNGAVTESGCKECEELEEKNIKEFLQSFVHIVQMFINTS]. In one embodiment, the IL-15 cytokine can elicit one or more of the cellular responses selected from the group consisting of proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity.

Mutant cytokine molecules useful as effector moieties in the multispecific or multifunctional polypeptide can be prepared by deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing. Substitution or insertion may involve natural as well as non-natural amino acid residues. Amino acid modification includes well known methods of chemical modification such as the addition or removal of glycosylation sites or carbohydrate attachments, and the like.

In one embodiment, the cytokine, particularly a single-chain cytokine, of the multispecific or multifunctional polypeptide is GM-CSF. In a specific embodiment, the GM-CSF cytokine can elicit proliferation and/or differentiation in a granulocyte, a monocyte or a dendritic cell. In one embodiment, the cytokine, particularly a single-chain cytokine, of the multispecific or multifunctional polypeptide is IFN-α. In a specific embodiment, the IFN-α cytokine can elicit one or more of the cellular responses selected from the group consisting of: inhibiting viral replication in a virus-infected cell, and upregulating the expression of major histocompatibility complex I (MHC I). In another specific embodiment, the IFN-α cytokine can inhibit proliferation in a tumor cell. In one embodiment the cytokine, particularly a single-chain cytokine, of the multispecific or multifunctional polypeptide is IFNγ. In a specific embodiment, the IFN-γ cytokine can elicit one or more of the cellular responses selected from the group of: increased macrophage activity, increased expression of MHC molecules, and increased NK cell activity. In one embodiment the cytokine, particularly a single-chain cytokine, of the multispecific or multifunctional polypeptide is IL-7. In a specific embodiment, the IL-7 cytokine can elicit proliferation of T and/or B lymphocytes. In one embodiment, the cytokine, particularly a single-chain cytokine, of the multispecific or multifunctional polypeptide is IL-8. In a specific embodiment, the IL-8 cytokine can elicit chemotaxis in neutrophils. In one embodiment, the cytokine, particularly a single-chain cytokine, of the multispecific or multifunctional polypeptide, is MIP-1α. In a specific embodiment, the MIP-1α cytokine can elicit chemotaxis in monocytes and T lymphocyte cells. In one embodiment, the cytokine, particularly a single-chain cytokine, of the multispecific or multifunctional polypeptide is MIP-10. In a specific embodiment, the MIP-10 cytokine can elicit chemotaxis in monocytes and T lymphocyte cells. In one embodiment, the cytokine, particularly a single-chain cytokine, of the multispecific or multifunctional polypeptide is TGF-β. In a specific embodiment, the TGF-β cytokine can elicit one or more of the cellular responses selected from the group consisting of: chemotaxis in monocytes, chemotaxis in macrophages, upregulation of IL-1 expression in activated macrophages, and upregulation of IgA expression in activated B cells.

In one embodiment, the multispecific or multifunctional polypeptide of the invention binds to an cytokine receptor with a dissociation constant (K_{D}) that is at least about 1, 1.5, 2, 2.5, 3, 3.5, 4, 4.5, 5, 5.5, 6, 6.5, 7, 7.5, 8, 8.5, 9, 9.5 or 10 times greater than that for a control cytokine. In another embodiment, the multispecific or multifunctional polypeptide binds to an cytokine receptor with a K_{D} that is at least 2, 3, 4, 5, 6, 7, 8, 9, or 10 times greater than that for a corresponding multispecific or multifunctional polypeptide comprising two or more effector moieties. In another embodiment, the multispecific or multifunctional polypeptide binds to an cytokine receptor with a dissociation constant K_{D} that is about 10 times greater than that for a corresponding the multispecific or multifunctional polypeptide comprising two or more cytokines.

In some embodiments, the multispecific molecules disclosed herein include a cytokine molecule. In embodiments, the cytokine molecule includes a full length, a fragment or a variant of a cytokine; a cytokine receptor domain, e.g., a cytokine receptor dimerizing domain; or an agonist of a cytokine receptor, e.g., an antibody molecule (e.g., an agonistic antibody) to a cytokine receptor.

In some embodiments the cytokine molecule is chosen from IL-2, IL-12, IL-15, IL-18, IL-7, IL-21, or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain.

In other embodiments, the cytokine molecule is an agonist of a cytokine receptor, e.g., an antibody molecule (e.g., an agonistic antibody) to a cytokine receptor chosen from an IL-15Ra or IL-21R.

In one embodiment, the cytokine molecule is IL-15, e.g., human IL-15 (e.g., comprising the amino acid sequence: NWVNVISDLKKIEDLIQSMHIDATLYTESDVHPSCKVTAMKCFLLELQVISLESGDASIH DTVENLIILANNSLSSNGNVTESGCKECEELEEKNIKEFLQSFVHIVQMFINTS (SEQ ID NO: 43), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 43.

In some embodiments, the cytokine molecule comprises a receptor dimerizing domain, e.g., an IL15Ralpha dimerizing domain. In one embodiment, the IL15Ralpha dimerizing domain comprises the amino acid sequence: MAPRRARGCRTLGLPALLLLLLLRPPATRGITCPPPMSVEHADIWVKSYSLYSRERYICN SGFKRKAGTSSLTECVL (SEQ ID NO: 44), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 44. In some embodiments, the cytokine molecule (e.g., IL-15) and the receptor dimerizing domain (e.g., an IL15Ralpha dimerizing domain) of the multispecific molecule are covalently linked, e.g., via a linker (e.g., a Gly-Ser linker, e.g., a linker comprising the amino acid sequence SGGSGGGGSGGGSGGGGSLQ (SEQ ID NO: 45). In other embodiments, the cytokine molecule (e.g., IL-15) and the receptor dimerizing domain (e.g., an IL15Ralpha dimerizing domain) of the multispecific molecule are not covalently linked, e.g., are non-covalently associated.

In other embodiments, the cytokine molecule is IL-2, e.g., human IL-2 (e.g., comprising the amino acid sequence: APTSSSTKKTQLQLEHLLLDLQMILNGINNYKNPKLTRMLTFKFYMPKKATELKHLQCL EEELKPLEEVLNLAQSKNFHLRPRDLISNINVIVLELKGSETTFMCEYADETATIVEFLNR WITFCQSIISTLT (SEQ ID NO: 46), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO:46).

In other embodiments, the cytokine molecule is IL-18, e.g., human IL-18 (e.g., comprising the amino acid sequence: YFGKLESKLSVIRNLNDQVLFIDQGNRPLFEDMTDSDCRDNAPRTIFIISMYKDSQPRGM AVTISVKCEKISTLSCENKIISFKEMNPPDNIKDTKSDIIFFQRSVPGHDNKMQFESSSY EGYFLACEKERDLFKLILKKEDELGDRSIMFTVQNED (SEQ ID NO: 47), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 47).

In other embodiments, the cytokine molecule is IL-21, e.g., human IL-21 (e.g., comprising the amino acid sequence: QGQDRHMIRMRQLIDIVDQLKNYVNDLVPEFLPAPEDVETNCEWSAFSCFQKAQLKSA NTGNNERIINVSIKKLKRKPPSTNAGRRQKHRLTCPSCDSYEKKPPKEFLERFKSLLQKMI HQHLSSRTHGSEDS (SEQ ID NO: 48), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 48).

In yet other embodiments, the cytokine molecule is interferon gamma, e.g., human interferon gamma (e.g., comprising the amino acid sequence: QDPYVKEAENLKKYFNAGHSDVADNGTLFLGILKNWKEESDRKIMQSQIVSFYFKLFK NFKDDQSIQKSVETIKEDMNVKFFNSNKKKRDDFEKLTNYSVTDLNVQRKAIHELIQVM AELSPAAKTGKRKRSQMLFRG (SEQ ID NO: 49), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 49).

### Immune Cell Engagers

The immune cell engagers of the multispecific or multifunctional molecules disclosed herein can mediate binding to, and/or activation of, an immune cell, e.g., an immune effector cell. In some embodiments, the immune cell is chosen from a T cell, an NK cell, a B cell, a dendritic cell, or a macrophage cell engager, or a combination thereof. In some embodiments, the immune cell engager is chosen from one, two, three, or all of a T cell engager, NK cell engager, a B cell engager, a dendritic cell engager, or a macrophage cell engager, or a combination thereof. The immune cell engager can be an agonist of the immune system. In some embodiments, the immune cell engager can be an antibody molecule, a ligand molecule (e.g., a ligand that further comprises an immunoglobulin constant region, e.g., an Fc region), a small molecule, a nucleotide molecule.

### Natural Killer Cell Engagers

Natural Killer (NK) cells recognize and destroy tumors and virus-infected cells in an antibody-independent manner. The regulation of NK cells is mediated by activating and inhibiting receptors on the NK cell surface. One family of activating receptors is the natural cytotoxicity receptors (NCRs) which include NKp30, NKp44 and NKp46. The NCRs initiate tumor targeting by recognition of heparan sulfate on cancer cells. NKG2D is a receptor that provides both stimulatory and costimulatory innate immune responses on activated killer (NK) cells, leading to cytotoxic activity. DNAM1 is a receptor involved in intercellular adhesion, lymphocyte signaling, cytotoxicity and lymphokine secretion mediated by cytotoxic T-lymphocyte (CTL) and NK cell. DAP10 (also known as HCST) is a transmembrane adapter protein which associates with KLRK1 to form an activation receptor KLRK1-HCST in lymphoid and myeloid cells; this receptor plays a major role in triggering cytotoxicity against target cells expressing cell surface ligands such as MHC class I chain-related MICA and MICB, and U(optionally L1)6-binding proteins (ULBPs); it KLRK1-HCST receptor plays a role in immune surveillance against tumors and is required for cytolysis of tumors cells; indeed, melanoma cells that do not express KLRK1 ligands escape from immune surveillance mediated by NK cells. CD16 is a receptor for the Fc region of IgG, which binds complexed or aggregated IgG and also monomeric IgG and thereby mediates antibody-dependent cellular cytotoxicity (ADCC) and other antibody-dependent responses, such as phagocytosis.

In some embodiments, the NK cell engager is a viral hemagglutinin (HA), HA is a glycoprotein found on the surface of influenza viruses. It is responsible for binding the virus to cells with sialic acid on the membranes, such as cells in the upper respiratory tract or erythrocytes. HA has at least 18 different antigens. These subtypes are named H1 through H18. NCRs can recognize viral proteins. NKp46 has been shown to be able to interact with the HA of influenza and the HA-NA of Paramyxovirus, including Sendai virus and Newcastle disease virus. Besides NKp46, NKp44 can also functionally interact with HA of different influenza subtypes.

The present disclosure provides, inter alia, multispecific (e.g., bi-, tri-, quad-specific) or multifunctional molecules, that are engineered to contain one or more NK cell engagers that mediate binding to and/or activation of an NK cell. Accordingly, in some embodiments, the NK cell engager is selected from an antigen binding domain or ligand that binds to (e.g., activates): NKp30, NKp40, NKp44, NKp46, NKG2D, DNAM1, DAP10, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, or CD160.

In one embodiment, the NK cell engager is a ligand of NKp30 is a B7-6, e.g., comprises the amino acid sequence of: DLKVEMMAGGTQITPLNDNVTIFCNIFYSQPLNITSMGITWFWKSLTFDKEVKVFEFFGD HQEAFRPGAIVSPWRLKSGDASLRLPGIQLEEAGEYRCEVVVTPLKAQGTVQLEVVASP ASRLLLDQVGMKENEDKYMCESSGFYPEAINITWEKQTQKFPHPIEISEDVITGPTIKNM DGTFNVTSCLKLNSSQEDPGTVYQCVVRHASLHTPLRSNFTLTAARHSLSETEKTDNFS (SEQ ID NO: 50), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 50.

In other embodiments, the NK cell engager is a ligand of NKp44 or NKp46, which is a viral HA. Viral hemagglutinins (HA) are glyco proteins which are on the surface of viruses. HA proteins allow viruses to bind to the membrane of cells via sialic acid sugar moieties which contributes to the fusion of viral membranes with the cell membranes (see e.g., Eur J Immunol. 2001 September; 31(9):2680-9 “Recognition of viral hemagglutinins by NKp44 but not by NKp30”; and Nature. 2001 Feb. 22; 409(6823):1055-60 “Recognition of haemagglutinins on virus-infected cells by NKp46 activates lysis by human NK cells” the contents of each of which are incorporated by reference herein).

In other embodiments, the NK cell engager is a ligand of NKG2D chosen from MICA, MICB, or ULBP1, e.g., wherein:
(i) MICA comprises the amino acid sequence: EPHSLRYNLTVLSWDGSVQSGFLTEVHLDGQPFLRCDRQKCRAKPQGQWAEDVLGNK TWDRETRDLTGNGKDLRMTLAHIKDQKEGLHSLQEIRVCEIHEDNSTRSSQHFYYDGEL FLSQNLETKEWTMPQSSRAQTLAMNVRNFLKEDAMKTKTHYHAMHADCLQELRRYLK SGVVLRRTVPPMVNVTRSEASEGNITVTCRASGFYPWNITLSWRQDGVSLSHDTQQWG DVLPDGNGTYQTWVATRICQGEEQRFTCYMEHSGNHSTHPVPSGKVLVLQSHW (SEQ ID NO: 51), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 51;
(ii) MICB comprises the amino acid sequence: AEPHSLRYNLMVLSQDESVQSGFLAEGHLDGQPFLRYDRQKRRAKPQGQWAEDVLGA KTWDTETEDLTENGQDLRRTLTHIKDQKGGLHSLQEIRVCEIHEDSSTRGSRHFYYDGEL FLSQNLETQESTVPQSSRAQTLAMNVTNFWKEDAMKTKTHYRAMQADCLQKLQRYLK SGVAIRRTVPPMVNVTCSEVSEGNITVTCRASSFYPRNITLTWRQDGVSLSHNTQQWGD VLPDGNGTYQTWVATRIRQGEEQRFTCYMEHSGNHGTHPVPSGKVLVLQSQRTD (SEQ ID NO: 52), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 52; or
(iii) ULBP1 comprises the amino acid sequence: GWVDTHCLCYDFIITPKSRPEPQWCEVQGLVDERPFLHYDCVNHKAKAFASLGKKVNV TKTWEEQTETLRDVVDFLKGQLLDIQVENLIPIEPLTLQARMSCEHEAHGHGRGSWQFL FNGQKFLLFDSNNRKWTALHPGAKKMTEKWEKNRDVTMFFQKISLGDCKMWLEEFL MYWEQMLDPTKPPSLAPG (SEQ ID NO: 53), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 53.

In other embodiments, the NK cell engager is a ligand of DNAM1 chosen from NECTIN2 or NECL5, e.g., wherein:
(i) NECTIN2 comprises the amino acid sequence: QDVRVQVLPEVRGQLGGTVELPCHLLPPVPGLYISLVTWQRPDAPANHQNVAAFHPKM GPSFPSPKPGSERLSFVSAKQSTGQDTEAELQDATLALHGLTVEDEGNYTCEFATFPKGS VRGMTWLRVIAKPKNQAEAQKVTFSQDPTTVALCISKEGRPPARISWLSSLDWEAKETQ VSGTLAGTVTVTSRFTLVPSGRADGVTVTCKVEHESFEEPALIPVTLSVRYPPEVSISGYD DNWYLGRTDATLSCDVRSNPEPTGYDWSTTSGTFPTSAVAQGSQLVIHAVDSLFNTTFV CTVTNAVGMGRAEQVIFVRETPNTAGAGATGG (SEQ ID NO: 54), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 54; or
(ii) NECL5 comprises the amino acid sequence: WPPPGTGDVVVQAPTQVPGFLGDSVTLPCYLQVPNMEVTHVSQLTWARHGESGSMAV FHQTQGPSYSESKRLEFVAARLGAELRNASLRMFGLRVEDEGNYTCLFVTFPQGSRSVD IWLRVLAKPQNTAEVQKVQLTGEPVPMARCVSTGGRPPAQITWHSDLGGMPNTSQVPG FLSGTVTVTSLWILVPSSQVDGKNVTCKVEHESFEKPQLLTVNLTVYYPPEVSISGYDNN WYLGQNEATLTCDARSNPEPTGYNWSTTMGPLPPFAVAQGAQLLIRPVDKPINTTLICN VTNALGARQAELTVQVKEGPPSEHSGISRN (SEQ ID NO: 55), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 55.

In yet other embodiments, the NK cell engager is a ligand of DAP10, which is an adapter for NKG2D (see e.g., Proc Natl Acad Sci USA. 2005 May 24; 102(21): 7641-7646; and Blood, 15 Sep. 2011 Volume 118, Number 11, the full contents of each of which is incorporated by reference herein).

In other embodiments, the NK cell engager is a ligand of CD16, which is a CD16a/b ligand, e.g., a CD16a/b ligand further comprising an antibody Fc region (see e.g., Front Immunol. 2013; 4: 76 discusses how antibodies use the Fc to trigger NK cells through CD16, the full contents of which are incorporated herein).

In other embodiments, the NK cell engager is a ligand of CRTAM, which is NECL2, e.g., wherein NECL2 comprises the amino acid sequence: QNLFTKDVTVIEGEVATISCQVNKSDDSVIQLLNPNRQTIYFRDFRPLKDSRFQLLNFSSS ELKVSLTNVSISDEGRYFCQLYTDPPQESYTTITVLVPPRNLMIDIQKDTAVEGEEIEVNC TAMASKPATTIRWFKGNTELKGKSEVEEWSDMYTVTSQLMLKVHKEDDGVPVICQVE HPAVTGNLQTQRYLEVQYKPQVHIQMTYPLQGLTREGDALELTCEAIGKPQPVMVTWV RVDDEMPQHAVLSGPNLFINNLNKTDNGTYRCEASNIVGKAHSDYMLYVYDPPTTIPPP TTTTTTTTTTTTTILTIITDSRAGEEGSIRAVDH (SEQ ID NO: 56), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 56.

In other embodiments, the NK cell engager is a ligand of CD27, which is CD70, e.g., wherein CD70 comprises the amino acid sequence: QRFAQAQQQLPLESLGWDVAELQLNHTGPQQDPRLYWQGGPALGRSFLHGPELDKGQ LRIHRDGIYMVHIQVTLAICSSTTASRHHPTTLAVGICSPASRSISLLRLSFHQGCTIASQR LTPLARGDTLCTNLTGTLLPSRNTDETFFGVQWVRP (SEQ ID NO: 57), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 57.

In other embodiments, the NK cell engager is a ligand of PSGL1, which is L-selectin (CD62L), e.g., wherein L-selectin comprises the amino acid sequence: WTYHYSEKPMNWQRARRFCRDNYTDLVAIQNKAEIEYLEKTLPFSRSYYWIGIRKIGGI WTWVGTNKSLTEEAENWGDGEPNNKKNKEDCVEIYIKRNKDAGKWNDDACHKLKAA LCYTASCQPWSCSGHGECVEIINNYTCNCDVGYYGPQCQFVIQCEPLEAPELGTMDCTH PLGNFSFSSQCAFSCSEGTNLTGIEETTCGPFGNWSSPEPTCQVIQCEPLSAPDLGIMNCSH PLASFSFTSACTFICSEGTELIGKKKTICESSGIWSNPSPICQKLDKSFSMIKEGDYN (SEQ ID NO: 58), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 58.

In other embodiments, the NK cell engager is a ligand of CD96, which is NECL5, e.g., wherein NECL5 comprises the amino acid sequence: WPPPGTGDVVVQAPTQVPGFLGDSVTLPCYLQVPNMEVTHVSQLTWARHGESGSMAV FHQTQGPSYSESKRLEFVAARLGAELRNASLRMFGLRVEDEGNYTCLFVTFPQGSRSVD IWLRVLAKPQNTAEVQKVQLTGEPVPMARCVSTGGRPPAQITWHSDLGGMPNTSQVPG FLSGTVTVTSLWILVPSSQVDGKNVTCKVEHESFEKPQLLTVNLTVYYPPEVSISGYDNN WYLGQNEATLTCDARSNPEPTGYNWSTTMGPLPPFAVAQGAQLLIRPVDKPINTTLICN VTNALGARQAELTVQVKEGPPSEHSGISRN (SEQ ID NO: 55), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 55.

In other embodiments, the NK cell engager is a ligand of CD100 (SEMA4D), which is CD72, e.g., wherein CD72 comprises the amino acid sequence: RYLQVSQQLQQTNRVLEVTNSSLRQQLRLKITQLGQSAEDLQGSRRELAQSQEALQVEQ RAHQAAEGQLQACQADRQKTKETLQSEEQQRRALEQKLSNMENRLKPFFTCGSADTCC PSGWIMHQKSCFYISLTSKNWQESQKQCETLSSKLATFSEIYPQSHSYYFLNSLLPNGGS GNSYWTGLSSNKDWKLTDDTQRTRTYAQSSKCNKVHKTWSWWTLESESCRSSLPYICE MTAFRFPD (SEQ ID NO: 59), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 59.

In other embodiments, the NK cell engager is a ligand of NKp80, which is CLEC2B (AICL), e.g., wherein CLEC2B (AICL) comprises the amino acid sequence: KLTRDSQSLCPYDWIGFQNKCYYFSKEEGDWNSSKYNCSTQHADLTIIDNIEEMNFLRR YKCSSDHWIGLKMAKNRTGQWVDGATFTKSFGMRGSEGCAYLSDDGAATARCYTER KWICRKRIH (SEQ ID NO: 60), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 60.

In other embodiments, the NK cell engager is a ligand of CD244, which is CD48, e.g., wherein CD48 comprises the amino acid sequence: QGHLVHMTVVSGSNVTLNISESLPENYKQLTWFYTFDQKIVEWDSRKSKYFESKFKGR VRLDPQSGALYISKVQKEDNSTYIMRVLKKTGNEQEWKIKLQVLDPVPKPVIKIEKIEDM DDNCYLKLSCVIPGESVNYTWYGDKRPFPKELQNSVLETTLMPHNYSRCYTCQVSNSVS SKNGTVCLSPPCTLARS (SEQ ID NO: 61), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 61.

### T Cell Engagers

The present disclosure provides, inter alia, multispecific (e.g., bi-, tri-, quad-specific) or multifunctional molecules, that are engineered to contain one or more T cell engager that mediate binding to and/or activation of a T cell. Accordingly, in some embodiments, the T cell engager is selected from an antigen binding domain or ligand that binds to (e.g., and in some embodiments activates) one or more of CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, or CD226. In other embodiments, the T cell engager is selected from an antigen binding domain or ligand that binds to and does not activate one or more of CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, or CD226. In some embodiments, the T cell engager binds to CD3.

### B Cell, Macrophage & Dendritic Cell Engagers

Broadly, B cells, also known as B lymphocytes, are a type of white blood cell of the lymphocyte subtype. They function in the humoral immunity component of the adaptive immune system by secreting antibodies. Additionally, B cells present antigen (they are also classified as professional antigen-presenting cells (APCs)) and secrete cytokines. Macrophages are a type of white blood cell that engulfs and digests cellular debris, foreign substances, microbes, cancer cells via phagocytosis. Besides phagocytosis, they play important roles in nonspecific defense (innate immunity) and also help initiate specific defense mechanisms (adaptive immunity) by recruiting other immune cells such as lymphocytes. For example, they are important as antigen presenters to T cells. Beyond increasing inflammation and stimulating the immune system, macrophages also play an important anti-inflammatory role and can decrease immune reactions through the release of cytokines. Dendritic cells (DCs) are antigen-presenting cells that function in processing antigen material and present it on the cell surface to the T cells of the immune system.

The present disclosure provides, inter alia, multispecific (e.g., bi-, tri-, quad-specific) or multifunctional molecules, that include, e.g., are engineered to contain, one or more B cell, macrophage, and/or dendritic cell engager that mediate binding to and/or activation of a B cell, macrophage, and/or dendritic cell.

Accordingly, in some embodiments, the immune cell engager comprises a B cell, macrophage, and/or dendritic cell engager chosen from one or more of CD40 ligand (CD40L) or a CD70 ligand; an antibody molecule that binds to CD40 or CD70; an antibody molecule to OX40; an OX40 ligand (OX40L); an agonist of a Toll-like receptor (e.g., as described herein, e.g., a TLR4, e.g., a constitutively active TLR4 (caTLR4), or a TLR9 agonists); a 41BB; a CD2; a CD47; or a STING agonist, or a combination thereof.

In some embodiments, the B cell engager is a CD40L, an OX40L, or a CD70 ligand, or an antibody molecule that binds to OX40, CD40 or CD70.

In some embodiments, the macrophage engager is a CD2 agonist. In some embodiments, the macrophage engager is an antigen binding domain that binds to: CD40L or antigen binding domain or ligand that binds CD40, a Toll like receptor (TLR) agonist (e.g., as described herein), e.g., a TLR9 or TLR4 (e.g., caTLR4 (constitutively active TLR4), CD47, or a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In some embodiments, the dendritic cell engager is a CD2 agonist. In some embodiments, the dendritic cell engager is a ligand, a receptor agonist, or an antibody molecule that binds to one or more of: OX40L, 41BB, a TLR agonist (e.g., as described herein) (e.g., TLR9 agonist, TLR4 (e.g., caTLR4 (constitutively active TLR4)), CD47, or and a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In other embodiments, the immune cell engager mediates binding to, or activation of, one or more of a B cell, a macrophage, and/or a dendritic cell. Exemplary B cell, macrophage, and/or dendritic cell engagers can be chosen from one or more of CD40 ligand (CD40L) or a CD70 ligand; an antibody molecule that binds to CD40 or CD70; an antibody molecule to OX40; an OX40 ligand (OX40L); a Toll-like receptor agonist (e.g., a TLR4, e.g., a constitutively active TLR4 (caTLR4) or a TLR9 agonist); a 41BB agonist; a CD2; a CD47; or a STING agonist, or a combination thereof.

In some embodiments, the B cell engager is chosen from one or more of a CD40L, an OX40L, or a CD70 ligand, or an antibody molecule that binds to OX40, CD40 or CD70.

In other embodiments, the macrophage cell engager is chosen from one or more of a CD2 agonist; a CD40L; an OX40L; an antibody molecule that binds to OX40, CD40 or CD70; a Toll-like receptor agonist or a fragment thereof (e.g., a TLR4, e.g., a constitutively active TLR4 (caTLR4)); a CD47 agonist; or a STING agonist.

In other embodiments, the dendritic cell engager is chosen from one or more of a CD2 agonist, an OX40 antibody, an OX40L, 41BB agonist, a Toll-like receptor agonist or a fragment thereof (e.g., a TLR4, e.g., a constitutively active TLR4 (caTLR4)), CD47 agonist, or a STING agonist.

In one embodiment, the OX40L comprises the amino acid sequence: QVSHRYPRIQSIKVQFTEYKKEKGFILTSQKEDEIMKVQNNSVIINCDGFYLISLKGYFSQ EVNISLHYQKDEEPLFQLKKVRSVNSLMVASLTYKDKVYLNVTTDNTSLDDFHVNGGE LILIHQNPGEFCVL (SEQ ID NO: 62), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 62.

In another embodiment, the CD40L comprises the amino acid sequence: MQKGDQNPQIAAHVISEASSKTTSVLQWAEKGYYTMSNNLVTLENGKQLTVKRQGLY YIYAQVTFCSNREASSQAPFIASLCLKSPGRFERILLRAANTHSSAKPCGQQSIIHLGGVFE LQPGASVFVNVTDPSQVSHGTGFTSFGLLKL (SEQ ID NO: 63), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 63.

In yet other embodiments, the STING agonist comprises a cyclic dinucleotide, e.g., a cyclic di-GMP (cdGMP), a cyclic di-AMP (cdAMP), or a combination thereof, optionally with 2′,5′ or 3′,5′ phosphate linkages.

In one embodiment, the immune cell engager includes 41BB ligand, e.g., comprising the amino acid sequence: ACPWAVSGARASPGSAASPRLREGPELSPDDPAGLLDLRQGMFAQLVAQNVLLIDGPLS WYSDPGLAGVSLTGGLSYKEDTKELVVAKAGVYYVFFQLELRRVVAGEGSGSVSLALH LQPLRSAAGAAALALTVDLPPASSEARNSAFGFQGRLLHLSAGQRLGVHLHTEARARH AWQLTQGATVLGLFRVTPEIPAGLPSPRSE (SEQ ID NO: 64), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 64.

### Tol-Like Receptors

Toll-Like Receptors (TLRs) are evolutionarily conserved receptors are homologues of the Drosophila Toll protein, and recognize highly conserved structural motifs known as pathogen-associated microbial patterns (PAMPs), which are exclusively expressed by microbial pathogens, or danger-associated molecular patterns (DAMPs) that are endogenous molecules released from necrotic or dying cells. PAMPs include various bacterial cell wall components such as lipopolysaccharide (LPS), peptidoglycan (PGN) and lipopeptides, as well as flagellin, bacterial DNA and viral double-stranded RNA. DAMPs include intracellular proteins such as heat shock proteins as well as protein fragments from the extracellular matrix. Stimulation of TLRs by the corresponding PAMPs or DAMPs initiates signaling cascades leading to the activation of transcription factors, such as AP-1, NF-κB and interferon regulatory factors (IRFs). Signaling by TLRs results in a variety of cellular responses, including the production of interferons (IFNs), pro-inflammatory cytokines and effector cytokines that direct the adaptive immune response. TLRs are implicated in a number of inflammatory and immune disorders and play a role in cancer (Rakoff-Nahoum S. & Medzhitov R., 2009. Toll-like receptors and cancer. Nat Revs Cancer 9:57-63.)

TLRs are type I transmembrane proteins characterized by an extracellular domain containing leucine-rich repeats (LRRs) and a cytoplasmic tail that contains a conserved region called the Toll/IL-1 receptor (TTR) domain. Ten human and twelve murine TLRs have been characterized, TLR1 to TLR10 in humans, and TLR1 to TLR9, TLR11, TLR12 and TLR13 in mice, the homolog of TLR10 being a pseudogene. TLR2 is essential for the recognition of a variety of PAMPs from Gram-positive bacteria, including bacterial lipoproteins, lipomannans and lipoteichoic acids. TLR3 is implicated in virus-derived double-stranded RNA. TLR4 is predominantly activated by lipopolysaccharide. TLR5 detects bacterial flagellin and TLR9 is required for response to unmethylated CpG DNA. Finally, TLR7 and TLR8 recognize small synthetic antiviral molecules, and single-stranded RNA was reported to be their natural ligand. TLR11 has been reported to recognize uropathogenic E. coli and a profilin-like protein from Toxoplasma gondii. The repertoire of specificities of the TLRs is apparently extended by the ability of TLRs to heterodimerize with one another. For example, dimers of TLR2 and TLR6 are required for responses to diacylated lipoproteins while TLR2 and TLR1 interact to recognize triacylated lipoproteins. Specificities of the TLRs are also influenced by various adapter and accessory molecules, such as MD-2 and CD14 that form a complex with TLR4 in response to LPS.

TLR signaling consists of at least two distinct pathways: a MyD88-dependent pathway that leads to the production of inflammatory cytokines, and a MyD88-independent pathway associated with the stimulation of IFN-β and the maturation of dendritic cells. The MyD88-dependent pathway is common to all TLRs, except TLR3 (Adachi O. et al., 1998. Targeted disruption of the MyD88 gene results in loss of IL-1- and IL-18-mediated function. Immunity. 9(1):143-50). Upon activation by PAMPs or DAMPs, TLRs hetero- or homodimerize inducing the recruitment of adaptor proteins via the cytoplasmic TIR domain. Individual TLRs induce different signaling responses by usage of the different adaptor molecules. TLR4 and TLR2 signaling requires the adaptor TIRAP/Mal, which is involved in the MyD88-dependent pathway. TLR3 triggers the production of IFN-β in response to double-stranded RNA, in a MyD88-independent manner, through the adaptor TRIF/TICAM-1. TRAM/TICAM-2 is another adaptor molecule involved in the MyD88-independent pathway which function is restricted to the TLR4 pathway.

TLR3, TLR7, TLR8 and TLR9 recognize viral nucleic acids and induce type I IFNs. The signaling mechanisms leading to the induction of type I IFNs differ depending on the TLR activated. They involve the interferon regulatory factors, IRFs, a family of transcription factors known to play a critical role in antiviral defense, cell growth and immune regulation. Three IRFs (IRF3, IRF5 and IRF7) function as direct transducers of virus-mediated TLR signaling. TLR3 and TLR4 activate IRF3 and IRF7, while TLR7 and TLR8 activate IRF5 and IRF7 (Doyle S. et al., 2002. IRF3 mediates a TLR3/TLR4-specific antiviral gene program. Immunity. 17(3):251-63). Furthermore, type I IFN production stimulated by TLR9 ligand CpG-A has been shown to be mediated by PI(3)K and mTOR (Costa-Mattioli M. & Sonenberg N. 2008. RAPping production of type I interferon in pDCs through mTOR. Nature Immunol. 9: 1097-1099).

### TLR-9

TLR9 recognizes unmethylated CpG sequences in DNA molecules. CpG sites are relatively rare (˜1%) on vertebrate genomes in comparison to bacterial genomes or viral DNA. TLR9 is expressed by numerous cells of the immune system such as B lymphocytes, monocytes, natural killer (NK) cells, and plasmacytoid dendritic cells. TLR9 is expressed intracellularly, within the endosomal compartments and functions to alert the immune system of viral and bacterial infections by binding to DNA rich in CpG motifs. TLR9 signals leads to activation of the cells initiating pro-inflammatory reactions that result in the production of cytokines such as type-I interferon and IL-12.

### TLR Agonists

A TLR agonist can agonize one or more TLR, e.g., one or more of human TLR-1, 2, 3, 4, 5, 6, 7, 8, 9, or 10. In some embodiments, an adjunctive agent described herein is a TLR agonist. In some embodiments, the TLR agonist specifically agonizes human TLR-9. In some embodiments, the TLR-9 agonist is a CpG moiety. As used herein, a CpG moiety, is a linear dinucleotide having the sequence: 5′-C-phosphate-G-3′, that is, cytosine and guanine separated by only one phosphate.

In some embodiments, the CpG moiety comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more CpG dinucleotides. In some embodiments, the CpG moiety consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 CpG dinucleotides. In some embodiments, the CpG moiety has 1-5, 1-10, 1-20, 1-30, 1-40, 1-50, 5-10, 5-20, 5-30, 10-20, 10-30, 10-40, or 10-50 CpG dinucleotides.

In some embodiments, the TLR-9 agonist is a synthetic ODN (oligodeoxynucleotides). CpG ODNs are short synthetic single-stranded DNA molecules containing unmethylated CpG dinucleotides in particular sequence contexts (CpG motifs). CpG ODNs possess a partially or completely phosphorothioated (PS) backbone, as opposed to the natural phosphodiester (PO) backbone found in genomic bacterial DNA. There are three major classes of CpG ODNs: classes A, B and C, which differ in their immunostimulatory activities. CpG-A ODNs are characterized by a PO central CpG-containing palindromic motif and a PS-modified 3′ poly-G string. They induce high IFN-α production from pDCs but are weak stimulators of TLR9-dependent NF-κB signaling and pro-inflammatory cytokine (e.g. IL-6) production. CpG-B ODNs contain a full PS backbone with one or more CpG dinucleotides. They strongly activate B cells and TLR9-dependent NF-κB signaling but weakly stimulate IFN-α secretion. CpG-C ODNs combine features of both classes A and B. They contain a complete PS backbone and a CpG-containing palindromic motif C-Class CpG ODNs induce strong IFN-α production from pDC as well as B cell stimulation.

### Stromal Modifying Moieties

Solid tumors have a distinct structure that mimics that of normal tissues and comprises two distinct but interdependent compartments: the parenchyma (neoplastic cells) and the stroma that the neoplastic cells induce and in which they are dispersed. All tumors have stroma and require stroma for nutritional support and for the removal of waste products. In the case of tumors which grow as cell suspensions (e.g., leukemias, ascites tumors), the blood plasma serves as stroma (Connolly J L et al. Tumor Structure and Tumor Stroma Generation. In: Kufe D W et al., editors. Holland-Frei Cancer Medicine. 6th edition. Hamilton: BC Decker; 2003). The stroma includes a variety of cell types, including fibroblasts/myofibroblasts, glial, epithelial, fat, vascular, smooth muscle, and immune cells along with extracellular matrix (ECM) and extracellular molecules (Li Hanchen et al. Tumor Microenvironment: The Role of the Tumor Stroma in Cancer. J of Cellular Biochemistry 101: 805-815 (2007)).

Stromal modifying moieties described herein include moieties (e.g., proteins, e.g., enzymes) capable of degrading a component of the stroma, e.g., an ECM component, e.g., a glycosaminoglycan, e.g., hyaluronan (also known as hyaluronic acid or HA), chondroitin sulfate, chondroitin, dermatan sulfate, heparin sulfate, heparin, entactin, tenascin, aggrecan and keratin sulfate; or an extracellular protein, e.g., collagen, laminin, elastin, fibrinogen, fibronectin, and vitronectin.

### Stromal Modifying Enzymes

In some embodiments, the stromal modifying moiety is an enzyme. For example, the stromal modifying moiety can include, but is not limited to a hyaluronidase, a collagenase, a chondroitinase, a matrix metalloproteinase (e.g., macrophage metalloelastase).

### Hyaluronidases

Hyaluronidases are a group of neutral- and acid-active enzymes found throughout the animal kingdom. Hyaluronidases vary with respect to substrate specificity, and mechanism of action. There are three general classes of hyaluronidases: (1) Mammalian-type hyaluronidases, (EC 3.2.1.35) which are endo-beta-N-acetylhexosaminidases with tetrasaccharides and hexasaccharides as the major end products. They have both hydrolytic and transglycosidase activities, and can degrade hyaluronan and chondroitin sulfates; (2) Bacterial hyaluronidases (EC 4.2.99.1) degrade hyaluronan and, and to various extents, chondroitin sulfate and dermatan sulfate. They are endo-beta-N-acetylhexosaminidases that operate by a beta elimination reaction that yields primarily disaccharide end products; (3) Hyaluronidases (EC 3.2.1.36) from leeches, other parasites, and crustaceans are endo-beta-glucuronidases that generate tetrasaccharide and hexasaccharide end products through hydrolysis of the beta 1-3 linkage.

Mammalian hyaluronidases can be further divided into two groups: (1) neutral active and (2) acid active enzymes. There are six hyaluronidase-like genes in the human genome, HYAL1, HYAL2, HYAL3 HYAL4 HYALPI and PH20/SPAM1. HYALPI is a pseudogene, and HYAL3 has not been shown to possess enzyme activity toward any known substrates. HYAL4 is a chondroitinase and lacks activity towards hyaluronan. HYAL1 is the prototypical acid-active enzyme and PH20 is the prototypical neutral-active enzyme. Acid active hyaluronidases, such as HYAL1 and HYAL2 lack catalytic activity at neutral pH. For example, HYAL1 has no catalytic activity in vitro over pH 4.5 (Frost and Stern, “A Microtiter-Based Assay for Hyaluronidase Activity Not Requiring Specialized Reagents”, Analytical Biochemistry, vol. 251, pp. 263-269 (1997). HYAL2 is an acid active enzyme with a very low specific activity in vitro.

In some embodiments the hyaluronidase is a mammalian hyaluronidase. In some embodiments the hyaluronidase is a recombinant human hyaluronidase. In some embodiments, the hyaluronidase is a neutral active hyaluronidase. In some embodiments, the hyaluronidase is a neutral active soluble hyaluronidase. In some embodiments, the hyaluronidase is a recombinant PH20 neutral-active enzyme. In some embodiments, the hyaluronidase is a recombinant PH20 neutral-active soluble enzyme. In some embodiments the hyaluronidase is glycosylated. In some embodiments, the hyaluronidase possesses at least one N-linked glycan. A recombinant hyaluronidase can be produced using conventional methods known to those of skill in the art, e.g., U.S. Pat. No. 7,767,429, the entire contents of which are incorporated by reference herein.

In some embodiments the hyaluronidase is rHuPH20 (also referred to as Hylenex®; presently manufactured by Halozyme; approved by the FDA in 2005 (see e.g., Scodeller P (2014) Hyaluronidase and other Extracellular Matrix Degrading Enzymes for Cancer Therapy: New Uses and Nano-Formulations. J Carcinog Mutage 5:178; U.S. Pat. Nos. 7,767,429; 8,202,517; 7,431,380; 8,450,470; 8,772,246; 8,580,252, the entire contents of each of which is incorporated by reference herein). rHuPH20 is produced by genetically engineered CHO cells containing a DNA plasmid encoding for a soluble fragment of human hyaluronidase PH20. In some embodiments the hyaluronidase is glycosylated. In some embodiments, the hyaluronidase possesses at least one N-linked glycan. A recombinant hyaluronidase can be produced using conventional methods known to those of skill in the art, e.g., U.S. Pat. No. 7,767,429, the entire contents of which are incorporated by reference herein. In some embodiments, rHuPH20 has a sequence at least 95% (e.g., at least 96%, 97%, 98%, 99%, 100%) identical to the amino acid sequence of

(SEQ ID NO: 65)
LNFRAPPV1PNVPFLWAWNAPSEFCLGKFDEPLDMSLFSFIGSPRINATG
QGVTIFYVDRLGYYPYIDSITGVTVNGGIPQKISLQDHLDKAKKDITFYM
PVDNLGMAVIDWEEWRPTWARNWKPKDVYKNRSIELVQQQNVQLSLTEAT
EKAKQEFEKAGKDFLVETIKLGKLLRPNHLWGYYLFPDCYNHHYKKPGYN
GSCFNVEIKRNDDLSWLWNESTALYPSIYLNTQQSPVAATLYVRNRVREA
IRVSKIPDAKSPLPVFAYTRIVFTDQVLKFLSQDELVYTFGETVALGASG
IVIWGTLSIMRSMKSCLLLDNYMETILNPYIINVTLAAKMCSQVLCQEQG
VCIRKNWNSSDYLHLNPDNFAIQLEKGGKFTVRGKPTLEDLEQFSEKFYC
SCYSTLSCKEKADVKDTDAVDVCIADGVCIDAFLKPPMETEEPQIFYNAS
PSTLS.

In any of the methods provided herein, the anti-hyaluronan agent can be an agent that degrades hyaluronan or can be an agent that inhibits the synthesis of hyaluronan. For example, the anti-hyaluronan agent can be a hyaluronan degrading enzyme. In another example, the anti-hyaluronan agent or is an agent that inhibits hyaluronan synthesis. For example, the anti-hyaluronan agent is an agent that inhibits hyaluronan synthesis such as a sense or antisense nucleic acid molecule against an HA synthase or is a small molecule drug. For example, an anti-hyaluronan agent is 4-methylumbelliferone (MU) or a derivative thereof, or leflunomide or a derivative thereof. Such derivatives include, for example, a derivative of 4-methylumbelliferone (MU) that is 6,7-dihydroxy-4-methyl coumarin or 5,7-dihydroxy-4-methyl coumarin.

In further examples of the methods provided herein, the hyaluronan degrading enzyme is a hyaluronidase. In some examples, the hyaluronan-degrading enzyme is a PH20 hyaluronidase or truncated form thereof to lacking a C-terminal glycosylphosphatidylinositol (GPI) attachment site or a portion of the GPI attachment site. In specific examples, the hyaluronidase is a PH20 selected from a human, monkey, bovine, ovine, rat, mouse or guinea pig PH20. For example, the hyaluronan-degrading enzyme is a human PH20 hyaluronidase that is neutral active and N-glycosylated and is selected from among (a) a hyaluronidase polypeptide that is a full-length PH20 or is a C-terminal truncated form of the PH20, wherein the truncated form includes at least amino acid residues 36-464 of SEQ ID NO: 65, such as 36-481, 36-482, 36-483, where the full-length PH20 has the sequence of amino acids set forth in SEQ ID NO: 65; or (b) a hyaluronidase polypeptide comprising a sequence of amino acids having at least 85%, 86%, 87%, 88%, 89%, 90%, 91%, 92%, 93%, 94%, 95%, 96%, 97%, 98%, 99% or more sequence identity with the polypeptide or truncated form of sequence of amino acids set forth in SEQ ID NO: 65; or (c) a hyaluronidase polypeptide of (a) or (b) comprising amino acid substitutions, whereby the hyaluronidase polypeptide has a sequence of amino acids having at least 85%, 86%, 87%, 88%, 89%, 90%, 91%, 92%, 93%, 94%, 95%, 96%, 97%, 98%, 99% or more sequence identity with the polypeptide set forth in SEQ ID NO: 65 or the with the corresponding truncated forms thereof. In exemplary examples, the hyaluronan-degrading enzyme is a PH20 that comprises a composition designated rHuPH20.

In other examples, the anti-hyaluronan agent is a hyaluronan degrading enzyme that is modified by conjugation to a polymer. The polymer can be a PEG and the anti-hyaluronan agent a PEGylated hyaluronan degrading enzyme. Hence, in some examples of the methods provided herein the hyaluronan-degrading enzyme is modified by conjugation to a polymer. For example, the hyaluronan-degrading enzyme is conjugated to a PEG, thus the hyaluronan degrading enzyme is PEGylated. In an exemplary example, the hyaluronan-degrading enzyme is a PEGylated PH20 enzyme (PEGPH20). In the methods provided herein, the corticosteroid can be a glucocorticoid that is selected from among cortisones, dexamethasones, hydrocortisones, methylprednisolones, prednisolones and prednisones.

### Chondroitinases

Chondroitinases are enzymes found throughout the animal kingdom which degrade glycosaminoglycans, specifically chondroitins and chondroitin sulfates, through an endoglycosidase reaction. In some embodiments the chondroitinase is a mammalian chondroitinase. In some embodiments the chondroitinase is a recombinant human chondroitinase. In some embodiments the chondroitinase is HYAL4. Other exemplary chondroitinases include chondroitinase ABC (derived from Proteus vulgaris; Japanese Patent Application Laid-open No 6-153947, T. Yamagata et al. J. Biol. Chem., 243, 1523 (1968), S. Suzuki et al, J. Biol. Chem., 243, 1543 (1968)), chondroitinase AC (derived from Flavobacterium heparinum; T. Yamagata et al., J. Biol. Chem., 243, 1523 (1968)), chondroitinase AC II (derived from Arthrobacter aurescens; K. Hiyama, and S. Okada, J. Biol. Chem., 250, 1824 (1975), K. Hiyama and S. Okada, J. Biochem. (Tokyo), 80, 1201 (1976)), Hyaluronidase ACIII (derived from Flavobacterium sp. Hp102; Hirofumi Miyazono et al., Seikagaku, 61, 1023 (1989)), chondroitinase B (derived from Flavobacterium heparinum; Y. M. Michelacci and C. P. Dietrich, Biochem. Biophys. Res. Commun., 56, 973 (1974), Y. M. Michelacci and C. P. Dietrich, Biochem. J., 151, 121 (1975), Kenichi Maeyama et al, Seikagaku, 57, 1189 (1985)), chondroitinase C (derived from Flavobacterium sp. Hp102; Hirofumi Miyazono et al, Seikagaku, 61, 1023 (1939)), and the like.

### Matrix Metalloproteinases

Matrix metalloproteases (MMPs) are zinc-dependent endopeptidases that are the major proteases involved in extracellular matrix (ECM) degradation. MMPs are capable of degrading a wide range of extracellular molecules and a number of bioactive molecules. Twenty-four MMP genes have been identified in humans, which can be organized into six groups based on domain organization and substrate preference: Collagenases (MMP-1, -8 and -13), Gelatinases (MMP-2 and MMP-9), Stromelysins (MMP-3, -10 and -11), Matrilysin (MMP-7 and MMP-26), Membrane-type (MT)-MMPs (MMP-14, -15, -16, -17, -24 and -25) and others (MMP-12, -19, -20, -21, -23, -27 and -28). In some embodiments, the stromal modifying moiety is a human recombinant MMP (e.g., MMP-1, -2, -3, -4, -5, -6, -7, -8, -9, 10, -11, -12, -13, -14, 15, -15, -17, -18, -19, 20, -21, -22, -23, or -24).

### Collagenases

The three mammalian collagenases (MMP-1, -8, and -13) are the principal secreted endopeptidases capable of cleaving collagenous extracellular matrix. In addition to fibrillar collagens, collagenases can cleave several other matrix and non-matrix proteins including growth factors. Collagenases are synthesized as inactive pro-forms, and once activated, their activity is inhibited by specific tissue inhibitors of metalloproteinases, TIMPs, as well as by non-specific proteinase inhibitors (Ala-aho R et al. Biochimie. Collagenases in cancer. 2005 March-April; 87(3-4):273-86). In some embodiments, the stromal modifying moiety is a collagenase. In some embodiments, the collagenase is a human recombinant collagenase. In some embodiments, the collagenase is MMP-1. In some embodiments, the collagenase is MMP-8. In some embodiments, the collagenase is MMP-13.

### Macrophage Metalloelastase

Macrophage metalloelastase (MME), also known as MMP-12, is a member of the stromelysin subgroup of MMPs and catalyzes the hydrolysis of soluble and insoluble elastin and a broad selection of matrix and nonmatrix substrates including type IV collagen, fibronectin, laminin, vitronectin, entactin, heparan, and chondroitin sulfates (Erja Kerkela et al. Journal of Investigative Dermatology (2000) 114, 1113-1119; doi:10.1046/j.1523-1747.2000.00993). In some embodiments, the stromal modifying moiety is a MME. In some embodiments, the MME is a human recombinant MME. In some embodiments, the MME is MMP-12.

### Additional Stromal Modifying Moieties

In some embodiments, the stromal modifying moiety causes one or more of: decreases the level or production of a stromal or extracellular matrix (ECM) component; decreases tumor fibrosis; increases interstitial tumor transport; improves tumor perfusion; expands the tumor microvasculature; decreases interstitial fluid pressure (IFP) in a tumor; or decreases or enhances penetration or diffusion of an agent, e.g., a cancer therapeutic or a cellular therapy, into a tumor or tumor vasculature.

In some embodiments, the stromal or ECM component decreased is chosen from a glycosaminoglycan or an extracellular protein, or a combination thereof. In some embodiments, the glycosaminoglycan is chosen from hyaluronan (also known as hyaluronic acid or HA), chondroitin sulfate, chondroitin, dermatan sulfate, heparin, heparin sulfate, entactin, tenascin, aggrecan and keratin sulfate. In some embodiments, the extracellular protein is chosen from collagen, laminin, elastin, fibrinogen, fibronectin, or vitronectin. In some embodiments, the stromal modifying moiety includes an enzyme molecule that degrades a tumor stroma or extracellular matrix (ECM). In some embodiments, the enzyme molecule is chosen from a hyaluronidase molecule, a collagenase molecule, a chondroitinase molecule, a matrix metalloproteinase molecule (e.g., macrophage metalloelastase), or a variant (e.g., a fragment) of any of the aforesaid. The term “enzyme molecule” includes a full length, a fragment or a variant of the enzyme, e.g., an enzyme variant that retains at least one functional property of the naturally-occurring enzyme.

In some embodiments, the stromal modifying moiety decreases the level or production of hyaluronic acid. In other embodiments, the stromal modifying moiety comprises a hyaluronan degrading enzyme, an agent that inhibits hyaluronan synthesis, or an antibody molecule against hyaluronic acid.

In some embodiments, the hyaluronan degrading enzyme is a hyaluronidase molecule, e.g., a full length or a variant (e.g., fragment thereof) thereof. In some embodiments, the hyaluronan degrading enzyme is active in neutral or acidic pH, e.g., pH of about 4-5. In some embodiments, the hyaluronidase molecule is a mammalian hyaluronidase molecule, e.g., a recombinant human hyaluronidase molecule, e.g., a full length or a variant (e.g., fragment thereof, e.g., a truncated form) thereof. In some embodiments, the hyaluronidase molecule is chosen from HYAL1, HYAL2, or PH-20/SPAM1, or a variant thereof (e.g., a truncated form thereof). In some embodiments, the truncated form lacks a C-terminal glycosylphosphatidylinositol (GPI) attachment site or a portion of the GPI attachment site. In some embodiments, the hyaluronidase molecule is glycosylated, e.g., comprises at least one N-linked glycan.

In some embodiments, the hyaluronidase molecule comprises the amino acid sequence: LNFRAPPVIPNVPFLWAWNAPSEFCLGKFDEPLDMSLFSFIGSPRINATGQGVTIFYVDR LGYYPYIDSITGVTVNGGIPQKISLQDHLDKAKKDITFYMPVDNLGMAVIDWEEWRPTW ARNWKPKDVYKNRSIELVQQQNVQLSLTEATEKAKQEFEKAGKDFLVETIKLGKLLRP NHLWGYYLFPDCYNHHYKKPGYNGSCFNVEIKRNDDLSWLWNESTALYPSIYLNTQQS PVAATLYVRNRVREAIRVSKIPDAKSPLPVFAYTRIVFTDQVLKFLSQDELVYTFGETVA LGASGIVIWGTLSIMRSMKSCLLLDNYMETILNPYIINVTLAAKMCSQVLCQEQGVCIRK NWNSSDYLHLNPDNFAIQLEKGGKFTVRGKPTLEDLEQFSEKFYCSCYSTLSCKEKADV KDTDAVDVCIADGVCIDAFLKPPMETEEPQIFYNASPSTLS (SEQ ID NO:66), or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 66.

In some embodiments, the hyaluronidase molecule comprises:
(i) the amino acid sequence of 36-464 of SEQ ID NO: 66;
(ii) the amino acid sequence of 36-481, 36-482, or 36-483 of PH20, wherein PH20 has the sequence of amino acids set forth in SEQ ID NO: 66; or
(iii) an amino acid sequence having at least 95% to 100% sequence identity to the polypeptide or truncated form of sequence of amino acids set forth in SEQ ID NO: 66; or
(iv) an amino acid sequence having 30, 20, 10, 5 or fewer amino acid substitutions to the amino acid sequence set forth in SEQ ID NO: 66. In some embodiments, the hyaluronidase molecule comprises an amino acid sequence at least 95% (e.g., at least 95%, 96%, 97%, 98%, 99%, 100%) identical to the amino acid sequence of SEQ ID NO: 66. In some embodiments, the hyaluronidase molecule is encoded by a nucleotide sequence at least 95% (e.g., at least 96%, 97%, 98%, 99%, 100%) identical to the nucleotide sequence of SEQ ID NO: 66.

In some embodiments, the hyaluronidase molecule is PH20, e.g., rHuPH20. In some embodiments, the hyaluronidase molecule is HYAL1 and comprises the amino acid sequence: FRGPLLPNRPFTTVWNANTQWCLERHGVDVDVSVFDVVANPGQTFRGPDMTIFYSSQG TYPYYTPTGEPVFGGLPQNASLIAHLARTFQDILAAIPAPDFSGLAVIDWEAWRPRWAFN WDTKDIYRQRSRALVQAQHPDWPAPQVEAVAQDQFQGAARAWMAGTLQLGRALRPR GLWGFYGFPDCYNYDFLSPNYTGQCPSGIRAQNDQLGWLWGQSRALYPSIYMPAVLEG TGKSQMYVQHRVAEAFRVAVAAGDPNLPVLPYVQIFYDTTNHFLPLDELEHSLGESAA QGAAGVVLWVSWENTRTKESCQAIKEYMDTTLGPFILNVTSGALLCSQALCSGHGRCV RRTSHPKALLLLNPASFSIQLTPGGGPLSLRGALSLEDQAQMAVEFKCRCYPGWQAPWC ERKSMW (SEQ ID NO: 67), or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 67.

In some embodiments, the hyaluronan degrading enzyme, e.g., the hyaluronidase molecule, further comprises a polymer, e.g., is conjugated to a polymer, e.g., PEG. In some embodiments, the hyaluronan-degrading enzyme is a PEGylated PH20 enzyme (PEGPH20). In some embodiments, the hyaluronan degrading enzyme, e.g., the hyaluronidase molecule, further comprises an immunoglobulin chain constant region (e.g., Fc region) chosen from, e.g., the heavy chain constant regions of IgG1, IgG2, IgG3, and IgG4, more particularly, the heavy chain constant region of human IgG1, IgG2, IgG3, or IgG4. In some embodiments, the immunoglobulin constant region (e.g., the Fc region) is linked, e.g., covalently linked to, the hyaluronan degrading enzyme, e.g., the hyaluronidase molecule. In some embodiments, the immunoglobulin chain constant region (e.g., Fc region) is altered, e.g., mutated, to increase or decrease one or more of: Fc receptor binding, antibody glycosylation, the number of cysteine residues, effector cell function, or complement function. In some embodiments, the hyaluronan degrading enzyme, e.g., the hyaluronidase molecule forms a dimer.

In some embodiments, the stromal modifying moiety comprises an inhibitor of the synthesis of hyaluronan, e.g., an HA synthase. In some embodiments, the inhibitor comprises a sense or an antisense nucleic acid molecule against an HA synthase or is a small molecule drug.

In some embodiments, the inhibitor is 4-methylumbelliferone (MU) or a derivative thereof (e.g., 6,7-dihydroxy-4-methyl coumarin or 5,7-dihydroxy-4-methyl coumarin), or leflunomide or a derivative thereof.

In some embodiments, the stromal modifying moiety comprises antibody molecule against hyaluronic acid.

In some embodiments, the stromal modifying moiety comprises a collagenase molecule, e.g., a mammalian collagenase molecule, or a variant (e.g., fragment) thereof. In some embodiments, the collagenase molecule is collagenase molecule IV, e.g., comprising the amino acid sequence of: YNFFPRKPKWDKNQITYRIIGYTPDLDPETVDDAFARAFQVWSDVTPLRFSRIHDGEADI MINFGRWEHGDGYPFDGKDGLLAHAFAPGTGVGGDSHFDDDELWTLGEGQVVRVKY GNADGEYCKFPFLFNGKEYNSCTDTGRSDGFLWCSTTYNFEKDGKYGFCPHEALFTMG GNAEGQPCKFPFRFQGTSYDSCTTEGRTDGYRWCGTTEDYDRDKKYGFCPETAMSTVG GNSEGAPCVFPFTFLGNKYESCTSAGRSDGKMWCATTANYDDDRKWGFCPDQGYSLF LVAAHEFGHAMGLEHSQDPGALMAPIYTYTKNFRLSQDDIKGIQELYGASPDIDLGTGP TPTLGPVTPEICKQDIVFDGIAQIRGEIFFFKDRFIWRTVTPRDKPMGPLLVATFWPELPEK IDAVYEAPQEEKAVFFAGNEYWIYSASTLERGYPKPLTSLGLPPDVQRVDAAFNWSKNK KTYIFAGDKFWRYNEVKKKMDPGFPKLIADAWNAIPDNLDAVVDLQGGGHSYFFKGA YYLKLENQSLKSVKFGSIKSDWLGC (SEQ ID NO: 68), or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 68.

### Linkers

The multispecific or multifunctional molecule disclosed herein can further include a linker, e.g., a linker between one or more of: the tumor-targeting moiety and the cytokine molecule (or the modulator of a cytokine molecule), the tumor-targeting moiety and the immune cell engager, the tumor-targeting moiety and the stromal modifying moiety, the first tumor-targeting moiety and the second tumor-targeting moiety, the cytokine molecule (or the modulator of a cytokine molecule) and the immune cell engager, the cytokine molecule (or the modulator of a cytokine molecule) and the stromal modifying moiety, the immune cell engager and the stromal modifying moiety, the tumor-targeting moiety and the immunoglobulin chain constant region, the cytokine molecule (or the modulator of a cytokine molecule) and the immunoglobulin chain constant region, the immune cell engager and the immunoglobulin chain constant region, or the stromal modifying moiety and the immunoglobulin chain constant region. In embodiments, the linker is chosen from: a cleavable linker, a non-cleavable linker, a peptide linker, a flexible linker, a rigid linker, a helical linker, or a non-helical linker, or a combination thereof.

In one embodiment, the multispecific molecule can include one, two, three or four linkers, e.g., a peptide linker. In one embodiment, the peptide linker includes Gly and Ser. In some embodiments, the peptide linker comprises Gly and Ser. In some embodiments, the peptide linker is selected from GGGGS (SEQ ID NO: 69); GGGGSGGGGS (SEQ ID NO: 70); GGGGSGGGGSGGGGS (SEQ ID NO: 71); and DVPSGPGGGGGSGGGGS (SEQ ID NO: 72). In some embodiments, the peptide linker is a A(EAAAK)nA (SEQ ID NO: 201) family of linkers (e.g., as described in Protein Eng. (2001) 14 (8): 529-532). These are stiff helical linkers with n ranging from 2-5. In some embodiments, the peptide linker is selected from

(SEQ ID NO: 73)
AEAAAKEAAAKAAA;
(SEQ ID NO: 74)
AEAAAKEAAAKEAAAKAAA;
(SEQ ID NO: 75)
AEAAAKEAAAKEAAAKEAAAKAAA;
and
(SEQ ID NO: 76)
AEAAAKEAAAKEAAAKEAAAKEAAAKAAA

### Nucleic Acids

Nucleic acids encoding the aforementioned multispecific or multifunctional molecules are also disclosed.

In certain embodiments, the invention features nucleic acids comprising nucleotide sequences that encode heavy and light chain variable regions and CDRs or hypervariable loops of the antibody molecules, as described herein. For example, the invention features a first and second nucleic acid encoding heavy and light chain variable regions, respectively, of an antibody molecule chosen from one or more of the antibody molecules disclosed herein. The nucleic acid can comprise a nucleotide sequence as set forth in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein.

In certain embodiments, the nucleic acid can comprise a nucleotide sequence encoding at least one, two, or three CDRs or hypervariable loops from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In other embodiments, the nucleic acid can comprise a nucleotide sequence encoding at least one, two, or three CDRs or hypervariable loops from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In yet another embodiment, the nucleic acid can comprise a nucleotide sequence encoding at least one, two, three, four, five, or six CDRs or hypervariable loops from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs or hypervariable loops from a heavy chain variable region having the nucleotide sequence as set forth in the tables herein, a sequence substantially homologous thereto (e.g., a sequence at least about 85%, 90%, 95%, 99% or more identical thereto, and/or capable of hybridizing under the stringency conditions described herein). In another embodiment, the nucleic acid can comprise a nucleotide sequence encoding at least one, two, or three CDRs or hypervariable loops from a light chain variable region having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein). In yet another embodiment, the nucleic acid can comprise a nucleotide sequence encoding at least one, two, three, four, five, or six CDRs or hypervariable loops from heavy and light chain variable regions having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein).

In certain embodiments, the nucleic acid can comprise a nucleotide sequence encoding a cytokine molecule (or a modulator of a cytokine molecule), an immune cell engager, or a stromal modifying moiety disclosed herein.

In another aspect, the application features host cells and vectors containing the nucleic acids described herein. The nucleic acids may be present in a single vector or separate vectors present in the same host cell or separate host cell, as described in more detail hereinbelow.

### Vectors

Further provided herein are vectors comprising the nucleotide sequences encoding a multispecific or multifunctional molecule described herein. In one embodiment, the vectors comprise nucleotides encoding a multispecific or multifunctional molecule described herein. In one embodiment, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Cells

In another aspect, the application features host cells and vectors containing the nucleic acids described herein. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell. The invention also provides host cells comprising a nucleic acid encoding an antibody molecule as described herein.

In one embodiment, the host cells are genetically engineered to comprise nucleic acids encoding the antibody molecule.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The invention also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### Uses and Combination Therapies

Methods described herein include treating a cancer in a subject by using a multispecific or multifunctional molecule described herein, e.g., using a pharmaceutical composition described herein. Also provided are methods for reducing or ameliorating a symptom of a cancer in a subject, as well as methods for inhibiting the growth of a cancer and/or killing one or more cancer cells. In embodiments, the methods described herein decrease the size of a tumor and/or decrease the number of cancer cells in a subject administered with a described herein or a pharmaceutical composition described herein.

In embodiments, the cancer is a hematological cancer. In embodiments, the hematological cancer is a leukemia or a lymphoma. As used herein, a “hematologic cancer” refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sezary syndrome, Waldenstrom macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

In embodiments, the cancer is a myeloproliferative neoplasm, e.g., primary or idiopathic myelofibrosis (MF), essential thrombocytosis (ET), polycythemia vera (PV), or chronic myelogenous leukemia (CML). In embodiments, the cancer is myelofibrosis. In embodiments, the subject has myelofibrosis. In embodiments, the subject does not have the JAK2-V617F mutation. In embodiments, the subject has the JAK2-V617F mutation.

In embodiments, the cancer is a solid cancer. Exemplary solid cancers include, but are not limited to, ovarian cancer, rectal cancer, stomach cancer, testicular cancer, cancer of the anal region, uterine cancer, colon cancer, rectal cancer, renal-cell carcinoma, liver cancer, non-small cell carcinoma of the lung, cancer of the small intestine, cancer of the esophagus, melanoma, Kaposi's sarcoma, cancer of the endocrine system, cancer of the thyroid gland, cancer of the parathyroid gland, cancer of the adrenal gland, bone cancer, pancreatic cancer, skin cancer, cancer of the head or neck, cutaneous or intraocular malignant melanoma, uterine cancer, brain stem glioma, pituitary adenoma, epidermoid cancer, carcinoma of the cervix squamous cell cancer, carcinoma of the fallopian tubes, carcinoma of the endometrium, carcinoma of the vagina, sarcoma of soft tissue, cancer of the urethra, carcinoma of the vulva, cancer of the penis, cancer of the bladder, cancer of the kidney or ureter, carcinoma of the renal pelvis, spinal axis tumor, neoplasm of the central nervous system (CNS), primary CNS lymphoma, tumor angiogenesis, metastatic lesions of said cancers, or combinations thereof.

In embodiments, the multispecific or multifunctional molecules (or pharmaceutical composition) are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the pharmaceutical composition (or multispecific or multifunctional molecules) to be administered can be determined by a physician with consideration of individual differences in tumor size, extent of infection or metastasis, age, weight, and condition of the subject. In embodiments, the pharmaceutical composition described herein can be administered at a dosage of 10⁴ to 10⁹ cells/kg body weight, e.g., 10⁵ to 10⁶ cells/kg body weight, including all integer values within those ranges. In embodiments, the pharmaceutical composition described herein can be administered multiple times at these dosages. In embodiments, the pharmaceutical composition described herein can be administered using infusion techniques described in immunotherapy (see, e.g., Rosenberg et al., New Eng. J. of Med. 319:1676, 1988).

In embodiments, the multispecific or multifunctional molecules or pharmaceutical composition is administered to the subject parenterally. In embodiments, the cells are administered to the subject intravenously, subcutaneously, intratumorally, intranodally, intramuscularly, intradermally, or intraperitoneally. In embodiments, the cells are administered, e.g., injected, directly into a tumor or lymph node. In embodiments, the cells are administered as an infusion (e.g., as described in Rosenberg et al., New Eng. J. of Med. 319:1676, 1988) or an intravenous push. In embodiments, the cells are administered as an injectable depot formulation. In embodiments, the subject is a mammal. In embodiments, the subject is a human, monkey, pig, dog, cat, cow, sheep, goat, rabbit, rat, or mouse. In embodiments, the subject is a human. In embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

### Combination Therapies

The multispecific or multifunctional molecules disclosed herein can be used in combination with a second therapeutic agent or procedure.

In embodiments, the multispecific or multifunctional molecule and the second therapeutic agent or procedure are administered/performed after a subject has been diagnosed with a cancer, e.g., before the cancer has been eliminated from the subject. In embodiments, the multispecific or multifunctional molecule and the second therapeutic agent or procedure are administered/performed simultaneously or concurrently. For example, the delivery of one treatment is still occurring when the delivery of the second commences, e.g., there is an overlap in administration of the treatments. In other embodiments, the multispecific or multifunctional molecule and the second therapeutic agent or procedure are administered/performed sequentially. For example, the delivery of one treatment ceases before the delivery of the other treatment begins.

In embodiments, combination therapy can lead to more effective treatment than monotherapy with either agent alone. In embodiments, the combination of the first and second treatment is more effective (e.g., leads to a greater reduction in symptoms and/or cancer cells) than the first or second treatment alone. In embodiments, the combination therapy permits use of a lower dose of the first or the second treatment compared to the dose of the first or second treatment normally required to achieve similar effects when administered as a monotherapy. In embodiments, the combination therapy has a partially additive effect, wholly additive effect, or greater than additive effect.

In one embodiment, the multispecific or multifunctional molecule is administered in combination with a therapy, e.g., a cancer therapy (e.g., one or more of anti-cancer agents, immunotherapy, photodynamic therapy (PDT), surgery and/or radiation). The terms “chemotherapeutic,” “chemotherapeutic agent,” and “anti-cancer agent” are used interchangeably herein. The administration of the multispecific or multifunctional molecule and the therapy, e.g., the cancer therapy, can be sequential (with or without overlap) or simultaneous. Administration of the multispecific or multifunctional molecule can be continuous or intermittent during the course of therapy (e.g., cancer therapy). Certain therapies described herein can be used to treat cancers and non-cancerous diseases. For example, PDT efficacy can be enhanced in cancerous and non-cancerous conditions (e.g., tuberculosis) using the methods and compositions described herein (reviewed in, e.g., Agostinis, P. et al. (2011) CA Cancer J. Clin. 61:250-281).

### Anti-Cancer Therapies

In other embodiments, the multispecific or multifunctional molecule is administered in combination with a low or small molecular weight chemotherapeutic agent. Exemplary low or small molecular weight chemotherapeutic agents include, but not limited to, 13-cis-retinoic acid (isotretinoin, ACCUTANE®), 2-CdA (2-chlorodeoxyadenosine, cladribine, LEUSTATIN™), 5-azacitidine (azacitidine, VIDAZA®), 5-fluorouracil (5-FU, fluorouracil, ADRUCIL®), 6-mercaptopurine (6-MP, mercaptopurine, PURINETHOL®), 6-TG (6-thioguanine, thioguanine, THIOGUANINE TABLOID®), abraxane (paclitaxel protein-bound), actinomycin-D (dactinomycin, COSMEGEN®), alitretinoin (PANRETIN®), all-transretinoic acid (ATRA, tretinoin, VESANOID®), altretamine (hexamethylmelamine, HMM, HEXALEN®), amethopterin (methotrexate, methotrexate sodium, MTX, TREXALL™, RHEUMATREX®), amifostine (ETHYOL®), arabinosylcytosine (Ara-C, cytarabine, CYTOSAR-U®), arsenic trioxide (TRISENOX®), asparaginase (Erwinia L-asparaginase, L-asparaginase, ELSPAR®, KIDROLASE®), BCNU (carmustine, BiCNU®), bendamustine (TREANDA®), bexarotene (TARGRETIN®), bleomycin (BLENOXANE®), busulfan (BUSULFEX®, MYLERAN®), calcium leucovorin (Citrovorum Factor, folinic acid, leucovorin), camptothecin-11 (CPT-11, irinotecan, CAMPTOSAR®), capecitabine (XELODA®), carboplatin (PARAPLATIN®), carmustine wafer (prolifeprospan 20 with carmustine implant, GLIADEL® wafer), CCI-779 (temsirolimus, TORISEL®), CCNU (lomustine, CeeNU), CDDP (cisplatin, PLATINOL®, PLATINOL-AQ®), chlorambucil (leukeran), cyclophosphamide (CYTOXAN®, NEOSAR®), dacarbazine (DIC, DTIC, imidazole carboxamide, DTIC-DOME®), daunomycin (daunorubicin, daunorubicin hydrochloride, rubidomycin hydrochloride, CERUBIDINE®), decitabine (DACOGEN®), dexrazoxane (ZINECARD®), DHAD (mitoxantrone, NOVANTRONE®), docetaxel (TAXOTERE®), doxorubicin (ADRIAMYCIN®, RUBEX®), epirubicin (ELLENCE™), estramustine (EMCYT®), etoposide (VP-16, etoposide phosphate, TOPOSAR®, VEPESID®, ETOPOPHOS®), floxuridine (FUDR®), fludarabine (FLUDARA®), fluorouracil (cream) (CARAC™, EFUDEX®, FLUOROPLEX®), gemcitabine (GEMZAR®), hydroxyurea (HYDREA®, DROXIA™, MYLOCEL™), idarubicin (IDAMYCIN®), ifosfamide (IFEX®), ixabepilone (IXEMPRA™), LCR (leurocristine, vincristine, VCR, ONCOVIN®, VINCASAR PFS®), L-PAM (L-sarcolysin, melphalan, phenylalanine mustard, ALKERAN®), mechlorethamine (mechlorethamine hydrochloride, mustine, nitrogen mustard, MUSTARGEN®), mesna (MESNEX™), mitomycin (mitomycin-C, MTC, MUTAMYCIN®), nelarabine (ARRANON®), oxaliplatin (ELOXATIN™), paclitaxel (TAXOL®, ONXAL™), pegaspargase (PEG-L-asparaginase, ONCOSPAR®), PEMETREXED (ALIMTA®), pentostatin (NIPENT®), procarbazine (MATULANE®), streptozocin (ZANOSAR®), temozolomide (TEMODAR®), teniposide (VM-26, VUMON®), TESPA (thiophosphoamide, thiotepa, TSPA, THIOPLEX®), topotecan (HYCAMTIN®), vinblastine (vinblastine sulfate, vincaleukoblastine, VLB, ALKABAN-AQ®, VELBAN®), vinorelbine (vinorelbine tartrate, NAVELBINE®), and vorinostat (ZOLINZA®).

In another embodiment, the multispecific or multifunctional molecule is administered in conjunction with a biologic. Biologics useful in the treatment of cancers are known in the art and a binding molecule of the invention may be administered, for example, in conjunction with such known biologics. For example, the FDA has approved the following biologics for the treatment of breast cancer: HERCEPTIN® (trastuzumab, Genentech Inc., South San Francisco, Calif.; a humanized monoclonal antibody that has anti-tumor activity in HER2-positive breast cancer); FASLODEX® (fulvestrant, AstraZeneca Pharmaceuticals, LP, Wilmington, Del.; an estrogen-receptor antagonist used to treat breast cancer); ARIMIDEX® (anastrozole, AstraZeneca Pharmaceuticals, LP; a nonsteroidal aromatase inhibitor which blocks aromatase, an enzyme needed to make estrogen); Aromasin® (exemestane, Pfizer Inc., New York, N.Y.; an irreversible, steroidal aromatase inactivator used in the treatment of breast cancer); FEMARA® (letrozole, Novartis Pharmaceuticals, East Hanover, N.J.; a nonsteroidal aromatase inhibitor approved by the FDA to treat breast cancer); and NOLVADEX® (tamoxifen, AstraZeneca Pharmaceuticals, LP; a nonsteroidal antiestrogen approved by the FDA to treat breast cancer).

Other biologics with which the binding molecules of the invention may be combined include: AVASTIN® (bevacizumab, Genentech Inc.; the first FDA-approved therapy designed to inhibit angiogenesis); and ZEVALIN® (ibritumomab tiuxetan, Biogen Idec, Cambridge, Mass.; a radiolabeled monoclonal antibody currently approved for the treatment of B-cell lymphomas).

In addition, the FDA has approved the following biologics for the treatment of colorectal cancer: AVASTIN®; ERBITUX® (cetuximab, ImClone Systems Inc., New York, N.Y., and Bristol-Myers Squibb, New York, N.Y.; is a monoclonal antibody directed against the epidermal growth factor receptor (EGFR)); GLEEVEC® (imatinib mesylate; a protein kinase inhibitor); and ERGAMISOL® (levamisole hydrochloride, Janssen Pharmaceutica Products, LP, Titusville, N.J.; an immunomodulator approved by the FDA in 1990 as an adjuvant treatment in combination with 5-fluorouracil after surgical resection in patients with Dukes' Stage C colon cancer).

For the treatment of lung cancer, exemplary biologics include TARCEVA® (erlotinib HCL, OSI Pharmaceuticals Inc., Melville, N.Y.; a small molecule designed to target the human epidermal growth factor receptor 1 (HER1) pathway).

For the treatment of multiple myeloma, exemplary biologics include VELCADE® Velcade (bortezomib, Millennium Pharmaceuticals, Cambridge Mass.; a proteasome inhibitor). Additional biologics include THALIDOMID® (thalidomide, Clegene Corporation, Warren, N.J.; an immunomodulatory agent and appears to have multiple actions, including the ability to inhibit the growth and survival of myeloma cells and anti-angiogenesis).

Additional exemplary cancer therapeutic antibodies include, but are not limited to, 3F8, abagovomab, adecatumumab, afutuzumab, alacizumab pegol, alemtuzumab (CAMPATH®, MABCAMPATH®), altumomab pentetate (HYBRI-CEAKER®), anatumomab mafenatox, anrukinzumab (IMA-638), apolizumab, arcitumomab (CEA-SCAN®), bavituximab, bectumomab (LYMPHOSCAN®), belimumab (BENLYSTA®, LYMPHOSTAT-B®), besilesomab (SCINTIMUN®), bevacizumab (AVASTIN®), bivatuzumab mertansine, blinatumomab, brentuximab vedotin, cantuzumab mertansine, capromab pendetide (PROSTASCINT®), catumaxomab (REMOVAB®), CC49, cetuximab (C225, ERBITUX®), citatuzumab bogatox, cixutumumab, clivatuzumab tetraxetan, conatumumab, dacetuzumab, denosumab (PROLIA®), detumomab, ecromeximab, edrecolomab (PANOREX®), elotuzumab, epitumomab cituxetan, epratuzumab, ertumaxomab (REXOMUN®), etaracizumab, farletuzumab, figitumumab, fresolimumab, galiximab, gemtuzumab ozogamicin (MYLOTARG®), girentuximab, glembatumumab vedotin, ibritumomab (ibritumomab tiuxetan, ZEVALIN®), igovomab (INDIMACIS-125®), intetumumab, inotuzumab ozogamicin, ipilimumab, iratumumab, labetuzumab (CEA-CIDE®), lexatumumab, lintuzumab, lucatumumab, lumiliximab, mapatumumab, matuzumab, milatuzumab, minretumomab, mitumomab, nacolomab tafenatox, naptumomab estafenatox, necitumumab, nimotuzumab (THERACIM®, THERALOC®), nofetumomab merpentan (VERLUMA®), ofatumumab (ARZERRA®), olaratumab, oportuzumab monatox, oregovomab (OVAREX®), panitumumab (VECTIBIX®), pemtumomab (THERAGYN®), pertuzumab (OMNITARG®), pintumomab, pritumumab, ramucirumab, ranibizumab (LUCENTIS®), rilotumumab, rituximab (MABTHERA®, RITUXAN®), robatumumab, satumomab pendetide, sibrotuzumab, siltuximab, sontuzumab, tacatuzumab tetraxetan (AFP-CIDE®), taplitumomab paptox, tenatumomab, TGN1412, ticilimumab (tremelimumab), tigatuzumab, TNX-650, tositumomab (BEXXAR®), trastuzumab (HERCEPTIN®), tremelimumab, tucotuzumab celmoleukin, veltuzumab, volociximab, votumumab (HUMASPECT®), zalutumumab (HUMAX-EGFR®), and zanolimumab (HUMAX-CD4@).

In other embodiments, the multispecific or multifunctional molecule is administered in combination with a viral cancer therapeutic agent. Exemplary viral cancer therapeutic agents include, but not limited to, vaccinia virus (vvDD-CDSR), carcinoembryonic antigen-expressing measles virus, recombinant vaccinia virus (TK-deletion plus GM-CSF), Seneca Valley virus-001, Newcastle virus, coxsackie virus A21, GL-ONC1, EBNA1 C-terminal/LMP2 chimeric protein-expressing recombinant modified vaccinia Ankara vaccine, carcinoembryonic antigen-expressing measles virus, G207 oncolytic virus, modified vaccinia virus Ankara vaccine expressing p53, OncoVEX GM-CSF modified herpes-simplex 1 virus, fowlpox virus vaccine vector, recombinant vaccinia prostate-specific antigen vaccine, human papillomavirus 16/18 L1 virus-like particle/AS04 vaccine, MVA-EBNA1/LMP2 Inj. vaccine, quadrivalent HPV vaccine, quadrivalent human papillomavirus (types 6, 11, 16, 18) recombinant vaccine (GARDASIL®), recombinant fowlpox-CEA(6D)/TRICOM vaccine; recombinant vaccinia-CEA(6D)-TRICOM vaccine, recombinant modified vaccinia Ankara-5T4 vaccine, recombinant fowlpox-TRICOM vaccine, oncolytic herpes virus NV1020, HPV L1 VLP vaccine V504, human papillomavirus bivalent (types 16 and 18) vaccine (CERVARIX®), herpes simplex virus HF10, Ad5CMV-p53 gene, recombinant vaccinia DF3/MUC1 vaccine, recombinant vaccinia-MUC-1 vaccine, recombinant vaccinia-TRICOM vaccine, ALVAC MART-1 vaccine, replication-defective herpes simplex virus type I (HSV-1) vector expressing human Preproenkephalin (NP2), wild-type reovirus, reovirus type 3 Dearing (REOLYSIN®), oncolytic virus HSV1716, recombinant modified vaccinia Ankara (MVA)-based vaccine encoding Epstein-Barr virus target antigens, recombinant fowlpox-prostate specific antigen vaccine, recombinant vaccinia prostate-specific antigen vaccine, recombinant vaccinia-B7.1 vaccine, rAd-p53 gene, Ad5-delta24RGD, HPV vaccine 580299, JX-594 (thymidine kinase-deleted vaccinia virus plus GM-CSF), HPV-16/18 L1/AS04, fowlpox virus vaccine vector, vaccinia-tyrosinase vaccine, MEDI-517 HPV-16/18 VLP AS04 vaccine, adenoviral vector containing the thymidine kinase of herpes simplex virus TK99UN, HspE7, FP253/Fludarabine, ALVAC(2) melanoma multi-antigen therapeutic vaccine, ALVAC-hB7.1, canarypox-hIL-12 melanoma vaccine, Ad-REIC/Dkk-3, rAd-IFN SCH 721015, TIL-Ad-INFg, Ad-ISF35, and coxsackievirus A21 (CVA21, CAVATAK®).

In other embodiments, the multispecific or multifunctional molecule is administered in combination with a nanopharmaceutical. Exemplary cancer nanopharmaceuticals include, but not limited to, ABRAXANE® (paclitaxel bound albumin nanoparticles), CRLX101 (CPT conjugated to a linear cyclodextrin-based polymer), CRLX288 (conjugating docetaxel to the biodegradable polymer poly (lactic-co-glycolic acid)), cytarabine liposomal (liposomal Ara-C, DEPOCYT™), daunorubicin liposomal (DAUNOXOME®), doxorubicin liposomal (DOXIL®, CAELYX®), encapsulated-daunorubicin citrate liposome (DAUNOXOME®), and PEG anti-VEGF aptamer (MACUGEN®).

In some embodiments, the multispecific or multifunctional molecule is administered in combination with paclitaxel or a paclitaxel formulation, e.g., TAXOL®, protein-bound paclitaxel (e.g., ABRAXANE®). Exemplary paclitaxel formulations include, but are not limited to, nanoparticle albumin-bound paclitaxel (ABRAXANE®, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Bioorganic & Medicinal Chemistry Letters (2007) 17:617-620).

Exemplary RNAi and antisense RNA agents for treating cancer include, but not limited to, CALAA-01, siG12D LODER (Local Drug EluteR), and ALN-VSP02.

Other cancer therapeutic agents include, but not limited to, cytokines (e.g., aldesleukin (IL-2, Interleukin-2, PROLEUKIN®), alpha Interferon (IFN-alpha, Interferon alfa, INTRON® A (Interferon alfa-2b), ROFERON-A® (Interferon alfa-2a)), Epoetin alfa (PROCRIT®), filgrastim (G-CSF, Granulocyte—Colony Stimulating Factor, NEUPOGEN®), GM-CSF (Granulocyte Macrophage Colony Stimulating Factor, sargramostim, LEUKINE™), IL-11 (Interleukin-11, oprelvekin, NEUMEGA®), Interferon alfa-2b (PEG conjugate) (PEG interferon, PEG-INTRON™), and pegfilgrastim (NEULASTA™)), hormone therapy agents (e.g., aminoglutethimide (CYTADREN®), anastrozole (ARIMIDEX®), bicalutamide (CASODEX®), exemestane (AROMASIN®), fluoxymesterone (HALOTESTIN®), flutamide (EULEXIN®), fulvestrant (FASLODEX®), goserelin (ZOLADEX®), letrozole (FEMARA®), leuprolide (ELIGARD™, LUPRON®, LUPRON DEPOT®, VIADUR™), megestrol (megestrol acetate, MEGACE®), nilutamide (ANANDRON®, NILANDRON®), octreotide (octreotide acetate, SANDOSTATIN®, SANDOSTATIN LAR®), raloxifene (EVISTA®), romiplostim (NPLATE®), tamoxifen (NOVALDEX®), and toremifene (FARESTON®)), phospholipase A2 inhibitors (e.g., anagrelide (AGRYLIN®)), biologic response modifiers (e.g., BCG (THERACYS®, TICE®), and Darbepoetin alfa (ARANESP®)), target therapy agents (e.g., bortezomib (VELCADE®), dasatinib (SPRYCEL™), denileukin diftitox (ONTAK®), erlotinib (TARCEVA®), everolimus (AFINITOR®), gefitinib (IRESSA®), imatinib mesylate (STI-571, GLEEVEC™), lapatinib (TYKERB®), sorafenib (NEXAVAR®), and SU11248 (sunitinib, SUTENT®)), immunomodulatory and antiangiogenic agents (e.g., CC-5013 (lenalidomide, REVLIMID®), and thalidomide (THALOMID®)), glucocorticosteroids (e.g., cortisone (hydrocortisone, hydrocortisone sodium phosphate, hydrocortisone sodium succinate, ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE®, MEDRALONE®, MEDROL®, M-PREDNISOL®, SOLU-MEDROL®), prednisolone (DELTA-CORTEF®, ORAPRED®, PEDIAPRED®, PRELONE®), and prednisone (DELTASONE®, LIQUID PRED®, METICORTEN®, ORASONE®)), and bisphosphonates (e.g., pamidronate (AREDIA®), and zoledronic acid (ZOMETA®)) In some embodiments, the multispecific or multifunctional molecule is used in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., an antibody against VEGF, a VEGF trap, a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-8 inhibitor)), a RAF-1 inhibitor, a KIT inhibitor and a RET inhibitor. In some embodiments, the anti-cancer agent used in combination with the AHCM agent is selected from the group consisting of: axitinib (AG013736), bosutinib (SKI-606), cediranib (RECENTIN™ AZD2171), dasatinib (SPRYCEL®, BMS-354825), erlotinib (TARCEVA®), gefitinib (TRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951 (tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, Pazopanib Hydrochloride, PD173074, nSorafenib Tosylate (Bay 43-9006), SU 5402, TSU-68 (SU6668), vatalanib, XL880 (GSK1363089, EXEL-2880). Selected tyrosine kinase inhibitors are chosen from sunitinib, erlotinib, gefitinib, or sorafenib. In one embodiment, the tyrosine kinase inhibitor is sunitinib.

In one embodiment, the multispecific or multifunctional molecule is administered in combination with one of more of: an anti-angiogenic agent, or a vascular targeting agent or a vascular disrupting agent. Exemplary anti-angiogenic agents include, but are not limited to, VEGF inhibitors (e.g., anti-VEGF antibodies (e.g., bevacizumab); VEGF receptor inhibitors (e.g., itraconazole); inhibitors of cell proliferatin and/or migration of endothelial cells (e.g., carboxyamidotriazole, TNP-470); inhibitors of angiogenesis stimulators (e.g., suramin), among others. A vascular-targeting agent (VTA) or vascular disrupting agent (VDA) is designed to damage the vasculature (blood vessels) of cancer tumors causing central necrosis (reviewed in, e.g., Thorpe, P. E. (2004) Clin. Cancer Res. Vol. 10:415-427). VTAs can be small-molecule. Exemplary small-molecule VTAs include, but are not limited to, microtubule destabilizing drugs (e.g., combretastatin A-4 disodium phosphate (CA4P), ZD6126, AVE8062, Oxi 4503); and vadimezan (ASA404).

### Immune Checkpoint Inhibitors

In other embodiments, methods described herein comprise use of an immune checkpoint inhibitor in combination with the multispecific or multifunctional molecule. The methods can be used in a therapeutic protocol in vivo.

In embodiments, an immune checkpoint inhibitor inhibits a checkpoint molecule. Exemplary checkpoint molecules include but are not limited to CTLA4, PD1, PD-L1, PD-L2, TIM3, LAG3, CD160, 2B4, CD80, CD86, B7-H3 (CD276), B7-H4 (VTCN1), HVEM (TNFRSF14 or CD270), BTLA, KIR, MHC class I, MHC class II, GAL9, VISTA, BTLA, TIGIT, LAIR1, and A2aR. See, e.g., Pardoll. Nat. Rev. Cancer 12.4(2012):252-64, incorporated herein by reference.

In embodiments, the immune checkpoint inhibitor is a PD-1 inhibitor, e.g., an anti-PD-1 antibody such as Nivolumab, Pembrolizumab or Pidilizumab. Nivolumab (also called MDX-1106, MDX-1106-04, ONO-4538, or BMS-936558) is a fully human IgG4 monoclonal antibody that specifically inhibits PD1. See, e.g., U.S. Pat. No. 8,008,449 and WO2006/121168. Pembrolizumab (also called Lambrolizumab, MK-3475, MK03475, SCH-900475 or KEYTRUDA®; Merck) is a humanized IgG4 monoclonal antibody that binds to PD-1. See, e.g., Hamid, O. et al. (2013) New England Journal of Medicine 369 (2): 134-44, U.S. Pat. No. 8,354,509 and WO2009/114335. Pidilizumab (also called CT-011 or Cure Tech) is a humanized IgG1k monoclonal antibody that binds to PD1. See, e.g., WO2009/101611. In one embodiment, the inhibitor of PD-1 is an antibody molecule having a sequence substantially identical or similar thereto, e.g., a sequence at least 85%, 90%, 95% identical or higher to the sequence of Nivolumab, Pembrolizumab or Pidilizumab. Additional anti-PD1 antibodies, e.g., AMP 514 (Amplimmune), are described, e.g., in U.S. Pat. No. 8,609,089, US 2010028330, and/or US 20120114649.

In some embodiments, the PD-1 inhibitor is an immunoadhesin, e.g., an immunoadhesin comprising an extracellular/PD-1 binding portion of a PD-1 ligand (e.g., PD-L1 or PD-L2) that is fused to a constant region (e.g., an Fc region of an immunoglobulin). In embodiments, the PD-1 inhibitor is AMP-224 (B7-DCIg, e.g., described in WO2011/066342 and WO2010/027827), a PD-L2 Fc fusion soluble receptor that blocks the interaction between B7-H1 and PD-1.

In embodiments, the immune checkpoint inhibitor is a PD-L1 inhibitor, e.g., an antibody molecule. In some embodiments, the PD-L1 inhibitor is YW243.55.S70, MPDL3280A, MEDI-4736, MSB-0010718C, or MDX-1105. In some embodiments, the anti-PD-L1 antibody is MSB0010718C (also called A09-246-2; Merck Serono), which is a monoclonal antibody that binds to PD-L1. Exemplary humanized anti-PD-L1 antibodies are described, e.g., in WO2013/079174. In one embodiment, the PD-L1 inhibitor is an anti-PD-L1 antibody, e.g., YW243.55.S70. The YW243.55.S70 antibody is described, e.g., in WO 2010/077634. In one embodiment, the PD-L1 inhibitor is MDX-1105 (also called BMS-936559), which is described, e.g., in WO2007/005874. In one embodiment, the PD-L1 inhibitor is MDPL3280A (Genentech/Roche), which is a human Fc-optimized IgG1 monoclonal antibody against PD-L1. See, e.g., U.S. Pat. No. 7,943,743 and U.S Publication No.: 20120039906. In one embodiment, the inhibitor of PD-L1 is an antibody molecule having a sequence substantially identical or similar thereto, e.g., a sequence at least 85%, 90%, 95% identical or higher to the sequence of YW243.55.S70, MPDL3280A, MEDI-4736, MSB-0010718C, or MDX-1105.

In embodiments, the immune checkpoint inhibitor is a PD-L2 inhibitor, e.g., AMP-224 (which is a PD-L2 Fc fusion soluble receptor that blocks the interaction between PD1 and B7-H1. See, e.g., WO2010/027827 and WO2011/066342.

In one embodiment, the immune checkpoint inhibitor is a LAG-3 inhibitor, e.g., an anti LAG-3 antibody molecule. In embodiments, the anti-LAG-3 antibody is BMS-986016 (also called BMS986016; Bristol-Myers Squibb). BMS-986016 and other humanized anti-LAG-3 antibodies are described, e.g., in US 2011/0150892, WO2010/019570, and WO2014/008218.

In embodiments, the immune checkpoint inhibitor is a TIM-3 inhibitor, e.g., anti-TIM3 antibody molecule, e.g., described in U.S. Pat. No. 8,552,156, WO 2011/155607, EP 2581113 and U.S Publication No.: 2014/044728.

In embodiments, the immune checkpoint inhibitor is a CTLA-4 inhibitor, e.g., anti-CTLA-4 antibody molecule. Exemplary anti-CTLA4 antibodies include Tremelimumab (IgG2 monoclonal antibody from Pfizer, formerly known as ticilimumab, CP-675,206); and Ipilimumab (also called MDX-010, CAS No. 477202-00-9). Other exemplary anti-CTLA-4 antibodies are described, e.g., in U.S. Pat. No. 5,811,097.

### INCORPORATION BY REFERENCE

All publications and patents mentioned herein are hereby incorporated by reference in their entirety as if each individual publication or patent was specifically and individually indicated to be incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1.-138. (canceled)

139. A multispecific molecule, comprising:
(a) a first tumor-targeting moiety that binds a to G6B; and
(b) a second tumor-targeting moiety that binds to CD34.

140. The multispecific molecule of claim 139, wherein the first tumor-targeting moiety comprises a heavy chain variable region (VH) comprising a heavy chain complementarity determining region 1 (VHCDR1), a heavy chain complementarity determining region 2 (VHCDR2), and a heavy chain complementarity determining region 3 (VHCDR3) of SEQ ID NO: 87, SEQ ID NO: 88, and SEQ ID NO: 89, respectively; and a light chain variable region (VL) comprising a light chain complementarity determining region 1 (VLCDR1), a light chain complementarity determining region 2 (VLCDR2), and a light chain complementarity determining region 3 (VLCDR3) of SEQ ID NO: 90, SEQ ID NO: 91, and SEQ ID NO: 92, respectively.

141. The multifunctional molecule of claim 139, wherein the first tumor-targeting moiety comprises a VH comprising an amino acid sequence with at least 80% sequence identity to SEQ ID NO: 1.

142. The multispecific molecule of claim 139, wherein the first tumor-targeting moiety comprises a VL comprising an amino acid sequence with at least 80% sequence identity to SEQ ID NO: 2.

143. The multispecific molecule of claim 139, wherein the first tumor-targeting moiety comprises a VH comprising an amino acid sequence with at least 80% sequence identity to SEQ ID NO: 1 and a VL comprising an amino acid sequence with at least 80% sequence identity to SEQ ID NO: 2.

144. The multispecific molecule of claim 139, wherein the first tumor-targeting moiety comprises a VH comprising the amino acid sequence of SEQ ID NO: 1 and a VL comprising the amino acid sequence of SEQ ID NO: 2.

145. The multispecific molecule of claim 139, wherein the multispecific molecule comprises a full-length antibody, a Fab, a F(ab′)2, an Fv, a single chain Fv (scFv), a half arm antibody, a diabody, a bivalent antibody, a monovalent antibody, a bispecific antibody, or a camelid antibody.

146. The multispecific molecule of claim 139, wherein the multispecific molecule comprises at least two non-contiguous polypeptide chains comprising a first polypeptide and a second polypeptide chain,
wherein the first polypeptide comprises a first member of a dimerization module and the second polypeptide comprises a second member of the dimerization module,
wherein the first polypeptide and the second polypeptide form a complex via the first member of the dimerization module and the second member of the dimerization module, and
wherein the first member of the dimerization module comprises a first heavy chain constant region and the second member of the dimerization module comprises a second heavy chain constant region.

147. The multispecific molecule of claim 146, wherein the heavy chain constant region is selected from the group consisting of IgG1 or fragment thereof, IgG2 or fragment thereof, IgG3 or fragment thereof, and IgG4 or fragment thereof.

148. The multispecific molecule of claim 146, wherein the first heavy chain constant region comprises a first Fc region, and the second heavy chain constant region comprises a second Fc region.

149. The multispecific molecule of claim 148, wherein the first Fc region or the second Fc region comprise an amino acid substitution at a position selected from one or more of 347, 349, 350, 351, 366, 368, 370, 392, 394, 395, 397, 398, 399, 405, 407, and 409 of the Fc region of human IgG1.

150. The multispecific molecule of claim 148, wherein first Fc region comprises an amino acid substitution selected from the group consisting of T366S, L368A, and Y407V and the second Fc region comprises an amino acid substitution of T366W, or the first Fc region comprises an amino acid substitution of T366W and the second Fc region comprises an amino acid substitution selected from the group consisting of T366S, L368A, and Y407V.

151. The multispecific molecule of claim 139, wherein the first tumor-targeting moiety, the second tumor-targeting moiety, or a combination thereof comprises a light chain constant region selected from the group consisting of a human kappa light chain constant region and a human lambda light chain constant region.

152. The multispecific molecule of claim 139, wherein the multispecific molecule further comprises an immune cell engager, a cytokine molecule, a modulator of a cytokine molecule, a stromal modifying moiety, or any combination thereof, and
wherein the immune cell engager is selected from the group consisting of a T cell engager, an NK cell engager, a B cell engager, a dendritic cell engager, and a macrophage cell engager.

153. A pharmaceutical composition comprising the composition of claim 139, and a pharmaceutically acceptable carrier, excipient, or stabilizer.

154. A method of treating cancer in a subject in need thereof comprising administering to the subject a therapeutically effective amount of the composition of claim 139, wherein the administering is effective to treat the cancer in the subject.

155. The method of claim 154, wherein the cancer is a solid tumor cancer or a hematological cancer.

156. The method of claim 155, wherein the cancer is the hematological cancer and wherein the cancer is a myeloproliferative neoplasm.

157. The method of claim 156, wherein the myeloproliferative neoplasm is selected from a group consisting of primary or idiopathic myelofibrosis (MF), essential thrombocytosis (ET), polycythemia vera (PV), or chronic myelogenous leukemia (CML).

158. The method of claim 156, wherein the cancer is myelofibrosis.
